(19) United States
(12) Patent Application Publication
(10) Pub. No.: US 2025/0135028 A1
(43) Pub. Date: May 1, 2025

(54) TUNABLE POLYMERIC NANOPARTICLES FOR TARGETED AND SUSTAINED DELIVERY OF GLYCOPEPTIDE ANTIBIOTICS

(71) Applicant: Insmed Incorporated, Bridgewater, NJ (US)

(72) Inventors: Xizhe ZHAO, Bridgewater, NJ (US); Adam J. PLAUNT, Bridgewater, NJ (US); Vladimir MALININ, Bridgewater, NJ (US); Yang DENG, Green Brook, NJ (US); Kuan-Ju CHEN, Bridgewater, NJ (US); Donna M. KONICEK, Bridgewater, NJ (US); Walter R. PERKINS, Bridgewater, NJ (US)

(21) Appl. No.: 18/685,116

(22) PCT Filed: Aug. 19, 2022
(86) PCT No.: PCT/US2022/040864

§ 371 Date: Feb. 20, 2024

Related U.S. Application Data
(60) Provisional application No. 63/235,467, filed on Aug. 20, 2021.

(51) Int. Cl. A61K 47/69; A61K 9/51; B82Y 5/00; C07K 16/12; C08G 69/40

(52) U.S. Cl. CPC A61K 47/6937; A61K 9/5153; C07K 16/12; C08G 69/40; B82Y 5/00

(57) ABSTRACT

The present disclosure provides copolymers and glycopeptide antibiotic-loaded polymeric nanoparticles (PNPs) comprising the copolymers as well as charge neutral polymers for targeted and sustained delivery of glycopeptide antibiotics to treat bacterial infections, including but not limited to biofilm bacterial infections. The copolymers are block, alternate, or random copolymers comprising x units of the formula

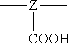

and y units of the formula

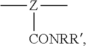

wherein Z is an organic moiety, R and R′ are each independently selected from the group consisting of hydrogen and C₁-C₁₈ alkyl, each of x and y is an integer of 1 or greater, the sum of x and y is an integer from about 40 to about 714, and y is from about 10% to about 90% of the sum of x and y.

### CROSS REFERENCE TO RELATED APPLICATION

This application is a National Stage of International Patent Application Number PCT/US2022/040864, filed Aug. 19, 2022, which claims priority from U.S. Provisional Application No. 63/235,467, filed Aug. 20, 2021, the disclosure of each of which is incorporated by reference herein in its entirety.

### BACKGROUND OF THE INVENTION

Glycopeptide antibiotics, such as vancomycin and its derivatives, have been used historically to treat certain bacterial infections, such as infections caused by Gram-positive bacteria, e.g., methicillin-resistant Staphylococcus aureus (MRSA). However, limitations exist in administering glycopeptide antibiotics to treat bacterial infections that require a sustained and high local concentration of the antibiotics, e.g., a biofilm bacterial infection, with frequent administration of high doses of glycopeptide antibiotics leading to systemic toxicities.

The present invention addresses the need in the art for providing high local concentrations of glycopeptide antibiotics while minimizing systemic exposure to the glycopeptide antibiotics.

### SUMMARY OF THE INVENTION

The present application provides copolymers and glycopeptide antibiotic-loaded polymeric nanoparticles (PNPs) comprising the copolymers as well as charge neutral polymers for targeted and sustained delivery of glycopeptide antibiotics to treat bacterial infections, including but not limited to biofilm bacterial infections. With a tunable negative charge density-to-hydrophobicity ratio, the copolymers of the PNPs can be customized to efficiently load and encapsulate positively charged glycopeptide antibiotics and stabilize the PNPs. Additionally, a targeting moiety can be attached to the PNPs for enhanced targeted delivery of glycopeptide antibiotics to the sites of infections.

In one aspect, the present disclosure relates to a block, alternate, or random copolymer comprising x units of the formula

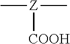

and y units of the formula

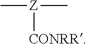

Z is an organic moiety. R and R′ are each independently selected from the group consisting of hydrogen and C₁-C₁₈ alkyl. Each of x and y is an integer of 1 or greater. The sum of x and y is an integer from about 40 to about 714, and y is from about 10% to about 90% of the sum of x and y. In a further embodiment,

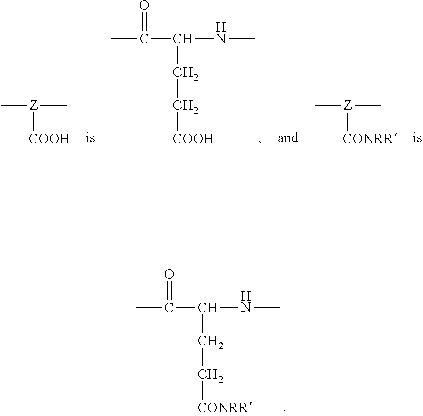

In one embodiment, the copolymer is a random copolymer. In one embodiment, the copolymer is of Formula (I):

P—R² (I),

wherein R² is hydrogen or C₁-C₆ alkyl, P is a random copolymer moiety comprising x units of the formula

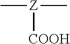

and y units of the formula

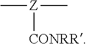

In a further embodiment, the copolymer of Formula (I) does not include a polyethylene glycol (PEG) moiety. In a further embodiment, P is a random copolymer moiety comprising x units of the formula

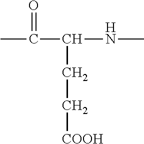

and y units of the formula

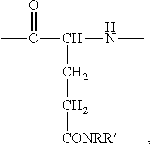

and one of R and R′ is linear C₁₀ alkyl and the other hydrogen. In a further embodiment, R² is hydrogen. In a further embodiment, the sum of x and y is about 155, and y is about 30% of the sum of x and y.

In another embodiment, the copolymer is of Formula (II):

PEG-L-P′ (II),

wherein, L is a bond or a linker covalently connecting PEG and P′, PEG is a PEG moiety with a molecular weight of from about 500 g/mol to about 20000 g/mol, and P′ comprises a block, alternate, or random copolymer comprising x units of the formula

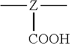

and y units of the formula

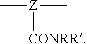

In a further embodiment, P′ comprises a random copolymer comprising x units of the formula

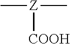

and y units of the formula

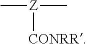

In a further embodiment, L is —CH₂—CH₂—NH—, P′ is P″—R², and the copolymer is of Formula (IIa):

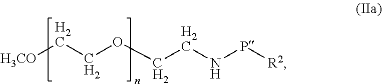

wherein n is an integer from about 22 to about 227, R² is hydrogen or C₁-C₆ alkyl, P″
is a random copolymer moiety comprising x units of the formula

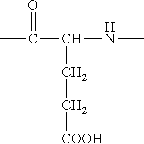

and y units of the formula

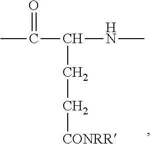

one of R and R′ is linear C₁₀ alkyl and the other hydrogen, and the ratio of the sum of x and y to n is from about 0.7 to about 7. In a further embodiment, R² is hydrogen. In a further embodiment, n is about 45, the sum of x and y is about 155, and y is about 36% of the sum of x and y.

In another aspect, the present disclosure relates to a pharmaceutical composition comprising nanoparticles comprising a glycopeptide antibiotic complexed with one or more copolymers disclosed herein and a charge neutral polymer selected from the group consisting of polylactic acid, polylactic acid-polyethylene glycol (PLA-PEG), poly(lactic-co-glycolic acid), a polyester-polyethylene glycol (polyester-PEG), and a combination thereof. The glycopeptide antibiotic is positively charged at a pH of from about 6 to about 8.

In one embodiment of the pharmaceutical composition, the one or more copolymers does not include a PEG moiety, and the charge neutral polymer is PLA-PEG. In a further embodiment, the one or more copolymers which does not include a PEG moiety is a copolymer of Formula (I).

In another embodiment of the pharmaceutical composition, the one or more copolymers is a copolymer of Formula (II) or (IIa), and the charge neutral polymer is PLA-PEG.

In one embodiment of the pharmaceutical composition, the glycopeptide antibiotic is RV40, RV94, or oritavancin.

In one embodiment of the pharmaceutical composition, a targeting moiety is attached to the nanoparticles for targeting the nanoparticles to a bacterial biofilm or the surface of a bacteria. In a further embodiment, the targeting moiety is attached to the charge neutral polymer of the nanoparticles. In a further embodiment, the targeting moiety is attached to the PEG moiety of PLA-PEG. In a further embodiment, the targeting moiety is an anti-polysaccharide intercellular adhesin (PIA) antibody. In a further embodiment, the anti-PIA antibody is attached to the PEG moiety of PLA-PEG via interaction between biotin and avidin, or between biotin and streptavidin.

In another aspect, the present disclosure relates to a method for treating a bacterial infection in a patient in need thereof. The method includes administering an effective amount of one of the pharmaceutical compositions disclosed herein to the patient in need of treatment.

In one embodiment of the method, the bacterial infection is a Gram-positive bacterial infection. In a further embodiment, the Gram-positive bacterial infection is a Staphylococcus aureus (S. aureus) infection. In a further embodiment, the S. aureus infection is a methicillin-resistant S. aureus (MRSA) infection. In a further embodiment, the MRSA infection is a MRSA biofilm infection.

In one embodiment of the method, the patient is a cystic fibrosis patient. In another embodiment, the patient is an osteomyelitis patient.

### BRIEF DESCRIPTION OF THE FIGURES

FIG. 1 is a schematic showing loading and encapsulation of positively charged antibiotics with polymeric nanoparticles comprising alkyl chain modified poly (L-glutamic acid) (pGlu) and polylactic acid (PLA)-PEG according to Example 1.

FIG. 2 shows the reaction scheme for synthesizing pGlu(0.6C₁₀) according to Example 1.

FIG. 3 shows the ¹H NMR data and proton assignment of pGlu (0.6C₁₀) according to Example 1.

FIG. 4 is a schematic showing fabrication of various drug-loaded polymeric nanoparticle (PNP) formulations according to Example 1.

FIG. 5 is a graph showing the fluorescence intensity in the supernatant at various time points following incubation of RV40-PNP in PBS or plasma according to Example 2.

FIG. 6 is a graph showing the percentage of RV40 released into the supernatant at various time points following incubation of RV40-PNP in PBS or plasma according to Example 2.

FIG. 7 is a schematic showing the modification of poly(L-glutamic acid)-block-poly(ethylene glycol) (pGlu-PEG) with alkylamine, and the loading and encapsulation of positively charged antibiotics with polymeric nanoparticles comprising the alkyl-modified pGlu-PEG according to Example 3.

FIG. 8 shows the reaction scheme for synthesizing pGlu(0.6C₁₀)-PEG according to Example 3.

FIG. 9 shows the ¹H NMR data and proton assignment of pGlu(0.6C₁₀)-PEG according to Example 3.

FIG. 10 is a schematic showing fabrication of various drug-loaded polymeric nanoparticle (PNP) formulations according to Example 3.

FIG. 11 is a graph showing plasma PK data following intravenous injection of 6 mg/kg of ORI-hybrid PNP, ORI-PLA PNP, or ORI to osteomyelitis rats according to Example 4.

FIG. 12A is a graph showing the mean concentrations of oritavancin in the uninfected and infected tibias of osteomyelitis rats treated with ORI-hybrid PNP, ORI-PLA PNP, or ORI according to Example 4.

FIG. 12B is a graph showing the ratios of the mean oritavancin concentration in the infected tabia to the mean oritavancin concentration in the uninfected tabia of osteomyelitis rats treated with ORI-hybrid PNP, ORI-PLA PNP, or ORI according to Example 4.

FIG. 13 is a graph showing the ratios of the fluorescence intensity in the infected tibia to that in the uninfected tibia via in vivo whole-body imaging of the osteomyelitis rat model, following administration of the fluorescence-labeled RV40-PNP formulations as indicated according to Example 5.

### DETAILED DESCRIPTION OF THE INVENTION

Throughout the present disclosure, the term “about” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or there below.

“Pharmaceutically acceptable salt” includes both acid and base addition salts. A pharmaceutically acceptable acid addition salt refers to those salts which retain the biological effectiveness and properties of the free bases, which are not biologically or otherwise undesirable, and which are formed with inorganic acids such as, but are not limited to, hydrochloric acid (HCl), hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, acetic acid (e.g., as acetate), tartaric acid, thiocyanic acid, p-toluenesulfonic acid, trifluoroacetic acid (TFA), undecylenic acid, and the like. In one embodiment, the pharmaceutically acceptable salt is HCl, TFA, lactate or acetate. In a further embodiment, the pharmaceutically acceptable salt is a lactic salt.

A pharmaceutically acceptable base addition salt retains the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Inorganic salts include the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Organic bases that can be used to form a pharmaceutically acceptable salt include isopropylamine, diethylamine, ethanolamine, trimethylamine, dicyclohexylamine, choline and caffeine.

Throughout the present disclosure, numerical ranges are provided for certain quantities. It is to be understood that these ranges comprise all subranges therein. Thus, the range “from 50 to 80” includes all possible ranges therein (e.g., from 51 to 79, from 52 to 78, from 53 to 77, from 54 to 76, from 55 to 75, from 60 to 70, etc.). Furthermore, all values within a given range may be an endpoint for the range encompassed thereby (e.g., the range of from 50 to 80 includes the ranges with endpoints such as from 55 to 80, from 50 to 75, etc.).

The term “treating” in one embodiment, includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (e.g., causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In another embodiment, “treating” refers to relieving the condition (for example, by causing regression of the state, disorder or condition or at least one of its clinical or subclinical symptoms). The benefit to a subject to be treated is either statistically significant as compared to the state or condition of the same subject before the treatment, or as compared to the state or condition of an untreated control subject, or the benefit is at least perceptible to the subject or to the physician.

“Effective amount” means an amount of a pharmaceutical composition or the active pharmaceutical ingredient (API) in the pharmaceutical composition, i.e., a glycopeptide antibiotic, of the present disclosure that is sufficient to result in the desired therapeutic response.

In the present disclosure, when the compounds and formulae are set forth graphically without depicting stereochemistry, one of ordinary skill in the art will understand that the compounds described herein each have a stereochemical configuration. In some embodiments, a stereoisomer (e.g., enantiomer, diastereomer) or a combination of stereoisomers of the respective compounds are provided.

In one aspect, the present disclosure relates to a block, alternate, or random copolymer comprising x units of the formula

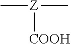

and y units of the formula

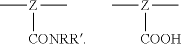

represents a unit of carboxylic acid, and

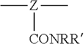

represents a unit of corresponding carboxamide. Z represents an organic moiety. R and R′ are each independently selected from the group consisting of hydrogen and C₁-C₁₈ alkyl. Each of x and y is an integer of 1 or greater. The sum of x and y is an integer from about 40 to about 714, with y being from about 10% to about 90% of the sum of x and y.

In one embodiment, the copolymer is a random copolymer.

In one embodiment, the copolymer does not include a polyethylene glycol (PEG) moiety.

In one embodiment, the copolymer is of Formula (I):

P-R² (I),

wherein R² is hydrogen or C₁-C₆ alkyl, P is a random copolymer moiety comprising x. units of the formula

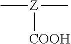

and y units of the formula

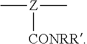

In a further embodiment, the copolymer of Formula (I) does not include a PEG moiety.

In one embodiment of the copolymer of Formula (I), R² is hydrogen. In another embodiment, R² is methyl. In another embodiment, R² is ethyl. In another embodiment, R² is linear C₃-C₆ alkyl.

In another embodiment, the copolymer is of Formula (II) which includes a PEG moiety:

PEG-L-P′ (II),

wherein L is a bond or a linker covalently connecting PEG and P′, PEG is a PEG moiety with a molecular weight of from about 500 g/mol to about 20,000 g/mol, and P′ comprises a block, alternate, or random copolymer comprising x units of the formula

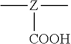

and y units of the formula

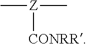

In a further embodiment, P′ comprises a random copolymer comprising x units of the formula

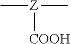

and y units of the formula

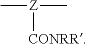

In one embodiment of the copolymer of Formula (II), the PEG moiety has a molecular weight of from about 1,000 g/mol to about 15,000 g/mol. In another embodiment, the PEG moiety has a molecular weight of from about 1,000 g/mol to about 10,000 g/mol. In another embodiment, the PEG moiety has a molecular weight of from about 1,000 g/mol to about 7,500 g/mol. In another embodiment, the PEG moiety has a molecular weight of from about 1,000 g/mol to about 5,000 g/mol. In another embodiment, the PEG moiety has a molecular weight of from about 1,000 g/mol to about 3,000 g/mol. In a further embodiment, the PEG moiety has a molecular weight of about 2,000 g/mol.

In one embodiment of the copolymer of Formula (II), L is a linker represented by —(CH₂)ₙ—NH—, and n is an integer from 1 to 6. In a further embodiment, n is 2, i.e., L is —CH₂—CH₂—NH—.

In one embodiment of the copolymers disclosed herein, R and R′ are each independently selected from the group consisting of hydrogen and C₃-C₁₅ alkyl. In another embodiment, R and R′ are each independently selected from the group consisting of hydrogen and C₃-C₁₃ alkyl. In another embodiment, R and R′ are each independently selected from the group consisting of hydrogen and C₅-C₁₂ alkyl. In another embodiment, R and R′ are each independently selected from the group consisting of hydrogen and C₇-C₁₁ alkyl. In another embodiment, R and R′ are each independently selected from the group consisting of hydrogen and C₁₀ alkyl.

In one embodiment of the copolymers, one of R and R′ is linear C₁-C₁₈ alkyl and the other hydrogen. In a further embodiment, one of R and R′ is linear C₃-C₁₆ alkyl and the other hydrogen. In a further embodiment, one of R and R′ is linear C₅-C₁₄ alkyl and the other hydrogen. In a further embodiment, one of R and R′ is linear C₆-C₁₃ alkyl and the other hydrogen, i.e., one of R and R′ is linear C₆ alkyl, linear C₇ alkyl, linear C₈ alkyl, linear C₉ alkyl, linear C₁₀ alkyl, linear C₁₁ alkyl, linear C₁₂ alkyl, or linear C₁₃ alkyl, and the other hydrogen. In a further embodiment, one of R and R′ is linear C₁₀ alkyl and the other hydrogen.

In one embodiment of the copolymers, Z is linear C₁-C₆ alkylene, i.e.,

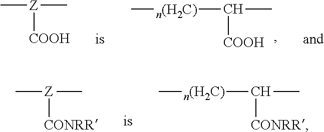

wherein n is 0, 1, 2, 3, 4, or 5. In a further embodiment, n is 1, i.e.,

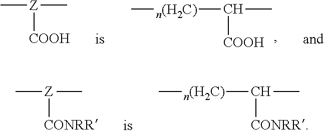

In one embodiment of the copolymers, Z is branched C₁-C₆ alkylene.

In another embodiment of the copolymers,

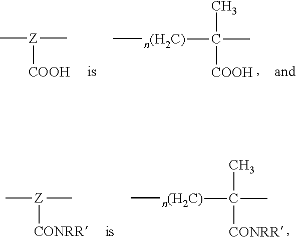

wherein n is 0, 1, 2, 3, 4, or 5. In a further embodiment, n is 1, i.e.,

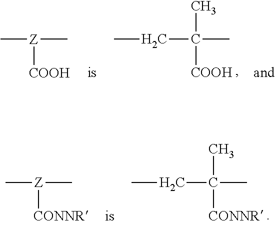

In some embodiments of the copolymers,

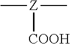

represents a unit of amino acid, and

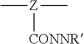

represents a unit of corresponding carboxamide derivative. Accordingly, in one embodiment,

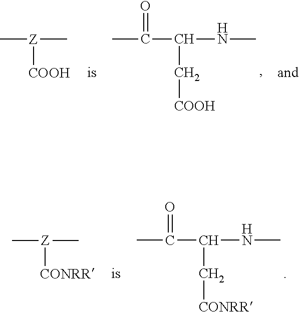

In another embodiment,

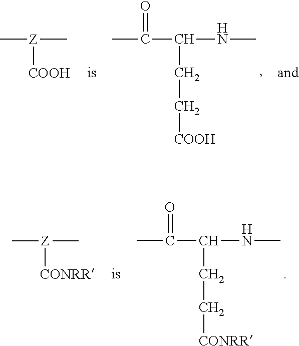

In one embodiment of the copolymers, the sum of x and y is an integer from about 63 to about 675. In another embodiment, the sum of x and y is an integer from about 79 to about 635. In another embodiment, the sum of x and y is an integer from about 119 to about 595. In another embodiment, the sum of x and y is an integer from about 159 to about 556. In another embodiment, the sum of x and y is an integer from about 198 to about 516. In another embodiment, the sum of x and y is an integer from about 238 to about 476. In another embodiment, the sum of x and y is an integer from about 278 to about 437. In another embodiment, the sum of x and y is an integer from about 317 to about 397. In another embodiment, the sum of x and y is an integer from about 79 to about 317. In another embodiment, the sum of x and y is an integer from about 79 to about 238. In another embodiment, the sum of x and y is an integer from about 119 to about 198. In another embodiment, the sum of x and y is an integer from about 150 to about 160.

In one embodiment of the copolymers, y is from about 15% to about 75% of the sum of x and y. In another embodiment, y is from about 20% to about 60% of the sum of x and y. In another embodiment, y is from about 20% to about 50% of the sum of x and y. In another embodiment, y is from about 20% to about 40% of the sum of x and y, e.g., about 20%, about 22%, about 24%, about 26%, about 28%, about 30%, about 32%, about 34%, about 36%, about 38%, or about 40%. In another embodiment, y is from about 20% to about 30% of the sum of x and y. In another embodiment, y is from about 30% to about 40% of the sum of x and y.

In one embodiment, the copolymer is of Formula (I), wherein P is a random copolymer moiety comprising x units of the formula

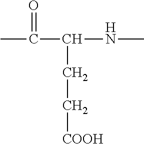

and y units of the formula

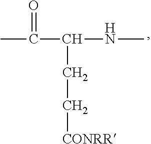

wherein one of R and R′ is linear C₁₀ alkyl and the other hydrogen. In a further embodiment, R² is hydrogen. In a further embodiment, the sum of x and y is about 155, and y is about 30% of the sum of x and y.

In another embodiment, the copolymer is of Formula (II), wherein L is —CH₂—CH₂—NH—, P′ is P″—R², and hence the copolymer is of Formula (IIa):

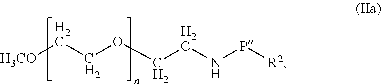

wherein n is an integer from about 22 to about 227, R² is hydrogen or C₁-C₆ alkyl, P″ is a random copolymer moiety comprising x units of the formula

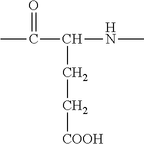

and y units of the formula

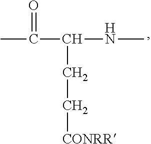

one of R and R′ is linear C₁₀ alkyl and the other hydrogen, and the ratio of the sum of x and y to n is from about 0.7 to about 7.

In one embodiment of the copolymer of Formula (IIa), R² is hydrogen. In another embodiment, R² is methyl. In another embodiment, R² is ethyl. In another embodiment, R² is linear C₃-C₆ alkyl.

In one embodiment of the copolymer of Formula (IIa), n is an integer from about 45 to about 205. In another embodiment, n is an integer from about 68 to about 182. In another embodiment, n is an integer from about 91 to about 159. In another embodiment, n is an integer from about 114 to about 136. In another embodiment, n is an integer from about 23 to about 114. In another embodiment, n is an integer from about 23 to about 91. In another embodiment, n is an integer from about 23 to about 68. In another embodiment, n is about 45.

In one embodiment of the copolymer of Formula (IIa), the ratio of the sum of x and y to n is from about 1 to about 7. In another embodiment, the ratio of the sum of x and y to n is from about 1.2 to about 6.8. In another embodiment, the ratio of the sum of x and y to n is from about 1.5 to about 6.5. In another embodiment, the ratio of the sum of x and y to n is from about 2 to about 6. In another embodiment, the ratio of the sum of x and y to n is from about 2.5 to about 5.5. In another embodiment, the ratio of the sum of x and y to n is from about 3 to about 5. In another embodiment, the ratio of the sum of x and y to n is from about 3 to about 4.

In one embodiment of the copolymer of Formula (IIa), the sum of x and y is an integer from about 635 to about 714, n is an integer from about 114 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 556 to about 635, n is an integer from about 91 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 476 to about 556, n is an integer from about 80 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 397 to about 476, n is an integer from about 68 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 317 to about 397, n is an integer from about 57 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 238 to about 317, n is an integer from about 45 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 159 to about 238, n is an integer from about 34 to about 227, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 119 to about 159, n is an integer from about 23 to about 170, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 79 to about 119, n is an integer from about 23 to about 114, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is an integer from about 40 to about 79, n is an integer from about 23 to about 57, and the ratio of the sum of x and y to n is from about 0.7 to about 7. In a further embodiment, the ratio of the sum of x and y to n is from about 1.4 to about 5.2. In a further embodiment, the ratio of the sum of x and y to n is from about 1.7 to about 3.5.

In one embodiment of the copolymer of Formula (IIa), the sum of x and y is about 155, and y is about 36% of the sum of x and y. In a further embodiment, R2 is hydrogen. In a further embodiment, n is about 45.

### Copolymer Synthesis

The copolymers disclosed herein may be prepared by reacting a polycarboxylic acid or polycarboxylic acid-PEG, e.g., a polyamino acid such as poly(L-glutamic acid) represented by Formula (III),

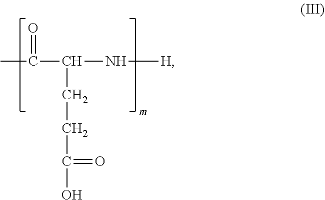

or a polyamino acid-PEG such as methoxy-poly(ethylene glycol)-block-poly(L-glutamic acid) represented by Formula (V),

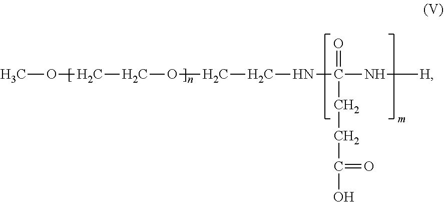

with an alkylamine, such as N-decylamine represented by Formula (IV),

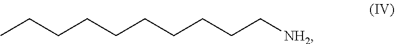

in the presence of a coupling agent, such as N,N′-diisopropylcarbodiimide (DIC), dicyclohexylcarbodiimide (DCC), 1-ethyl-3-(3-dimethylaminopropyl)carbodiimide (EDC), (1-[bis(dimethylamino)methylene]-1H-1,2,3-triazolo[4,5-b]pyridinium 3-oxide hexafluorophosphate (HATU), 3-[bis(dimethylamino)methyliumyl]-3H-benzotriazol-1-oxide hexafluorophosphate (HBTU), O-(1H-6-chlorobenzotriazole-1-yl)-1,1,3,3-tetramethyluronium hexafluorophosphate (HCTU), benzotriazole-1-yl-oxy-tris-(dimethylamino)-phosphonium hexafluorophosphate (BOP), 1-[(1-(cyano-2-ethoxy-2-oxoethylideneaminooxy) dimethylaminomorpholino)] uronium hexafluorophosphate (COMU), 3-(diethoxy-phosphoryloxy)-1,2,3-benzo[d]triazin-4(3H)-one e (DEPBT), (7-azabenzotriazol-1-yloxy)trispyrrolidinophosphonium hexafluorophosphate (PyAOP), benzotriazole-1-yl-oxy-tris-pyrrolidino-phosphonium hexafluorophosphate (PyBOP), 1-cyano-2-ethoxy-2-oxoethylideneaminooxy-tris-pyrrolidino-phosphonium hexafluorophosphate (PyOxim), N,N,N′,N′-tetramethyl-O-(N-succinimidyl)uronium tetrafluoroborate, O-[N-succinimidyl)-1,1,3,3-tetramethyluronium tetrafluoroborate (TSTU), and 6-chloro-benzotriazole-1-yloxy-tris-pyrrolidinophosphonium hexafluorophosphate (PyClock). The molar ratio of polycarboxylic acid or polycarboxylic acid-PEG to alkylamine can be varied to achieve a desirable y/(x+y).

For example, to prepare the copolymer of Formula (I) wherein P is a random copolymer moiety comprising x units of the formula

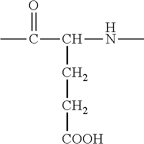

and y units of the formula

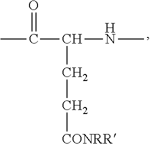

one of R and R′ is linear C₁₀ alkyl and the other hydrogen, and R² is hydrogen, a poly(L-glutamic acid) of Formula (III) is reacted with N-decylamine of Formula (IV) in the presence of a coupling agent mentioned above, wherein m is equal to the sum of x and y, and the ratio of the product of the number of moles of the poly(L-glutamic acid) of Formula (III) multiplied by m to the number of moles of the compound of Formula (IV) is from about 1:0.1 to about 1:0.9. In a further embodiment, the ratio of the product of the number of moles of the poly(L-glutamic acid) of Formula (III) multiplied by m to the number of moles of the compound of Formula (IV) is from about 1:0.2 to about 1:0.8. In a further embodiment, the ratio of the product of the number of moles of the poly(L-glutamic acid) of Formula (III) multiplied by m to the number of moles of the compound of Formula (IV) is from about 1:0.4 to about 1:0.7. In a further embodiment, the ratio of the product of the number of moles of the poly(L-glutamic acid) of Formula (III) multiplied by m to the number of moles of the compound of Formula (IV) is about 1:0.6. In one embodiment, the coupling agent is DIC.

As another example, to prepare the copolymer of Formula (IIa) as defined above, with R² being hydrogen, a methoxy-poly(ethylene glycol)-block-poly(L-glutamic acid) of Formula (V) is reacted with N-decylamine of Formula (IV) in the presence of a coupling agent mentioned above, wherein m is equal to the sum of x and y, n is as defined in Formula (IIa), and the ratio of the product of the number of moles of the compound of Formula (V) multiplied by m to the number of moles of the compound of Formula (IV) is from about 1:0.1 to about 1:0.9. In a further embodiment, the ratio of the product of the number of moles of the compound of Formula (V) multiplied by m to the number of moles of the compound of Formula (IV) is from about 1:0.2 to about 1:0.8. In a further embodiment, the ratio of the product of the number of moles of the compound of Formula (V) multiplied by m to the number of moles of the compound of Formula (IV) is from about 1:0.4 to about 1:0.7. In a further embodiment, the ratio of the product of the number of moles of the compound of Formula (V) multiplied by m to the number of moles of the compound of Formula (IV) is about 1:0.6. In one embodiment, the coupling agent is DIC.

### Pharmaceutical Compositions

In another aspect, the present disclosure relates to a pharmaceutical composition comprising nanoparticles comprising a glycopeptide antibiotic complexed with one or more copolymers disclosed herein and a charge neutral polymer selected from the group consisting of polylactic acid, polylactic acid-polyethylene glycol (PLA-PEG), poly(lactic-co-glycolic acid), a polyester-polyethylene glycol (polyester-PEG), and a combination thereof. The glycopeptide antibiotic is positively charged at a pH of from about 6 to about 8.

In one embodiment, the pharmaceutical composition further comprises a base selected from the group consisting of an Arrhenius base, a Brønsted-Lowry base, and a combination thereof, to balance extra negative charges on the copolymers disclosed herein and stabilize the positive charged glycopeptide antibiotic in the nanoparticles. Exemplary bases include ammonium hydroxide, hexyl amine, and decyl amine.

In one embodiment of the pharmaceutical composition, the polyester-PEG is a polylactone family polymer-PEG selected from the group consisting of polylactide-PEG, polyglycolide-PEG, poly(lactic-co-glycolic acid)-PEG, polypropiolactone-PEG, polybutyrolactone-PEG, polyvalerolactone-PEG, polycaprolactone-PEG, polyheptalactone-PEG, polyoctalactone-PEG, polynonalactone-PEG, polydecalactine-PEG, polyundecalactine-PEG, and polydodecalactine-PEG.

In one embodiment of the pharmaceutical composition, the charge neutral polymer is polylactic acid-PEG, and the one or more copolymers does not include a PEG moiety. In a further embodiment, the one or more copolymers which does not include a PEG moiety is a copolymer of Formula (I).

In one embodiment of the pharmaceutical composition, the charge neutral polymer is polylactic acid-PEG and the one or more copolymers is a copolymer of Formula (II) or (IIa). In a further embodiment, the one or more copolymers is a copolymer of Formula (IIa).

In one embodiment of the pharmaceutical composition, the charge neutral polymer is PLA-PEG and the glycopeptide antibiotic is complexed with at least two copolymers, wherein one of the at least two copolymers does not include a PEG moiety and the other a copolymer of Formula (II) or (IIa). In one further embodiment, the one of the at least two copolymers which does not include a PEG moiety is a copolymer of Formula (I). In another further embodiment, the other of the at least two copolymers is a copolymer of Formula (IIa).

In one embodiment of the pharmaceutical composition, the charge neutral polymer is a combination of PLA-PEG and polylactic acid, and the one or more copolymers does not include a PEG moiety. In a further embodiment, the one or more copolymers which does not include a PEG moiety is a copolymer of Formula (I).

In one embodiment of the pharmaceutical composition, the charge neutral polymer is a combination of PLA-PEG and polylactic acid, and the one or more copolymers is a copolymer of Formula (II) or (IIa). In a further embodiment, the one or more copolymers is a copolymer of Formula (IIa).

In one embodiment of the pharmaceutical composition, the charge neutral polymer is a combination of PLA-PEG and polylactic acid, and the glycopeptide antibiotic is complexed with at least two copolymers, wherein one of the at least two copolymers does not include a PEG moiety and the other a copolymer of Formula (II) or (IIa). In one further embodiment, the one of the at least two copolymers which does not include a PEG moiety is a copolymer of Formula (I). In another further embodiment, the other of the at least two copolymers is a copolymer of Formula (IIa).

In one embodiment of the pharmaceutical composition, the charge neutral polymer is polylactic acid, and the one or more copolymers is a copolymer of Formula (II) or (IIa). In a further embodiment, the one or more copolymers is a copolymer of Formula (IIa).

In one embodiment of the pharmaceutical composition, the nanoparticles have an average diameter of from about 60 nm to about 260 nm as measured by dynamic light scattering. In a further embodiment, the nanoparticles have an average diameter of from about 80 nm to about 230 nm. In a further embodiment, the nanoparticles have an average diameter of from about 100 nm to about 200 nm. In a further embodiment, the nanoparticles have an average diameter of from about 120 nm to about 180 nm. In a further embodiment, the nanoparticles have an average diameter of from about 140 nm to about 160 nm.

In one embodiment of the pharmaceutical composition, a targeting moiety is attached to the nanoparticles for targeted delivery of the nanoparticles to a bacterial biofilm or the surface of a bacterium. In one embodiment, the targeting moiety is capable of binding to and targeting a methicillin-resistant Staphylococcus aureus (MRSA) biofilm. In another embodiment, the targeting moiety is capable of binding to and hence targeting a biofilm comprising Streptococcal bacteria, such as Streptococcus mutans and Streptococcus pyogenes. In another embodiment, the targeting moiety is capable of targeting Staphylococcal bacteria via binding to the surface of the Staphylococcal bacteria.

In one embodiment, the targeting moiety is an antibody capable of binding to a bacterial biofilm or the surface of a bacterium, for example, an anti-polysaccharide intercellular adhesin (PIA) antibody, or an anti-wall teichoic acid (WTA) antibody. A partially deacetylated poly-β(1-6)-N-acetylglucosamine (PNAG), PIA is an extracellular polysaccharide essential for staphylococcal biofilm formation. Suitable anti-PIA antibodies for use as a targeting moiety include a commercially available anti-PNAG therapeutical antibody (SAR279356) (Cat #TAB-799CL) from Creative Biolabs (Shirley, NY, USA). Teichoic acids (TA) are polysaccharides residing within the cell wall of Gram-positive bacteria such as Staphylococcus aureus. Wall teichoic acids (WTA) are teichoic acids covalently linked to the peptidoglycan (PDG) layer of the cell wall. Suitable anti-WTA antibodies for use as a targeting moiety include anti-WTA monoclonal antibodies disclosed in U.S. Pat. Nos. 9,803,002 and 9,884,126, and International Application Publication WO 2014/194247, each of which is incorporated herein by reference in its entirety for all purposes.

In another embodiment, the targeting moiety is a peptide that binds to poly-N-acetyl glucosamine (PNAG) or deacetylated PNAG (dPNAG) of bacteria such as Staphylococcus. Exemplary peptides are disclosed in International Application Publication WO 2005/103084, incorporated herein by reference in its entirety for all purposes.

In another embodiment, the targeting moiety is a peptide that binds to a biofilm comprising bacteria such as Streptococcus mutans, and Streptococcus pyogenes. Exemplary peptides are disclosed in International Application Publication WO2010091294, incorporated herein by reference in its entirety for all purposes.

The targeting moiety may be attached to the nanoparticles either covalently or non-covalently. Strategies for covalent attachment include, for example, carbodiimide chemistry, maleimide chemistry or click chemistry. Strategies for non-covalent attachment include, for example, physical adsorption, ionic binding, adapter binding, and binding of biotin to avidin or streptavidin.

In one embodiment, the targeting moiety is attached to the charge neutral polymer of the nanoparticles. In a further embodiment, the charge neutral polymer is PLA-PEG, a polyester-PEG, or a combination thereof, and the targeting moiety is attached to the PEG moiety of the charge neutral polymer. In still a further embodiment, the targeting moiety is attached to the PEG moiety of PLA-PEG. Charge neutral PEG-based amphiphilic block copolymers, such as PLA-PEG and a polyester-PEG, form nanoparticles by self-assembly in water. The attachment of the targeting moiety to a charge neutral PEG-based amphiphilic block copolymer can be accomplished, for example, by adding to the end of the PEG block of the charge neural polymer a functional group, such as azide, amine, maleimide, N-hydroxysuccinimide (NHS), dibenzocyclooctyne (DBCO), biotin, or avidin, depending on the attachment strategy. Upon self-assembly of the charge neutral polymer in water, the functional group is on the hydrophilic shell of the nanoparticles accessible for a compatible functional group on the targeting moiety to react to form a covalent bond, or to bind via non-covalent interaction.

In one embodiment of the pharmaceutical composition, the glycopeptide antibiotic for use is selected from the group consisting of A477, A35512, A40926, A41030, A42867, A47934, A80407, A82846, A83850, A84575, AB-65, actaplanin, actinoidin, ardacin, avoparcin, azureomycin, chloroeremomycin, chloroorienticin, chloropolysporin, dalbavancin, decaplanin, N-demethylvancomycin, eremomycin, galacardin, helvecardin, izupeptin, kibdelin, LL-AM374, mannopeptin, MM45289, MM47761, MM47766, MM55266, MM55270, OA-7653, orienticin, oritavancin, parvodicin, ristocetin, ristomycin, synmonicin, teicoplanin, telavancin, UK-68597, UK-69542, UK-72051, vancomycin, a pharmaceutically acceptable salt thereof, and a combination of the foregoing. In another embodiment, the glycopeptide antibiotic is selected from the group consisting of vancomycin, oritavancin, telavancin, dalbavancin, and a combination thereof. In a further embodiment, the glycopeptide antibiotic is oritavancin, telavancin, or vancomycin. In another embodiment, the glycopeptide antibiotic is vancomycin. In another embodiment, the glycopeptide antibiotic is oritavancin.

In one embodiment of the pharmaceutical composition, the glycopeptide antibiotic for use is one of the compounds described in International Application Publication No. WO 2018/217800, WO 2018/217808, WO 2020/106787, and WO 2020/106791, the disclosure of each of which is incorporated herein by reference in their entireties for all purposes.

In one embodiment of the pharmaceutical composition, the glycopeptide antibiotic for use is a compound of Formula (VI), (VII), or (VIII), or a pharmaceutically acceptable salt thereof, as described herein.

Formula (VI)

Glycopeptide-R¹ (VI),

wherein,
R¹ is conjugated at a primary amine group of the Glycopeptide;
R¹ is —(CH₂)ₙ₁—C(O)—O—(CH₂)ₙ₂—CH₃; —(CH₂)ₙ₁—C(O)—NH—(CH₂)ₙ₂—CH₃; —C(O)—(CH₂)ₙ₂—CH₃; —(CH₂)ₙ₁—NH—C(O)—(CH₂)ₙ₂—CH₃; —(CH₂)ₙ₁—O—C(O)—(CH₂)ₙ₂—CH_{3;} —(CH₂)ₙ₁—O—C(O)—NH—(CH₂)ₙ₂—CH₃; —(CH₂)ₙ₁—O—(CO)—O—(CH₂)ₙ₂—CH₃ or —(CH₂)ₙ₁—NH—C(O)—O—(CH₂)ₙ₂—CH₃;
n1 is 1, 2, 3,4 or 5; and
n2 is 6, 7, 8, 9, 10, 11, 12, 13, 14 or 15.

In another embodiment of the pharmaceutical composition comprising a compound of Formula (VI), the compound of Formula (VI) is a compound of Formula (VII), or a pharmaceutically acceptable salt thereof:

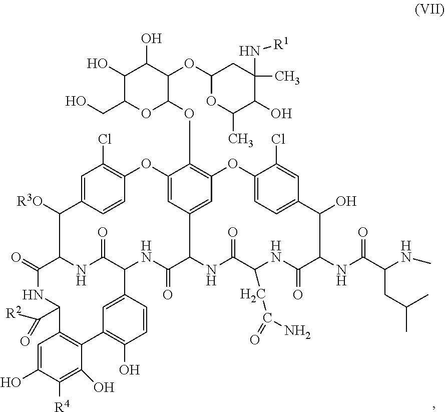

wherein,
R¹ is —(CH₂)ₙ₁—C(O)—O—(CH₂)ₙ₂—CH₃; —(CH₂)_{n 1}—C(O)—NH—(CH₂)ₙ₂—CH₃; —C(O)—(CH₂)ₙ₂—CH₃; —(CH₂)ₙ₁—NH—C(O)—(CH₂)ₙ₂—CH₃; —(CH₂)ₙ₁—O—C(O)—(CH₂)ₙ₂—CH_{3;} —(CH₂)ₙ₁—O—C(O)—NH—(CH₂)ₙ₂—CH₃; —(CH₂)ₙ₁—O—(CO)—O—(CH₂)ₙ₂—CH₃ or
—(CH₂)ₙ₁—NH—C(O)—O—(CH₂)ₙ₂—CH₃;
n1 is 1, 2, 3,4 or 5;
n2 is 6, 7, 8, 9, 10, 11, 12, 13, 14 or 15;
R² is OH or NH—(CH₂)_{q}—R⁵;
q is 1, 2, 3, 4, or 5;
R³ is H or

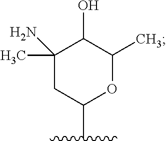 [SMILES: *C1CC(C)(N)C(O)C(C)O1]

R⁴ is H or CH₂—NH—CH₂—PO₃H₂; and
R⁵ is —N(CH₃)₂, —N⁺(CH₃)₃, —N⁺(CH₃)ₙ(n-C₁₄H₂₉), or

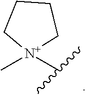 [SMILES: *[N+]1(C)CCCC1]

In one embodiment, the glycopeptide antibiotic for use in the pharmaceutical composition is a compound of Formula (VIII), or a pharmaceutically acceptable salt thereof:

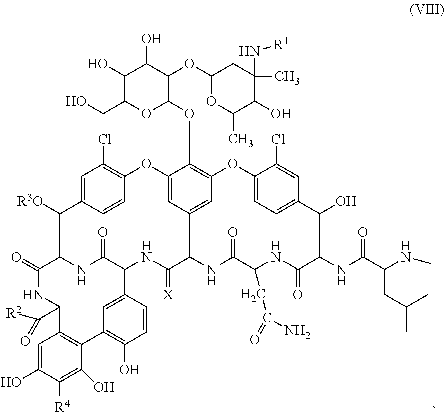

wherein,
R¹ is C₁-C₁₈ linear alkyl, C₁-C₁₈ branched alkyl, R⁵—Y—R⁶—(Z)ₙ, or

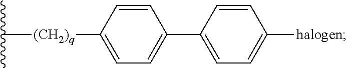

R² is —OH or —NH—(CH₂)_{q}—R⁷;
R³ is H or

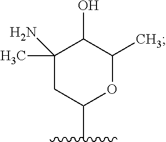 [SMILES: *C1CC(C)(N)C(O)C(C)O1]

R⁴ is H or CH₂—NH—CH₂—PO₃H₂;
n is 1 or 2;
q is 1, 2, 3, 4, or 5;
X is O, S, or NH;
each Z is independently selected from the group consisting of hydrogen, aryl, cycloalkyl, cycloalkenyl, heteroaryl and heterocyclic;
R⁵ and R⁶ are each independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl;
R⁷ is —N(CH₂)₂; —N⁺(CH₂)₃; or

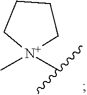 [SMILES: *[N+]1(C)CCCC1]

Y is selected from the group consisting of oxygen, sulfur, —S—S—, —NR⁸—, —S(O)—, —SO₂—, —NR⁸C(O)—, —OSO₂—, —OC(O)—, —NR⁸SO₂—, —C(O)NR⁸—, —C(O)O—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸, —OC(O)O—, —NR⁸C(O)O—, —NR⁸C(O)NR⁸, —OC(O)NR⁸— and —NR⁸SO₂NR⁸—;
each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

### Formula (VI) Specifics

In one embodiment of the pharmaceutical composition comprising a compound of Formula (VI), the glycopeptide is vancomycin, telavancin, chloroeremomycin or decaplanin. In a further embodiment, the glycopeptide is telavancin, chloroeremomycin or decaplanin.

The structures of hundreds of natural and semisynthetic glycopeptides have been determined. These structures are highly related and fall within five structural subtypes, I-V, and the present disclosure is not limited to a particular subtype, so long as the glycopeptide includes a primary amine group to conjugate the R¹ group. Of the varying structural subtypes, type I structures contain aliphatic chains, whereas types II, III, and IV include aromatic side chains within these amino acids. Unlike types I and II, types III and IV contain an extra F-O-G ring system. Type IV structures have, in addition, a long fatty-acid chain attached to the sugar moiety. Structures of type V, such as complestatin, chloropeptin I, and kistamincin A and B, contain the characteristic tryptophan moiety linked to the central amino acid.

In one embodiment of the pharmaceutical composition comprising a compound of Formula (VI), the glycopeptide is one of the glycopeptides described in International Application Publication No. WO 2014/085526, the disclosure of which is incorporated by reference herein for all purposes.

In one embodiment of the pharmaceutical composition comprising a compound of Formula (VI), the glycopeptide is A477, A35512, A40926, A41030, A42867, A47934, A80407, A82846, A83850, A84575, AB-65, actaplanin, actinoidin, ardacin, avoparcin, azureomycin, chloroorienticin, chloropolysporin, chloroeremomycin, decaplanin, N-demethylvancomycin, eremomycin, galacardin, helvecardin A, helvecardin B, izupeptin, kibdelin, LL-AM374, mannopeptin, MM45289, MM47761, MM47766. MM55266, MM55270, OA-7653, orienticin, parvodicin, ristocetin, ristomycin, synmonicin, teicoplanin, telavancin, UK-68597, UK-69542, UK-72051, vancomycin, or a pharmaceutically acceptable salt of one of the foregoing.

In one embodiment of the pharmaceutical composition comprising a compound of Formula (VI), the glycopeptide is vancomycin. In another embodiment, the glycopeptide is telavancin. In another embodiment, the glycopeptide is chloroeremomycin. In another embodiment, the glycopeptide is decaplanin.

In one embodiment of the pharmaceutical composition comprising a compound of Formula (VI), n1 is 1, 2 or 3; and n2 is 8, 9, 10, 11 or 12. In another embodiment, n1 is 2 and n2 is 10. In another embodiment, n1 is 1 and n2 is 9. In a further embodiment, the glycopeptide is vancomycin, telavancin or chloroeremomycin. In a further embodiment, the glycopeptide is vancomycin.

In one embodiment of the pharmaceutical composition comprising a compound of Formula (VI), R¹ is —(CH₂)ₙ₁—C(O)—O—(CH₂)ₙ₂—CH₃. In a further embodiment, n1 is 1, 2 or 3; and n2 is 8, 9, 10, 11 or 12. In even a further embodiment, n1 is 2 and n2 is 10. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—C(O)—NH—(CH₂)ₙ₂—CH₃. When R¹ is so defined, in one embodiment, n1 is 2 or 3; and n2 is 8, 9, 10, 11 or 12. In another embodiment, n1 is 2 and n2 is 10. In still another embodiment, n1 is 1 and n2 is 9. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—NH—C(O)—(CH₂)ₙ₂—CH₃. In a further embodiment, n1 is 1, 2 or 3; and n2 is 8, 9, 10, 11 or 12. In even a further embodiment, n1 is 2 and n2 is 10. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—O—C(O)—(CH₂)ₙ₂—CH₃. In a further embodiment, n1 is 2 or 3; and n2 is 8, 9, 10, 11 or 12. In even a further embodiment, n1 is 2 and n2 is 10. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —C(O)—(CH₂)ₙ₂—CH₃. In a further embodiment, n2 is 8, 9, 10, 11 or 12. In even a further embodiment, n2 is 10. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), n1 is 1, 2 or 3; and n2 is 10, 11, 12 or 13 in. In even a further embodiment, n1 is 2 and n2 is 10 or 11. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—C(O)—O—(CH₂)ₙ₂—CH₃. In a further embodiment, n1 is 1, 2 or 3; and n2 is 10, 11, 12 or 13. In even a further embodiment, n1 is 2 and n2 is 10 or 11. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—C(O)—NH—(CH₂)ₙ₂—CH₃. When R¹ is so defined, in one embodiment, n1 is 2 or 3; and n2 is 10, 11, 12 or 13. In another embodiment, n1 is 1, 2 or 3 and n2 is 10 or 11. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—NH—C(O)—(CH₂)ₙ₂—CH₃. In a further embodiment, n1 is 1, 2 or 3; and n2 is 10, 11, 12 or 13. In even a further embodiment, n1 is 2 and n2 is 10 or 11. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —(CH₂)ₙ₁—O—C(O)—(CH₂)ₙ₂—CH₃. In a further embodiment, n1 is 1, 2 or 3; and n2 is 10, 11, 12 or 13. In even a further embodiment, n1 is 2 and n2 is 10 or 11. In a further embodiment, the glycopeptide is vancomycin, telavancin or chloroeremomycin. In even a further embodiment, the glycopeptide is vancomycin.

In one embodiment of the pharmaceutical composition comprising a compound of Formula (VI), R¹ is —C(O)—(CH₂)ₙ₂—CH₃. In a further embodiment, n2 is 10, 11, 12 or 13. In even a further embodiment, n2 is 10 or 11. In a further embodiment, the glycopeptide is vancomycin, telavancin or chloroeremomycin. In even a further embodiment, the glycopeptide is vancomycin.

In yet another embodiment of the pharmaceutical composition comprising a compound of Formula (VI), one or more hydrogen atoms of the compound of Formula (VI) are replaced with a deuterium atom. For example, in one embodiment, R³ and/or R⁴ is deuterium.

In another embodiment of the pharmaceutical composition comprising a compound of Formula (VI), the compound of Formula (VI) is a compound of Formula (VII), or a pharmaceutically acceptable salt thereof. Formula (VII) is defined above.

### Formula (VII) Specifics

In one embodiment of the pharmaceutical composition comprising a compound of Formula (VII), R² is OH. In a further embodiment, R⁴ is H.

In one embodiment of the pharmaceutical composition comprising a compound of Formula (VII), R² is OH. In a further embodiment, R⁴ is CH₂—NH—CH₂—PO₃H₃.

In one embodiment of the pharmaceutical composition comprising a compound of Formula (VII), R² is —NH—(CH₂)₃—R⁵. In a further embodiment, R³ and R⁴ are H.

In one embodiment of the pharmaceutical composition comprising a compound of Formula (VII), R² is —NH—(CH₂)₃—R⁵. In a further embodiment, R⁴ is CH₂—NH—CH₂—PO₃H₂.

In one embodiment of the pharmaceutical composition comprising a compound of Formula (VII), R² is —NH—(CH₂)_{q}—R⁵. In a further embodiment, R² is —NH—(CH₂)₃—N(CH₃)₂. In another embodiment of the pharmaceutical composition comprising a compound of Formula (VII), R² is —NH—(CH₂)₃—N⁺(CH₃)₃. In yet another embodiment of the pharmaceutical composition comprising a compound of Formula (VII), R² is —NH—(CH₂)₃—N⁺(CH₃)₂(n-C₁₄H₂₉). In a further embodiment, R² is

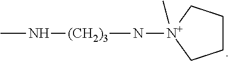

In one embodiment of the pharmaceutical composition comprising a compound of Formula (VII), R² is —NH—(CH₂)_{q}—N(CH₃)₂. In another embodiment, R² is —NH—(CH₂)_{q}—N⁺(CH₃)₃. In another embodiment, R² is —NH—(CH₂)_{q}—R⁵ and R⁵ is —N⁺(CH₃)₂(n-C₁₄H₂₉). In yet another embodiment, R² is —NH—(CH₂)_{q}—R⁵ and R⁵ is

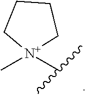 [SMILES: *[N+]1(C)CCCC1]

In one embodiment of the pharmaceutical composition comprising a compound of Formula (VII), R¹ is —(CH₂)ₙ₁—O—C(O)—(CH₂)ₙ₂—CH₃ or —(CH₂)ₙ₁—NH—C(O)—(CH₂)ₙ₂—CH₃. In a further embodiment, R² is OH, R³ is H and R⁴ is H. In even a further embodiment, n1 is 1, 2 or 3, n2 is 9, 10, 11, 12, 13 or 14. In even a further embodiment, n1 is 2 and n2 is 10. In yet even a further embodiment, R¹ is —(CH₂)ₙ₁—O—C(O)—(CH₂)ₙ₂—CH₃.

In one embodiment of the pharmaceutical composition comprising a compound of Formula (VII), R¹ is —(CH₂)ₙ₁—NH—C(O)—(CH₂)ₙ₂—CH₃. In a further embodiment, R² is OH, R³ is H and R⁴ is H. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—NH—C(O)—(CH₂)ₙ₂—CH₃, R² is OH, R³ is H, R⁴ is H, n1 is 2 and n2 is 10, i.e., the compound of Formula (VII), is of the following formula, referred to as “RV62” herein:

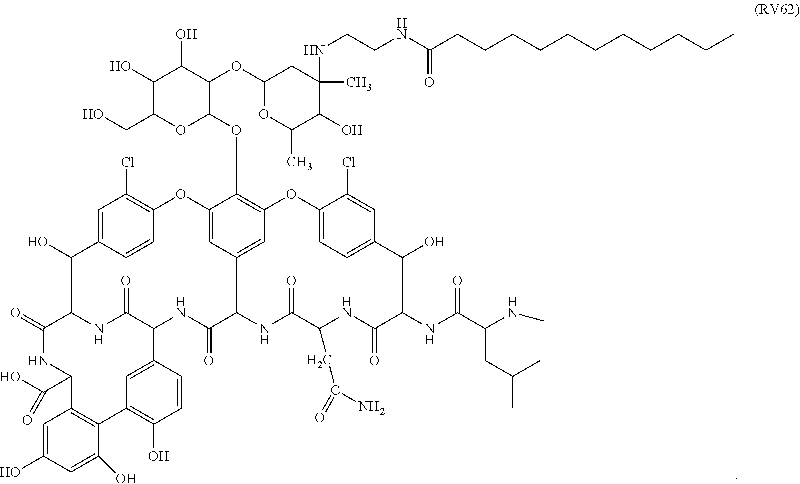 [SMILES: CCCCCCCCCCCC(=O)NCCNC1(C)CC(OC2C(Oc3c4cc5cc3Oc3ccc(cc3Cl)C(O)C3NC(=O)C(NC(=O)C5NC(=O)C(CC(N)=O)NC(=O)C(NC(=O)C(CC(C)C)NC)C(O)c5ccc(c(Cl)c5)O4)c4ccc(O)c(c4)-c4c(O)cc(O)cc4C(C(=O)O)NC3=O)OC(CO)C(O)C2O)OC(C)C1O]

In one embodiment of the pharmaceutical composition comprising a compound of Formula (VII), R¹ is —(CH₂)ₙ₁—O—C(O)—(CH₂)ₙ₂—CH₃. In a further embodiment, R² is OH, R³ is H and R⁴ is H. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—C(O)—O—(CH₂)ₙ₂—CH₃. In a further embodiment, R² is OH, R³ is H and R⁴ is H. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—C(O)—NH—(CH₂)ₙ₂—CH₃. In a further embodiment, R² is OH, R³ is H and R⁴ is H. In a further embodiment, n1 is 1, 2 or 3, n2 is 9, 10, 11, 12, 13 or 14. In another embodiment, n1 is 2 and n2 is 10. In still another embodiment, n1 is 1 and n2 is 9.

In one embodiment of the pharmaceutical composition comprising a compound of Formula (VII), R¹ is —(CH₂)ₙ₁—C(O)—NH—(CH₂)ₙ₂—CH₃, R² is OH, R³ is H, R⁴ is H, n1 is 1 and n2 is 9, i.e., the compound of Formula (VII), has the following structure, referred to herein as “RV94”.

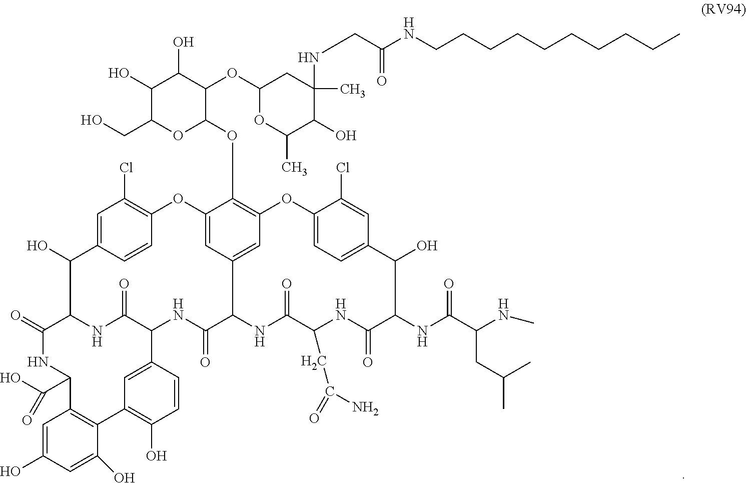 [SMILES: CCCCCCCCCCNC(=O)CNC1(C)CC(OC2C(Oc3c4cc5cc3Oc3ccc(cc3Cl)C(O)C3NC(=O)C(NC(=O)C5NC(=O)C(CC(N)=O)NC(=O)C(NC(=O)C(CC(C)C)NC)C(O)c5ccc(c(Cl)c5)O4)c4ccc(O)c(c4)-c4c(O)cc(O)cc4C(C(=O)O)NC3=O)OC(CO)C(O)C2O)OC(C)C1O]

In one embodiment of the pharmaceutical composition comprising a compound of Formula (VII), R¹ is —C(O)—(CH₂)ₙ₂—CH₃. In a further embodiment, R² is OH and R³ and R⁴ are H. In a further embodiment, n2 is 9, 10, 11, 12, 13 or 14. In even a further embodiment, n2 is 10.

In another embodiment of the pharmaceutical composition comprising a compound of Formula (VII), R¹ is —(CH₂)ₙ₁—O—C(O)—(CH₂)ₙ₂—CH₃ or —(CH₂)ₙ₁—NH—C(O)—(CH₂)ₙ₂—CH₃. In a further embodiment, R² is OH, R³ is

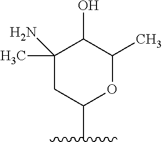

and R⁴ is H. In even a further embodiment, n1 is 1, 2 or 3, n2 is 10, 11, 12, 13 or 14. In even a further embodiment, n1 is 2 and n2 is 10. In yet even a further embodiment, R¹ is —(CH₂)ₙ₁—O—C(O)—(CH₂)ₙ₂—CH₃.

In another embodiment of the pharmaceutical composition comprising a compound of Formula (VII), R¹ is —(CH₂)ₙ₁—NH—C(O)—(CH₂)ₙ₂—CH₃. In a further embodiment, R² is OH, R³ is

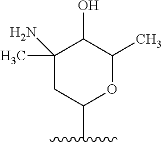

and R⁴ is H. In even a further embodiment, n1 is 1, 2 or 3, n2 is 9, 10, 11, 12, 13 or 14. In even a further embodiment, n1 is 2 and n2 is 10.

In yet another embodiment of the pharmaceutical composition comprising a compound of Formula (VII), R¹ is —(CH₂)ₙ₁—O—C(O)—(CH₂)ₙ₂—CH₃. In a further embodiment, R² is OH, R³ is

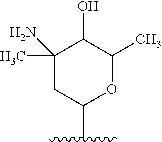

and R⁴ is H. In even a further embodiment, n1 is 1, 2 or 3, n2 is 9, 10, 11, 12, 13 or 14. In even a further embodiment, n1 is 2 and n2 is 10.

In yet another embodiment of the pharmaceutical composition comprising a compound of Formula (VII), R¹ is —(CH₂)ₙ₁—C(O)—O—(CH₂)ₙ₂—CH₃. In a further embodiment, R² is OH, R³ is

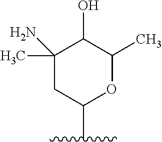

and R⁴ is H. In even a further embodiment, n1 is 1, 2 or 3, n2 is 9, 10, 11, 12, 13 or 14. In even a further embodiment, n1 is 2 and n2 is 10.

In yet another embodiment of the pharmaceutical composition comprising a compound of Formula (VII), R¹ is —(CH₂)ₙ₁—C(O)—NH—(CH₂)ₙ₂—CH₃. In a further embodiment, R² is OH, R³ is

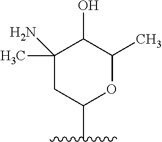

and R⁴ is H. In even a further embodiment, n1 is 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —C(O)—(CH₂)ₙ₂—CH₃. In a further embodiment, R² is OH, R³ is

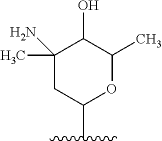

and R⁴ is H. In even a further embodiment, n2 is 9, 10, 11, 12, 13 or 14. In even a further embodiment, n2 is 10.

In one embodiment of the pharmaceutical composition comprising a compound of Formula (VII), R¹ is —(CH₂)ₙ₁—O—C(O)—(CH₂)ₙ₂—CH₃ or —(CH₂)ₙ₁—NH—C(O)—(CH₂)ₙ₂—CH₃. In a further embodiment, R² is OH, R³ is H and R⁴ is CH₂—NH—CH₂—PO₃H₂. In even a further embodiment, n1 is 1, 2 or 3, n2 is 9, 10, 11, 12, 13 or 14. In even a further embodiment, n1 is 2 and n2 is 10. In yet even a further embodiment, R¹ is —(CH₂)ₙ₁—O—C(O)—(CH₂)ₙ₂—CH₃.

In one embodiment of the pharmaceutical composition comprising a compound of Formula (VII), R¹ is —(CH₂)ₙ₁—NH—C(O)—(CH₂)ₙ₂—CH₃. In a further embodiment, R² is OH, R³ is H and R⁴ is CH₂—NH—CH₂—PO₃H₂. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—O—C(O)—(CH₂)ₙ₂—CH₃. In a further embodiment, R² is OH, R³ is H and R⁴ is CH₂—NH—CH₂—PO₃H₂. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—C(O)—O—(CH₂)ₙ₂—CH₃. In a further embodiment, R² is OH, R³ is H and R⁴ is CH₂—NH—CH₂—PO₃H₂. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —(CH₂)ₙ₁—C(O)—NH—(CH₂)ₙ₂—CH₃. In a further embodiment, R² is OH, R³ is H and R⁴ is CH₂—NH—CH₂—PO₃H₂. In even a further embodiment, n1 is 1, 2 or 3, n2 is 9, 10, 11, 12, 13 or 14. In even a further embodiment, n1 is 2 and n2 is 10.

In one embodiment of the pharmaceutical composition comprising a compound of Formula (VII), R¹ is —C(O)—(CH₂)ₙ₂—CH₃. In a further embodiment, R² is OH, R³ is H and R⁴ is CH₂—NH—CH₂—PO₃H₂. In even a further embodiment, n2 is 9, 10, 11, 12, 13 or 14. In even a further embodiment, n2 is 10.

In one embodiment of the pharmaceutical composition comprising a compound of Formula (VII), R¹ is —(CH₂)ₙ₁—O—C(O)—(CH₂)ₙ₂—CH₃ or —(CH₂)ₙ₁—NH—C(O)—(CH₂)ₙ₂—CH₃. In a further embodiment, R² is —NH—(CH₂)_{q}—R⁵, R³ is H and R⁴ is H. In even a further embodiment, n1 is 1, 2 or 3, n2 is 9, 10, 11, 12, 13 or 14. In even a further embodiment, n1 is 2 and n2 is 10. In yet even a further embodiment, R¹ is —(CH₂)ₙ₁—O—C(O)—(CH₂)ₙ₂—CH₃. In yet even a further embodiment, q is 2 or 3 and R⁵ is N(CH₃)₂.

In one embodiment of the pharmaceutical composition comprising a compound of Formula (VII), R¹ is —(CH₂)ₙ₁—NH—C(O)—(CH₂)ₙ₂—CH₃. In a further embodiment, R² is —NH—(CH₂)_{q}—R⁵, R³ and R⁴ are H. In even a further embodiment, n1 is 1, 2 or 3, n2 is 9, 10, 11, 12, 13 or 14. In even a further embodiment, n is 2 and n2 is 10. In yet even a further embodiment, q is 2 or 3 and R⁵ is N(CH₃)₂.

In one embodiment of the pharmaceutical composition comprising a compound of Formula (VII), R¹ is —(CH₂)ₙ₁—O—C(O)—(CH₂)ₙ₂—CH₃. In a further embodiment, R² is —NH—(CH₂)_{q}—R⁵, R³ and R⁴ are H. In even a further embodiment, n1 is 1, 2 or 3, n2 is 9, 10, 11, 12,13 or 14. In even a further embodiment, n1 is 2 and n2 is 10. In yet even a further embodiment, q is 2 or 3 and R⁵ is N(CH₃)₂.

In one embodiment of the pharmaceutical composition comprising a compound of Formula (VII), R¹ is —(CH₂)ₙ₁—C(O)—O—(CH₂)ₙ₂—CH₃. In a further embodiment, R² is —NH—(CH₂)_{q}—R⁵, R³ and R⁴ are H. In even a further embodiment, n1 is 1, 2 or 3, n2 is 9, 10, 11, 12, 13 or 14. In even a further embodiment, n1 is 2 and n2 is 10. In yet even a further embodiment, q is 2 or 3 and R⁵ is N(CH₃)₂.

In one embodiment of the pharmaceutical composition comprising a compound of Formula (VII), R¹ is —(CH₂)ₙ₁—C(O)—NH—(CH₂)ₙ₂—CH₃. In a further embodiment, R² is —NH—(CH₂)_{q}—R⁵, R³ is H and R⁴ is H. In even a further embodiment, n1 is 1, 2 or 3, n2 is 9, 10, 11, 12, 13 or 14. In even a further embodiment, n1 is 2 and n2 is 10. In yet even a further embodiment, q is 2 or 3 and R⁵ is N(CH₃)₂.

In one embodiment of the pharmaceutical composition comprising a compound of Formula (VII), R¹ is —C(O)—(CH₂)ₙ₂—CH₃. In a further embodiment, R² is —NH—(CH₂)_{q}—R⁵, R³ is H and R⁴ is H. In even a further embodiment, n2 is 9, 10, 11, 12, 13 or 14. In even a further embodiment, n2 is 10. In yet even a further embodiment, q is 2 or 3 and R⁵ is N(CH₃)₂.

In yet another embodiment of the disclosed pharmaceutical composition, one or more hydrogen atoms of a compound Formula (VII) or a pharmaceutically acceptable salt thereof are replaced with a deuterium atom, for example, R³ and/or R⁴ is deuterium.

The compounds of Formulae (VI) and (VII) can be prepared according to methods and steps known to those of ordinary skill in the art. For example, the compounds may be prepared according to methods described in U.S. Pat. No. 6,392,012; U.S. Patent Application Publication Nos. 2017/0152291 and 2016/0272682, and International Application Publication Nos. WO 2018/08197 and WO 2018/217808, each of which is hereby incorporated by reference in their entirety for all purposes.

Pharmaceutical compositions provided herein can also include a compound of Formula (VIII), or a pharmaceutically acceptable salt thereof.

### Formula (VIII) Specifics

In one embodiment of the pharmaceutical composition comprising a compound of Formula (VIII), Y of is selected from the group consisting of oxygen, sulfur, —S—S—, —NR⁸—, —S(O)—, —SO₂—, —OSO₂—, —NR⁸SO₂—, —SO₂NR⁸—, —SO₂O—, —P(O)(OR⁸)O—, —P(O)(OR⁸)NR⁸, —OP(O)(OR⁸)O—, —OP(O)(OR⁸)NR⁸—, NR⁸C(O)NR⁸—, and —NR⁸SO₂NR⁸—.

In one embodiment of the pharmaceutical composition comprising a compound of Formula (VIII), R¹ does not include an amide or ester moiety.

In one embodiment of the pharmaceutical composition comprising a compound of Formula (VIII), R¹ is R⁵—Y—R⁶—(Z)ₙ. In a further embodiment, R⁵ is —(CH₂)₂—, R⁶ is —(CH₂)₁₀—, Z is hydrogen, and n is 1. In a further embodiment, X is O. In a further embodiment, Y is NR⁸. In a further embodiment, R⁸ is hydrogen. In another embodiment, R¹ is —(CH₂)₂—NH—(CH₂₉—CH₃.

In another embodiment of the pharmaceutical composition comprising a compound of Formula (VIII), R¹ is —(CH₂)₂—NH—(CH₂)₉—CH₃, X is O, R² is OH and R³ and R⁴ are H, i.e., the compound of Formula (VIII), is of the following formula, which is referred to as “RV40” herein.

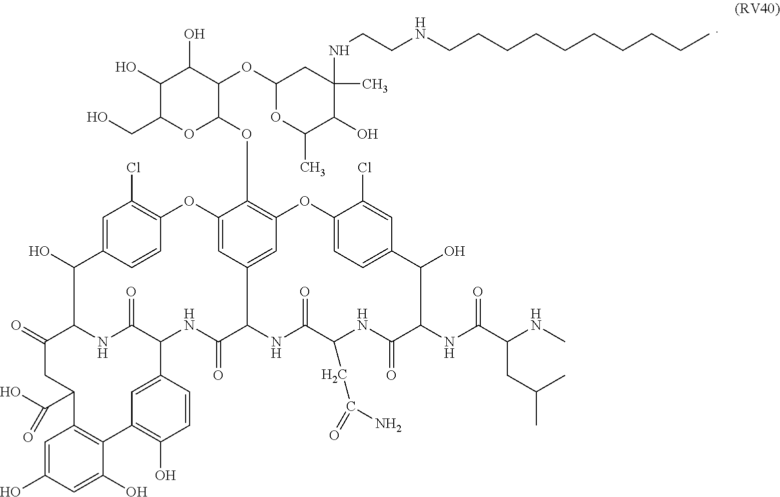 [SMILES: CCCCCCCCCCNCCNC1(C)CC(OC2C(Oc3c4cc5cc3Oc3ccc(cc3Cl)C(O)C(NC(=O)C(CC(C)C)NC)C(=O)NC(CC(N)=O)C(=O)NC5C(=O)NC3C(=O)NC(C(=O)CC(C(=O)O)c5cc(O)cc(O)c5-c5cc3ccc5O)C(O)c3ccc(c(Cl)c3)O4)OC(CO)C(O)C2O)OC(C)C1O]

In one embodiment of the pharmaceutical composition comprising a compound of Formula (VIII), R¹ is —CH₂—NH—(CH₂)₁₀—CH₃. In a further embodiment, X is O, R² is OH and R³ and R⁴ are H.

In one embodiment of the pharmaceutical composition comprising a compound of Formula (VIII), R¹ is —(CH₂)₂—NH—(CH₂)₁₀—CH₃. In a further embodiment, X is O, R² is OH and R³ and R⁴ are H.

In one embodiment of the pharmaceutical composition comprising a compound of Formula (VIII), R¹ is —(CH₂)₂—NH—(CH₂)₁₁—CH₃. In a further embodiment, X is O, R² is OH and R³ and R⁴ are H.

In one embodiment of the pharmaceutical composition comprising a compound of Formula (VIII), R¹ is

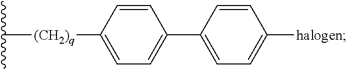

X is O; and R² is —NH—(CH₂)_{q}—R⁷. In a further embodiment, R² is —NH—(CH₂)₃—R⁷. In a further embodiment, R¹ is

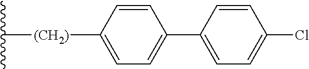

and R⁷ is —N⁺(CH₂)₃ or —N(CH₂)₂.

In one embodiment of the pharmaceutical composition comprising a compound of Formula (VIII), R¹ is C₁₀-C₁₆ alkyl. In a further embodiment, R¹ is C₁₀ alkyl.

In one embodiment of the pharmaceutical composition comprising a compound of Formula (VIII), R² is OH, R³ and R⁴ are H and X is O. In a further embodiment, R¹ is

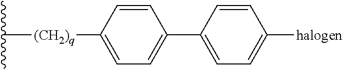

or R⁵—Y—R⁶—(Z)ₙ. In even a further embodiment, R¹ is R⁵—Y—R⁶-(Z)ₙ, R⁵ is methylene, ethylene or propylene; R⁶ is —(CH₂)₉—, —(CH₂)₁₀—, —(CH₂)₁₁—, or —(CH₂)₁₂—, Z is H and n is 1. In even a further embodiment, R⁵ is —(CH₂)₂—, R⁶ is —(CH₂)₁₀—, Y is NR⁸. In even a further embodiment, R⁸ is hydrogen.

In one embodiment of the pharmaceutical composition comprising a compound of Formula (VIII), the compound is one of the compounds provided in Table 1 below. It should be noted that the compound can also be provided as a pharmaceutically acceptable salt. The compounds in Table 1 are identified by their respective R¹, R² and X groups. Compounds of Table 1, in a further embodiment, are defined as having R³ and R⁴ as both H. In another embodiment, R³ is

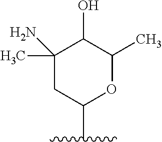

and R⁴ is H in each compound of Table 1. In yet another embodiment, R³ is H and R⁴ is CH₂—NH—CH₂—PO₃H₂ in each compound of Table 1. In even another embodiment, R³ is

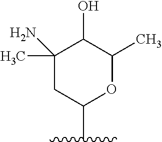

and R⁴ is CH₂—NH—CH₂—PO₃H₂ in each compound of Table 1.

TABLE 1
Exemplary compounds of Formula (VIII) for use in a pharmaceutical composition of the invention

| Compound | | | |
|---|---|---|---|
| # | R² | X | R¹ |
| 1. | OH | O | —(CH₂)₅—CH₃ (n-hexyl) |
| 2. | OH | O | —(CH₂)₆—CH₃ (n-heptyl) |
| 3. | OH | O | —(CH₂)₇—CH₃ (n-octyl) |
| 4. | OH | O | —(CH₂)₈—CH₃ (n-nonyl) |
| 5. | OH | O | —(CH₂)₉—CH₃ (n-decyl) |
| 6. | OH | O | —(CH₂)₁₀—CH₃ (n-undecyl) |
| 7. | OH | O | —(CH₂)₁₁—CH₃ (n-dodecyl) |
| 8. | OH | O | —(CH₂)₁₂—CH₃ (n-tridecyl) |
| 9. | OH | O | —(CH₂)₁₃—CH₃ (n-butadecyl) |
| 10. | OH | O | —(CH₂)₁₄—CH₃ (n-pentadecyl) |
| 11. | OH | O | —(CH₂)₁₅—CH₃ (n-hexadecyl) |
| 12. | OH | O | —(CH₂)₁₆—CH₃ (n-heptadecyl) |
| 13. | OH | O | —(CH₂)₁₇—CH₃ (n-octadecyl) |
| 14. | OH | O | —CH₂—NH—(CH₂)₅—CH₃ |
| 15. | OH | O | —(CH₂)₂—NHSO₂—(CH₂)₅—CH₃ |
| 16. | OH | O | —(CH₂)₂—NHSO₂—(CH₂)₆—CH₃ |
| 17. | OH | O | —(CH₂)₂—NHSO₂—(CH₂)₇—CH₃ |
| 18. | OH | O | —(CH₂)₂—NHSO₂—(CH₂)₈—CH₃ |
| 19. | OH | O | —(CH₂)₂—NHSO₂—(CH₂)₉—CH₃ |
| 20. | OH | O | —(CH₂)₂—NHSO₂—(CH₂)₁₀—CH₃ |
| 21. | OH | O | —(CH₂)₂—NHSO₂—(CH₂)₁₁—CH₃ |
| 22. | OH | O | —(CH₂)₂—NHSO₂—(CH₂)₁₂—CH₃ |
| 23. | OH | O | —(CH₂)₂—OSO₂—(CH₂)₅—CH₃ |
| 24. | OH | O | —(CH₂)₂—OSO₂—(CH₂)₆—CH₃ |
| 25. | OH | O | —(CH₂)₂—OSO₂—(CH₂)₇—CH₃ |
| 26. | OH | O | —(CH₂)₂—OSO₂—(CH₂)₈—CH₃ |
| 27. | OH | O | —(CH₂)₂—OSO₂—(CH₂)₉—CH₃ |
| 28. | OH | O | —(CH₂)₂—OSO₂—(CH₂)₁₀—CH₃ |
| 29. | OH | O | —(CH₂)₂—OSO₂—(CH₂)₁₁—CH₃ |
| 30. | OH | O | —(CH₂)₂—OSO₂—(CH₂)₁₂—CH₃ |
| 31. | OH | O | —(CH₂)₂—OSO₂—(CH₂)₁₃—CH₃ |
| 32. | OH | O | —(CH₂)₂—OSO₂—(CH₂)₁₄—CH₃ |
| 33. | OH | O | —(CH₂)₂—NH—(CH₂)₂—CH₃ |
| 34. | OH | O | —(CH₂)₂—NH—(CH₂)₃—CH₃ |
| 35. | OH | O | —(CH₂)₂—NH—(CH₂)₄—CH₃ |
| 36. | OH | O | —(CH₂)₂—NH—(CH₂)₅—CH₃ |
| 37. | OH | O | —(CH₂)₂—NH—(CH₂)₆—CH₃ |
| 38. | OH | O | —(CH₂)₂—NH—(CH₂)₇—CH₃ |
| 39. | OH | O | —(CH₂)₂—NH—(CH₂)₈—CH₃ |
| 40. | OH | O | —(CH₂)₂—NH—(CH₂)₉—CH₃ |
| 41. | OH | O | —(CH₂)₂—NH—(CH₂)₁₀—CH₃ |
| 42. | OH | O | —(CH₂)₂—NH—(CH₂)₁₁—CH₃ |
| 43. | OH | O | —(CH₂)₂—NH—(CH₂)₁₂—CH₃ |
| 44. | OH | O | —(CH₂)₂—NH—(CH₂)₁₃—CH₃ |
| 45. | OH | O | —(CH₂)₂—NH—(CH₂)₁₄—CH₃ |
| 46. | OH | O | —(CH₂)₂—OC(O)—(CH₂)₅—CH₃ |
| 47. | OH | O | —(CH₂)₂—OC(O)—(CH₂)₆—CH₃ |
| 48. | OH | O | —(CH₂)₂—OC(O)—(CH₂)₇—CH₃ |
| 49. | OH | O | —(CH₂)₂—OC(O)—(CH₂)₈—CH₃ |
| 50. | OH | O | —(CH₂)₂—OC(O)—(CH₂)₉—CH₃ |
| 51. | OH | O | —(CH₂)₂—OC(O)—(CH₂)₁₀—CH₃ |
| 52. | OH | O | —(CH₂)₂—OC(O)—(CH₂)₁₁—CH₃ |
| 53. | OH | O | —(CH₂)₂—OC(O)—(CH₂)₁₂—CH₃ |
| 54. | OH | O | —(CH₂)₂—OC(O)—(CH₂)₁₃—CH₃ |
| 55. | OH | O | —(CH₂)₂—C(O)O—(CH₂)₅—CH₃ |
| 56. | OH | O | —(CH₂)₂—C(O)O—(CH₂)₆—CH₃ |
| 57. | OH | O | —(CH₂)₂—C(O)O—(CH₂)₇—CH₃ |
| 58. | OH | O | —(CH₂)₂—C(O)O—(CH₂)₈—CH₃ |
| 59. | OH | O | —(CH₂)₂—C(O)O—(CH₂)₉—CH₃ |
| 60. | OH | O | —(CH₂)₂—C(O)O—(CH₂)₁₀—CH₃ |
| 61. | OH | O | —(CH₂)₂—C(O)O—(CH₂)₁₁—CH₃ |
| 62. | OH | O | —(CH₂)₂—C(O)O—(CH₂)₁₂—CH₃ |
| 63. | OH | O | —(CH₂)₂—NHSO₂—(CH₂)₅—CH₃ |
| 64. | OH | O | —(CH₂)₂—NHSO₂—(CH₂)₆—CH₃ |
| 65. | OH | O | —(CH₂)₂—NHSO₂—(CH₂)₇—CH₃ |
| 66. | OH | O | —(CH₂)₂—NHSO₂—(CH₂)₈—CH₃ |
| 67. | OH | O | —(CH₂)₂—NHSO₂—(CH₂)₉—CH₃ |
| 68. | OH | O | —(CH₂)₂—NHSO₂—(CH₂)₁₀—CH₃ |
| 69. | OH | O | —(CH₂)₂—NHSO₂—(CH₂)₁₁—CH₃ |
| 70. | OH | O | —(CH₂)₂—NHSO₂—(CH₂)₁₂—CH₃ |
| 71. | OH | O | —(CH₂)₂—NHC(O)—(CH₂)₅—CH₃ |
| 72. | OH | O | —(CH₂)₂—NHC(O)—(CH₂)₆—CH₃ |
| 73. | OH | O | —(CH₂)₂—NHC(O)—(CH₂)₇—CH₃ |
| 74. | OH | O | —(CH₂)₂—NHC(O)—(CH₂)₈—CH₃ |
| 75. | OH | O | —(CH₂)₂—NHC(O)—(CH₂)₉—CH₃ |
| 76. | OH | O | —(CH₂)₂—NHC(O)—(CH₂)₁₀—CH₃ |
| 77. | OH | O | —(CH₂)₂—NHC(O)—(CH₂)₁₁—CH₃ |
| 78. | OH | O | —(CH₂)₂—NHC(O)—(CH₂)₁₂—CH₃ |
| 79. | OH | O | 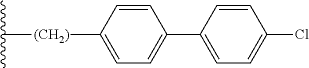 |
| 80. | OH | O | —(CH₂)₂—OC(O)—(CH₂)₆—CH₃ |
| 81. | OH | O | —(CH₂)₂—OC(O)—(CH₂)₇—CH₃ |
| 82. | OH | O | —(CH₂)₂—OC(O)—(CH₂)₈—CH₃ |
| 83. | OH | O | —(CH₂)₂—OC(O)—(CH₂)₉—CH₃ |
| 84. | OH | O | —(CH₂)₂—OC(O)—(CH₂)₁₀—CH₃ |
| 85. | OH | O | —(CH₂)₂—OC(O)—(CH₂)₁₁—CH₃ |
| 86. | OH | O | —(CH₂)₂—OC(O)—(CH₂)₁₂—CH₃ |
| 87. | OH | O | —(CH₂)₂—C(O)NH—(CH₂)₅—CH₃ |
| 88. | OH | O | —(CH₂)₂—C(O)NH—(CH₂)₆—CH₃ |
| 89. | OH | O | —(CH₂)₂—C(O)NH—(CH₂)₇—CH₃ |
| 90. | OH | O | —(CH₂)₂—C(O)NH—(CH₂)₈—CH₃ |
| 91. | OH | O | —(CH₂)₂—C(O)NH—(CH₂)₉—CH₃ |
| 92. | OH | O | —(CH₂)₂—C(O)NH—(CH₂)₁₀—CH₃ |
| 93. | OH | O | —(CH₂)₂—C(O)NH—(CH₂)₁₁—CH₃ |
| 94. | OH | O | —(CH₂)₂—C(O)NH—(CH₂)₁₂—CH₃ |
| 95. | OH | O | —(CH₂)₂—S—(CH₂)₅—CH₃ |
| 96. | OH | O | —(CH₂)₂—S—(CH₂)₆—CH₃ |
| 97. | OH | O | —(CH₂)₂—S—(CH₂)₇—CH₃ |
| 98. | OH | O | —(CH₂)₂—S—(CH₂)₈—CH₃ |
| 99. | OH | O | —(CH₂)₂—S—(CH₂)₉—CH₃ |
| 100. | OH | O | —(CH₂)₂—S—(CH₂)₁₀—CH₃ |
| 101. | OH | O | —(CH₂)₂—S—(CH₂)₁₁—CH₃ |
| 102. | OH | O | —(CH₂)₂—S—(CH₂)₁₂—CH₃ |
| 103. | OH | O | —(CH₂)₃—NH—(CH₂)₅—CH₃ |
| 104. | OH | O | —(CH₂)₃—NH—(CH₂)₆—CH₃ |
| 105. | OH | O | —(CH₂)₃—NH—(CH₂)₇—CH₃ |
| 106. | OH | O | —(CH₂)₃—NH—(CH₂)₈—CH₃ |
| 107. | OH | O | —(CH₂)₃—NH—(CH₂)₉—CH₃ |
| 108. | OH | O | —(CH₂)₃—NH—(CH₂)₁₀—CH₃ |
| 109. | OH | O | —(CH₂)₃—NH—(CH₂)₁₁—CH₃ |
| 110. | OH | O | —(CH₂)₃—NH—(CH₂)₁₂—CH₃ |
| 111. | OH | O | —(CH₂)₄—NH—(CH₂)₅—CH₃ |
| 112. | OH | O | —(CH₂)₄—NH—(CH₂)₆—CH₃ |
| 113. | OH | O | —(CH₂)₄—NH—(CH₂)₇—CH₃ |
| 114. | OH | O | —(CH₂)₄—NH—(CH₂)₈—CH₃ |
| 115. | OH | O | —(CH₂)₄—NH—(CH₂)₉—CH₃ |
| 116. | OH | O | —(CH₂)₄—NH—(CH₂)₁₀—CH₃ |
| 117. | OH | O | —(CH₂)₄—NH—(CH₂)₁₁—CH₃ |
| 118. | OH | O | —(CH₂)₄—NH—(CH₂)₁₂—CH₃ |
| 119. | OH | O | —(CH₂)₅—NH—(CH₂)₅—CH₃ |
| 120. | OH | O | —(CH₂)₅—NH—(CH₂)₆—CH₃ |
| 121. | OH | O | —(CH₂)₅—NH—(CH₂)₇—CH₃ |
| 122. | OH | O | —(CH₂)₅—NH—(CH₂)₈—CH₃ |
| 123. | OH | O | —(CH₂)₅—NH—(CH₂)₉—CH₃ |
| 124. | OH | O | —(CH₂)₅—NH—(CH₂)₁₀—CH₃ |
| 125. | OH | O | —(CH₂)₅—NH—(CH₂)₁₁—CH₃ |
| 126. | OH | O | —(CH₂)₅—NH—(CH₂)₁₂—CH₃ |
| 127. | OH | O | —(CH₂)₂—N[(CH₂)₉CH₃]₂ |
| 128. | OH | O | —(CH₂)₅—NH—(CH₂)₅—CH(CH₃)₂ |
| 129. | OH | O | —(CH₂)₅—NH—(CH₂)₆—CH(CH₃)₂ |
| 130. | OH | O | —(CH₂)₅—NH—(CH₂)₇—CH(CH₃)₂ |
| 131. | OH | O | —(CH₂)₅—NH—(CH₂)₈—CH(CH₃)₂ |
| 132. | OH | O | —(CH₂)₅—NH—(CH₂)₉—CH(CH₃)₂ |
| 133. | OH | O | —(CH₂)₅—NH—(CH₂)₁₀—CH(CH₃)₂ |
| 134. | OH | O | —(CH₂)₂—NH—CH₂—Ph |
| 135. | OH | O | —(CH₂)₂—NH—(CH₂)₂—Ph |
| 136. | OH | O | —(CH₂)₂—NH—(CH₂)₃—Ph |
| 137. | OH | O | —(CH₂)₂—NH—(CH₂)₄—Ph |
| 138. | OH | O | —(CH₂)₂—NH—(CH₂)₅—Ph |
| 139. | OH | O | —(CH₂)₂—NH—(CH₂)₆—Ph |
| 140. | OH | O | —(CH₂)₂—NH—(CH₂)₇—Ph |
| 141. | OH | O | —(CH₂)₂—NH—(CH₂)₈—Ph |
| 142. | OH | O | —(CH₂)₂—NH—CH₂-4-[(CH₃)₂CHCH₂—]Ph |
| 143. | OH | O | —(CH₂)₂—NH—CH₂-4-(Ph—S—)Ph |
| 144. | OH | O | —(CH₂)₂—NH—CH₂-4-(4-CF₃—Ph)Ph |
| 145. | OH | O | —(CH₂)₂—NH—CH₂-4-{4-[CH₃(CH₂)₄O—]—Ph}—Ph |
| 146. | OH | O | —(CH₂)₂—NH—CH₂-4-Cl—Ph |
| 147. | OH | O | —(CH₂)₂—NH—(CH₂)₂-4-Cl—Ph |
| 148. | OH | O | —(CH₂)₂—NH—(CH₂)₃-4-Cl—Ph |
| 149. | OH | O | —(CH₂)₂—NH—(CH₂)₄-4-Cl—Ph |
| 150. | OH | O | —(CH₂)₂—NH—(CH₂)₅-4-Cl—Ph |
| 151. | OH | O | —(CH₂)₂—NH—(CH₂)₆-4-Cl—Ph |
| 152. | OH | O | —(CH₂)₂—NH—(CH₂)₇-4-Cl—Ph |
| 153. | OH | O | —(CH₂)₂—NH—(CH₂)₈-4-Cl—Ph |
| 154. | OH | O | —(CH₂)₂—NH—CH₂-4-Ph—Ph |
| 155. | OH | O | —(CH₂)₂—NH—CH₂-4-(4-Cl—Ph)—Ph |
| 156. | OH | O | —(CH₂)₂—NH—CH₂-4-[CH₃(CH₂)₂O—]Ph |
| 157. | OH | O | —(CH₂)₂—NH—CH₂-4-[CH₃(CH₂)₃O—]Ph |
| 158. | OH | O | —(CH₂)₂—NH—CH₂-4-[CH₃(CH₂)₄O—]Ph |
| 159. | OH | O | —(CH₂)₂—NH—CH₂-4-[CH₃(CH₂)₅O—]Ph |
| 160. | OH | O | —(CH₂)₂—NH—CH₂-4-[CH₃(CH₂)₆O—]Ph |
| 161. | OH | O | —(CH₂)₂—NH—CH₂-4-[CH₃(CH₂)₇—O—]Ph |
| 162. | OH | O | —(CH₂)₂—NH—CH₂-4-[CH₃(CH₂)₈O—]Ph |
| 163. | OH | O | —(CH₂)₂—NH—CH₂-4-[CH₃(CH₂)₂—]Ph |
| 164. | OH | O | —(CH₂)₂—NH—CH₂-4-[CH₃(CH₂)₃—]Ph |
| 165. | OH | O | —(CH₂)₂—NH—CH₂-4-[CH₃(CH₂)₄—]Ph |
| 166. | OH | O | —(CH₂)₂—NH—CH₂-4-[CH₃(CH₂)₅—]Ph |
| 167. | OH | O | —(CH₂)₂—NH—CH₂-4-[CH₃(CH₂)₆—]Ph |
| 168. | OH | O | —(CH₂)₂—NH—CH₂-3-[Ph—S—]Ph |
| 169. | OH | O | —(CH₂)₂—NH—CH₂-4-[Ph—O—]Ph |
| 170. | OH | O | —(CH₂)₂—NH—CH₂-4-[CH₃CH₂Ph—O—]Ph |
| 171. | OH | O | —(CH₂)₂—NH—CH₂-4-[CH₃(CH₂)₂Ph—O—]Ph |
| 172. | OH | O | —(CH₂)₂—NH—CH₂-4-[CH₃(CH₂)₃Ph—O—]Ph |
| 173. | OH | O | —(CH₂)₂—NH—CH₂-4-[CH₃(CH₂)₄Ph—O—]Ph |
| 174. | OH | O | —(CH₂)₂—NH—CH₂-4-[CH₃(CH₂)₅Ph—O—]Ph |
| 175. | OH | O | —(CH₂)₂—NH—CH₂-4-[CH₃(CH₂)₆Ph—O—]Ph |
| 176. | OH | O | —(CH₂)₂—NH—CH₂-4-[CH₃(CH₂)₇Ph—O—]Ph |
| 177. | OH | O | —(CH₂)₂—NH—CH₂-4-[CH₃(CH₂)₈Ph—O—]Ph |
| 178. | OH | O | —(CH₂)₂—NH—CH₂-4-[CH₃(CH₂)₉Ph—O—]Ph |
| 179. | OH | O | —(CH₂)₂—NH—CH₂-3-[Ph—S—]Ph |
| 180. | OH | O | —(CH₂)₂—NH—CH₂-4-[Ph—S—]Ph |
| 181. | OH | O | —(CH₂)₂—NH—CH₂-cyclopropyl |
| 182. | OH | O | —(CH₂)₂—NH—(CH₂)₂-cyclopropyl |
| 183. | OH | O | —(CH₂)₂—NH—CH₂-cyclopentyl |
| 184. | OH | O | —(CH₂)₂—NH—(CH₂)₂-cyclopentyl |
| 185. | OH | O | —(CH₂)₂—NH—CH₂-cyclohexyl |
| 186. | OH | O | —(CH₂)₂—NH—(CH₂)₂-cyclohexyl |
| 187. | OH | O | —(CH₂)₂—NH—(CH₂)₈—CH═CH₂ |
| 188. | OH | O | —(CH₂)₂—NH—(CH₂)₈—CH(OH)—CH₃ |
| 189. | OH | O | —(CH₂)₂—NH—(CH₂)₃CH═CH(CH₂)₄CH₃ |
| | | | (trans) |
| 190. | OH | O | —(CH₂)₂—NH—CH₂CH═C(CH₃)(CH₂)₂—CH═C(CH₃)₂ |
| | | | (trans, trans) |
| 191. | OH | O | —(CH₂)₂—NHC(O)—(CH₂)₆—CH(CH₃)CH₃ |
| 192. | OH | O | —(CH₂)₂—S—(CH₂)₈Ph |
| 193. | OH | O | —(CH₂)₂NH—CH₂-4-[(CH₃)₃CO]—Ph |
| 194. | OH | O | —(CH₂)₂—S—(CHO)₃CH≡CH(CH₂)₄CH₃ |
| | | | (trans) |
| 195. | OH | O | —(CH₂)₂NH—CH₂-3,4-di—(CH₃CH₂O)—Ph |
| 196. | OH | O | —(CH₂)₂—S—CH₂CH₂(CF₂)₅CF₃ |
| 197. | OH | O | —(CH₂)₂NH—CH₂-4-[(CH₃)₂CH]—Ph |
| 198. | OH | O | —(CH₂)₂—S—CH₂-4-[(CH₃)₂CHCH₂—]Ph |
| 199. | OH | O | —(CH₂)₂—NH—CH₂-4-[CH₃[CH₂)₃C≡C]—Ph |
| 200. | OH | O | —(CH₂)₂—S—(CH₂)₁₁CH₃ |
| 201. | OH | O | —(CH₂)₂—NH—CH₂-4-[(CH₃)₂CHO]—Ph |
| 202. | OH | O | —(CH₂)₂—S—(CH₂) 8CH₃ |
| 203. | OH | O | —(CH₂)₂—NH—CH₂-4-(PhC—≡C)—Ph |
| 204. | OH | O | —(CH₂)₂—S—CH₂3,4-di-(PhCH₂O—)Ph |
| 205. | OH | O | —(CH₂)₂—NH—CH₂-4-[(CH₃)₃C]—Ph |
| 206. | OH | O | —(CH₂)₃—S—(CH₂)₈Ph |
| 207. | OH | O | —(CH₂)₂—NH—CH₂-5-(PhC≡C)-thiophen-2-yl |
| 208. | OH | O | —(CH₂)₃—S—(CH₂)₈CH₃ |
| 209. | OH | O | —(CH₂)₂—NH—CH₂-4-(PhCH≡CH—)Ph (trans) |
| 210. | OH | O | —(CH₂)₃—S—(CH₂)₉CH₃ |
| 211. | OH | O | —(CH₂)₂—NH—CH₂—(CH≡CH)₄—CH₃ |
| | | | (trans, trans, trans, trans) |
| 212. | OH | O | —(CH₂)₃—S—(CH₂)₆Ph |
| 213. | OH | O | —(CH₂)₂—N(C(O)Ph)—(CH₂)₉CH₃ |
| 214. | OH | O | —(CH₂)₄—S—(CH₂)₇CH₃ |
| 215. | OH | O | —(CH₂)₂—NH—CH₂-4-[4-(CH₃)₃C-thiazol-2-yl]—Ph |
| 216. | OH | O | —(CH₂)₂—S—(CH₂)₆Ph |
| 217. | OH | O | —(CH₂)₂—N[(CH₂)₉CH₃]—C(O)CHrS-4-pyridyl |
| 218. | OH | O | —(CH₂)₂—S—(CH₂)₁₀Ph |
| 219. | OH | O | —(CH₂)₂—N[(CH₂)₉CH₃]—C(O)-2-[PhCH(CH₃)NHC(O)—]Ph |
| | | | (R isomer) |
| 220. | OH | O | —(CH₂)₃—S—CH₂-4-[(CH₃)₂CHCH₂—]Ph |
| 221. | OH | O | —(CH₂)₂—N[(CH₂)₉CH₃]—C(O)-(1-PhCH₂OC(O)-2- |
| | | | oxoimidazolidin-5-yl) (S isomer) |
| 222. | OH | O | —(CH₂)₂—S—(CH₂)₃CH≡CH(CH₂)₄CH₃ |
| | | | (trans) |
| 223. | OH | O | —(CH₂)₂—N[(CH₂)₉CH₃]—C(O)-1-HO-cyclopropyl |
| 224. | OH | O | —(CH₂)₂—S—CH₂-4-[3,4-di-Cl—PhCH₂O—]Ph |
| 225. | OH | O | —(CH₂)₂—N(C(O)CH₂-naphth-2-yl)-(CH₂)₉CH₃ |
| 226. | OH | O | —(CH₂)₃—S—CH₂-4-[3,4-di-Cl—PhCH₂O—]Ph |
| 227. | OH | O | —(CH₂)₂—N[C(O)(CH₂)₉CH₂OH]—(CH₂)₉CH₃ |
| 228. | OH | O | —(CH₂)₂—SO-4-(4-Cl—Ph)—Ph |
| 229. | OH | O | —CH₂CH₂—N[C(O)CH₂(OCH₂CH₂)₂OCH₃]—(CH₂)₉CH₃ |
| 230. | OH | O | —(CH₂)₃—SO-4-(4-Cl—Ph)—Ph |
| 231. | OH | O | —(CH₂)₂—N[C(O)CH₂CH(Ph)₂]—(CH₂)₉CH₃ |
| 232. | OH | O | —(CH₂)₂—S—(CH₂)₁₀CH₃ |
| 233. | OH | O | —(CH₂)₂—N(C(O)CH₂-3-HO—Ph)—(CH₂)₉CH₃ |
| 234. | OH | O | —(CH₂)₃—S—(CH₂)₁₀CH₃ |
| 235. | OH | O | —(CH₂)₂—N(C(O)CH₂—NHC(O)-3-CH₃—Ph)—(CH₂)₉CH₃ |
| 236. | OH | O | —(CH₂)₃—S—CH₂-4-(CH₃(CH₂)₄O—]Ph |
| 237. | OH | O | —(CH₂)₂—N(C(O)CH₂CH₂—O—Ph)—(CH₂)₉CH₃ |
| 238. | OH | O | —(CH₂)₃—S—CH₂CH≡CH—CH≡CH(CH₂)₄CH₃ |
| | | | (trans, trans) |
| 239. | OH | O | —(CH₂)₂—N(C(O)CH₂CH₂-3-pyridyl)-(CH₂)₉CH₃ |
| 240. | OH | O | —(CH₂)₂—S—CHr4-[4-Cl—PhCH₂O—]Ph |
| 241. | OH | O | —(CH₂)₂—N(C(O)(CH₂)₃-4-CH₃O—Ph)—(CH₂)₉CH₃ |
| 242. | OH | O | —(CH₂)₃—S—CH₂4-[4-Cl—PhCH₂O—]Ph |
| 243. | OH | O | —(CH₂)₂—N(C(O)-indol-2-yl)-(CH₂)₉CH₃ |
| 244. | OH | O | —(CH₂)₃—S—CH₂-4-(4-CF₃—Ph—)Ph |
| 245. | OH | O | —(CH₂)₂—N{C(O)-1-[CH₃COC(O)—]-pyrrolidin-2-yl}-(CH₂)₉CH₃ |
| 246. | OH | O | —(CH₂)₃—S—CH₂-4-(4-F—PhSO₂NH—)Ph |
| 247. | OH | O | —(CH₂)₂—N(C(O)CH₂—NHC(O)—CH═CH-furan-2-yl)-(CH₂)₉CH₃ |
| | | | (trans) |
| 248. | OH | O | —(CH₂)₃—S—(CH₂)₈CH₃ |
| 249. | OH | O | —(CH₂)₂—N[C(O)-1-CH₃CHr7—CH₃-4-oxo-1,4- |
| | | | dihydro[1,8]naphthyridin-3-yl]-(CH₂)₉CH₃ |
| 250. | OH | O | —(CH₂)₃—S(O)—(CH₂)₆Ph |
| 251. | OH | O | —(CH₂)₂—N(C(O)-1,3-benzodioxol-5-yl)-(CH₂)₉CH₃ |
| 252. | OH | O | —(CH₂)₂—S(O)—(CH₂)₈Ph |
| 253. | OH | O | —(CH₂)₂—N(C(O)CH₂-4-oxo-2-thiooxothiazolidin-3-yl)-(CH₂)₉CH₃ |
| 254. | OH | O | —(CH₂)₂—S—(CH₂)₃-4-Cl—Ph |
| 255. | OH | O | —(CH₂)₂—N(C(O)-3,4,5-tri-HO-cyclohex-1-en-1-yl)- |
| | | | (CH₂)₉CH₃ (R,S,R isomer) |
| 256. | OH | O | —(CH₂)₂—S—(CH₂)c4—Cl—Ph |
| 257. | OH | O | —(CH₂)₂—N(C(O)CH₂CH₂C(O)NH₂)—(CH₂)₉CH₃ |
| 258. | OH | O | —(CH₂)₂—SO₂—(CH₂)₉CH₃ |
| 259. | OH | O | —(CH₂)₂—N(C(O)CH₂5—CH₃-2,4-dioxo-3,4- |
| | | | dihydropyrimidin-1-yl)-(CH2)₉CH₃ |
| 260. | OH | O | —(CH₂)₂—NHC(O)—CH₂CH(CH₂CH₂Ph)-{3-[4-(9H-fluroen-9- |
| | | | ylCH₂OC(O)NH(CH₂)₄—]-1,4-dioxohexahydro-1,2-α-pyrazin-2- |
| | | | yl}(S,S,S isomer) |
| 261. | OH | O | —(CH₂)₂—N(C(O)CH≡CH-imidazol-4-yl)-(CH₂)₉CH₃(trans) |
| 262. | OH | O | —(CH₂)₂—NHSO₂-4-(2-Cl—Ph)—Ph |
| 263. | OH | O | —(CH₂)₂—N[C(O)CH(CH₂CH₂C(O)NH₂)—NHC(O)O—CH₂Ph]—(CH₂)₉CH₃ |
| | | | (S isomer) |
| 264. | OH | O | —(CH₂)₂—NHSO₂-4-[4-(CH₃)₃C—Ph]—Ph |
| 265. | OH | O | —(CH₂)₂—N[C(O)CH(CH₂OH)NHC(O)O—CH₂Ph]—(CH₂)₉CH₃ |
| | | | (S isomer) |
| 266. | OH | O | —(CH₂)₂—NHSO₂-4-[4-(Ph)—Ph—]Ph |
| 267. | OH | O | —(CH₂)₂—N[C(O)CH[CH(OH)CH₃]NH—C(O)O—CH₂Ph]—(CH₂)₉CH₃ |
| | | | (S isomer) |
| 268. | OH | O | —(CH₂)₂—NH-4-(4-CF₃—Ph)—Ph |
| 269. | OH | O | —(CH₂)₂—N(C(O)CH₂NHSO₂-4-CH₃—Ph)—(CH₂)₉CH₃ |
| 270. | OH | O | —(CH₂)₂—N(C(O)CH(NH₂)CHr₃—HO—Ph)—(CH₂)₉CH₃ |
| 271. | OH | O | —(CH₂)₂—N(C(O)(CH₂)₃—NH₂)—(CH₂)₉CH₃ |
| 272. | OH | O | —(CH₂)₂—N(C(O)CH(NH₂)CH₃)—(CH₂)₉CH₃ |
| | | | (R isomer) |
| 273. | OH | O | —(CH₂)₂—N(C(O)-pyrrolidin-2-yl)-(CH₂)₉CH₃ |
| 274. | OH | O | —(CH₂)₂—N[C(O)CH(CH₂OH)NHC(O)—CH₃]—(CH₂)₉CH₃ |
| | | | (S isomer) |
| 275. | OH | O | —(CH₂)₂—N(C(O)-pyrrolidin-2-yl)-(CH₂)₉CH₃(R isomer) |
| 276. | OH | O | —(CH₂)₂—N[C(O)CH(NHC(O)CH₃)—(CH₂)₃—NHC(NH)NH₂]—(CH₂)₉CH₃ |
| | | | (S isomer) |
| 277. | OH | O | —(CH₂)₂—N(C(O)CH(NH₂)(CH₂)₄—NH₂)—(CH₂)₉CH₃ |
| | | | (S isomer) |
| 278. | OH | O | —(CH₂)₂—N(C(O)CH₂NHC(O)CH₃)—(CH₂)₉CH₃ |
| 279. | OH | O | —(CH₂)₂—N(C(O)-5-oxopyrrolidin-2-yl)-(CH₂)₉CH₃ |
| | | | (R isomer) |
| 280. | OH | O | —(CH₂)₂—N(C(O)CH(CH₃)OC(O)CH—(NH₂)CH₃)—(CH₂)₉CH₃ |
| | | | (R,R isomer) |
| 281. | —N(CH₂)₂ | O | 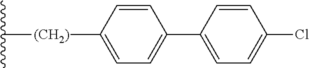 |
| 282. | —N(CH₂)₂ | NH | 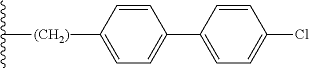 |
| 283. | —N(CH₂)₂ | S | 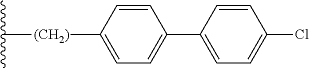 |
| 284. | —N(CH₂)₂ | H₂ | 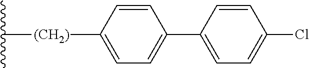 |
| 285. | 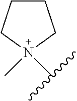 | O | 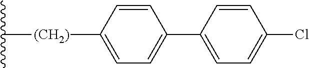 |
| 286. | 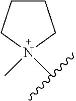 | NH | 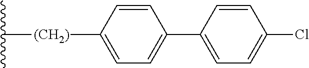 |
| 287. | 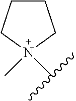 | S | 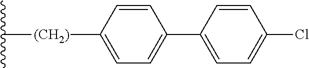 |
| 288. | 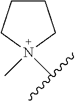 | H₂ | 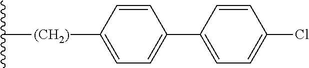 |
| 289. | —N⁺(CH₂)₃ | O | 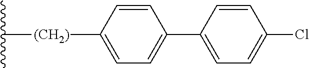 |
| 290. | —N⁺(CH₂)₃ | NH | 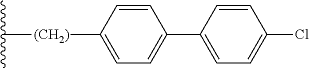 |
| 291. | —N⁺(CH₂)₃ | S | 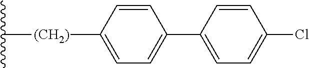 |
| 292. | —N⁺(CH₂)₃ | H₂ | 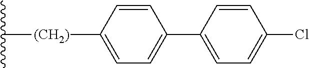 |
| 293. | OH | O | 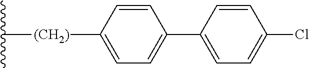 |
| 294. | OH | O | —(CH₂)₂—OC(O)—(CH₂)₅—CH₃ |

Ph—phenyl

In one embodiment of the pharmaceutical composition comprising a compound of Formula (VIII), the compound is Compound #40 of Table 1. In a further embodiment, R³ and R⁴ are each H in Compound #40.

In another embodiment of the pharmaceutical composition comprising a compound of Formula (VIII), the compound is Compound #79 of Table 1. In a further embodiment, R³ and R⁴ are each H in Compound #79.

In one embodiment of the pharmaceutical composition comprising a compound of Formula (VIII), one or more hydrogen atoms of the compound are replaced with a deuterium atom.

Compounds of Formula (VIII) are synthesized, in one embodiment, by the methods provided in U.S. Pat. Nos. 6,455,669, 7,160,984, 6,392,012; U.S. Patent Application Publication No. 2017/0152291; U.S. Patent Application Publication No. 2016/0272682, International Publication Nos. WO 2018/08197 and WO 2018/217800, the disclosure of each of which is incorporated by reference herein in their entireties for all purposes.

### Preparation of the Pharmaceutical Compositions

In still another aspect, the present disclosure relates to a method of preparing a pharmaceutical composition disclosed herein. The method includes:
(a) dissolving (i) a charge neutral polymer, (ii) a glycopeptide antibiotic, and (iii) one or more copolymers disclosed herein in an organic solvent to form an organic solution, and
(b) injecting the organic solution into water to form nanoparticles comprising the charge neutral polymer and the glycopeptide antibiotic complexed with the one or more copolymers.

In one embodiment, the organic solvent comprises one selected from the group consisting of dimethyl sulfoxide (DMSO), dimethylformamide (DMF), acetonitrile, acetone, benzyl alcohol, dichloromethane (DCM), ethylacetate, tetrahydrofuran (THF), chloroform, and a combination thereof. In another embodiment, the organic solvent comprises one selected from the group consisting of DMSO, acetonitrile, and a combination of DMSO and acetonitrile.

Alternatively, nanoparticles of the pharmaceutical composition may be prepared by using nanoprecipitation, wherein (i) a charge neutral polymer, (ii) a glycopeptide antibiotic, and (iii) one or more copolymers are dissolved in a 1:1 DMSO: acetonitrile. The nanoparticle suspension is produced by infusing about 3% to about 9%, about 5% to about 7%, or about 6% organic phase into aqueous phase through continuous flow nanoprecipitation process. The nanoparticle suspension is concentrated and most of the organic solvent is removed via diafiltration. Still alternatively, depending on the requirements for nanoparticles, such as particle size, particle size distribution and area of application, other techniques can be used to produce nanoparticles, such as solvent evaporation, salting-out, dialysis, supercritical fluid technology, microemulsion, nanoemulsion, surfactant-free emulsion, desolvation, ionic gelation, spray drying, freeze-drying and interfacial polymerization.

In another embodiment, the method further comprises adding a base selected from the group consisting of an Arrhenius base, a Brønsted-Lowry base, and a combination thereof. Exemplary bases for use include ammonium hydroxide, hexyl amine, and decyl amine. Without wishing to be bound by theory, the addition of the base balances extra negative charges on the copolymers and stabilize the positive charged glycopeptide antibiotic in the nanoparticles.

In another embodiment, the method further comprises attaching a targeting moiety to the nanoparticles. In a further embodiment, the charge neutral polymer of the nanoparticles is PLA-PEG, a polyester-PEG, or a combination thereof, and the attaching the targeting moiety to the nanoparticles comprises adding to the end of the PEG moiety of the neutral polymer a functional group configured for attaching the targeting moiety to the charge neutral polymer. In still a further embodiment, the charge neutral polymer of the nanoparticles to which the targeting moiety is attached is PLA-PEG. The functional group added to the end of the PEG moiety of the neutral polymer may vary depending on the attachment strategies and a compatible functional group on the targeting moiety. Exemplary functional groups include azide, amine, maleimide, NHS, DBCO, biotin, or avidin.

### Methods of Treatment

In still another aspect, the present disclosure relates to a method for treating a bacterial infection in a patient in need thereof. The method includes administering an effective amount of the pharmaceutical composition disclosed herein to the patient.

In one embodiment of the methods, the pharmaceutical composition comprises the glycopeptide antibiotic which is a compound of Formula (VI), (VII), or (VIII), or a pharmaceutically acceptable salt thereof. In another embodiment, the pharmaceutical composition comprises RV62, RV94, RV40, or any one of the compounds in Table 1. In another embodiment, the pharmaceutical composition comprises RV94. In another embodiment, the pharmaceutical composition comprises RV40. In another embodiment, the pharmaceutical composition comprises oritavancin. In a further embodiment, the bacterial infection treated by the methods is a methicillin-resistant Staphylococcus aureus (MRSA) infection. In a further embodiment, the MRSA infection is an MRSA biofilm infection.

In one embodiment of the methods, the bacterial infection is a pulmonary bacterial infection. In a further embodiment, the pulmonary bacterial infection is a pulmonary bacterial biofilm infection. With respect to pulmonary infections, the pharmaceutical compositions provided herein can be delivered to a patient in need of treatment via an inhalation delivery device that provides local administration to the site of infection. The inhalation delivery device employed in embodiments of the methods provided herein can be a nebulizer, a dry powder inhaler (DPI), or a metered dose inhaler (MDI), or any other suitable inhalation delivery device known to one of ordinary skill in the art. The device can contain and be used to deliver a single dose of the pharmaceutical composition, or the device can contain and be used to deliver multi-doses of the pharmaceutical composition disclosed herein. In one embodiment, the administering comprises administering to the lungs of the patient (pulmonary administration) via inhalation by using, for example, a nebulizer, a metered dose inhaler, or a dry powder inhaler.

In one embodiment of the methods, the administering comprises parenteral administration, e.g., intravenous administration or subcutaneous administration.

In one embodiment of the methods, the administering is carried out once daily. In another embodiment, the administering is carried out twice daily. In still another embodiment, the administering is carried out three or more times daily.

In one embodiment of the methods, the bacterial infection is a Gram-positive bacterial infection. In a further embodiment, the Gram-positive bacterial infection is a Gram-positive bacterial biofilm infection. The Gram-positive bacterial infection includes, but is not limited to, a Staphylococcus infection, a Streptococcus infection, an Enterococcus infection, a Bacillus infection, a Corynebaclerium infection, a Nocardia infection, a Clostridium, infection and a Listeria infection.

In one embodiment of the methods, the Gram-positive bacterial infection is a Gram-positive cocci infection. In a further embodiment, the Gram-positive cocci infection is a Streptococcus infection, an Enterococcus infection, a Staphylococcus infection, or a combination thereof.

In one embodiment of the methods, the Gram-positive cocci infection is a Streptococcus infection. In a further embodiment, the Streptococcus infection is a Streptococcus biofilm infection. In a further embodiment, the pharmaceutical composition administered according to the disclosed methods comprises RV94, RV40, or oritavancin. Streptococci are Gram-positive, non-motile cocci that divide in one plane, producing chains of cells. The primary pathogens include S. pyrogenes and S. pneumoniae but other species can be opportunistic. S. pyrogenes is the leading cause of bacterial pharyngitis and tonsillitis. It can also produce sinusitis, otitis, arthritis, and bone infections. Some strains prefer skin, producing either superficial (impetigo) or deep (cellulitis) infections. Streptoccocus pnemoniae is treated, in one embodiment, in a patient that has been diagnosed with community-acquired pneumonia or purulent meningitis.

S. pneumoniae is the major cause of bacterial pneumonia in adults, and in one embodiment, an infection due to S. pneumoniae is treated with the methods provided herein. Its virulence is dictated by its capsule. Toxins produced by streptococci include: streptolysins (S & O), NADase, hyaluronidase, streptokinase, DNAses, erythrogenic toxin (which causes scarlet fever rash by producing damage to blood vessels; requires that bacterial cells are lysogenized by phage that encodes toxin). Examples of Streptococcus infections treatable with the methods provided herein include, S. agalactiae, S. anginosus, S. bovis, S. canis, S. constellatus, S. dysgalactiae, S. equi, S. equinus, S. intermedins, S. mitis, S. mutans, S. oralis, S. parasanguinis, S. peroris, S. pneumoniae, S. pyogenes, S. ratti, S. salivarius, S. salivarius ssp. thermophilics, S. sanguinis, S. sobrinus, S. suis, S. uteris, S. vestibularis, S. viridans, and S. zooepidemicus infections.

In one embodiment of the methods, the Streptococcus infection is an S. agalactiae, S. anginosus, S. bovis, S. dysgalactiae, S. mitis, S. mutans, S. pneumoniae, S. pyogenes, S. sanguinis, or S. suis infection. In another embodiment, the Streptococcus infection is an S. mutans infection. In still another embodiment, the Streptococcus infection is an S. pneumoniae infection. In a further embodiment, the Streptococcus infection is a penicillin-intermediate S. pneumoniae (PISP) infection. In yet another embodiment, the Streptococcus infection is an S. dysgalactiae infection. In yet another embodiment, the Streptococcus infection is an S. pyogenes infection.

In one embodiment of the methods, the Gram-positive cocci infection is an Enterococcus infection. In a further embodiment, the Enterococcus infection is an Enterococcus biofilm infection. In a further embodiment, the pharmaceutical composition administered according to the disclosed methods comprises RV94, RV40, or oritavancin. In one embodiment, the Enterococcus infection is a vancomycin resistant Enterococcus infection (VRE). In another embodiment, the Enterococcus infection is a vancomycin sensitive Enterococcus infection (VSE).

The genus Enterococci includes Gram-positive, facultatively anaerobic organisms that are ovoid in shape and appear on smear in short chains, in pairs, or as single cells. Enterococci are important human pathogens that are increasingly resistant to antimicrobial agents. Examples of Enterococci treatable with the methods provided herein are E. avium, E. durans, E. faecalis, E. faecium, E. gallinarum, and E. solitarius. An Enterococcus species is treated, in one embodiment, in a patient that has been diagnosed with a urinary-catheter related infection.

In one embodiment of the methods, the Enterococcus infection is an Enterococcus faecalis (E. faecalis) infection.

In another embodiment of the methods, the Enterococcus infection is an Enterococcus faecium (E. faecium) infection.

In one embodiment of the method, the Enterococcus infection treated is resistant or sensitive to vancomycin or resistant or sensitive to penicillin. In a further embodiment, the Enterococcus infection is an E. faecalis or E. faecium infection. In a specific embodiment, the Enterococcus infection is an Enterococcus faecalis (E. faecalis) infection. In one embodiment, the E. faecalis infection is a vancomycin-sensitive E. faecalis infection. In another embodiment, the E. faecalis infection is a vancomycin-resistant E. faecalis infection. In yet another embodiment, the E. faecalis infection is an ampicillin-resistant E. faecalis infection. In another embodiment, the Enterococcus infection is an Enterococcus faecium (E. faecium) infection. In still another embodiment, the E. faecium infection is a vancomycin-resistant E. faecium infection. In yet a further embodiment, the E. faecium infection is a vancomycin-sensitive E. faecium infection. In still a further embodiment, the E. faecium infection is an ampicillin-resistant E. faecium infection.

In one embodiment of the methods, the Gram-positive cocci infection is a Staphylococcus infection. In a further embodiment, the Staphylococcus infection is a Staphylococcus biofilm infection. In a further embodiment, the pharmaceutical composition administered according to the disclosed methods comprises RV94, RV40, or oritavancin. Staphylococcus is Gram-positive non-motile bacteria that colonizes skin and mucus membranes. Staphylococci are spherical and occur in microscopic clusters resembling grapes. The natural habitat of Staphylococcus is nose; it can be isolated in 50% of normal individuals. 20% of people are skin carriers and 10% of people harbor Staphylococcus in their intestines. Examples of Staphylococci infections treatable with the methods provided herein include S. aureus, S. epidermidis, S. auricularis, S. carnosus, S. haemolyticus, S. hyicus, S. intermedius, S. lugdunensis, S. saprophytics, S. sciuri, S. simulans, and S. warneri infections.

In one embodiment of the methods, the Staphylococcal infection treated with the methods provided herein causes endocarditis or septicemia (sepsis). As such, the patient in need of treatment with the methods provided herein, in one embodiment, is an endocarditis patient. In another embodiment, the patient is a septicemia (sepsis) patient.

In one embodiment of the methods, the Staphylococcus infection is a Staphylococcus aureus (S. aureus) infection. In a further embodiment, the S. aureus infection is an S. aureus biofilm infection. In a further embodiment, the pharmaceutical composition administered according to the disclosed methods comprises RV94, RV40, or oritavancin. S. aureus colonizes mainly the nasal passages, but it may be found regularly in most anatomical locales, including skin oral cavity, and gastrointestinal tract. The S. aureus infection can be healthcare associated, i.e., acquired in a hospital or other healthcare setting, or community-acquired. In one embodiment, the S. aureus infection is a methicillin-resistant Staphylococcus aureus (MRSA) infection. In a further embodiment, the MRSA infection is an MRSA biofilm infection. In a further embodiment, the pharmaceutical composition administered according to the disclosed methods comprises RV94, RV40, or oritavancin. In another embodiment, the S. aureus infection is a methicillin-sensitive S. aureus (MSSA) infection. In another embodiment, the S. aureus infection is a vancomycin-intermediate S. aureus (VISA) infection. In a further embodiment, the S. aureus infection is an erythromycin-resistant (erm^{R}) vancomycin-intermediate S. aureus (VISA) infection. In still a further embodiment, the S. aureus infection is a heterogeneous vancomycin-intermediate S. aureus (hVISA) infection. In another embodiment, the S. aureus infection is a vancomycin-resistant S. aureus (VRSA) infection.

In another embodiment of the methods, the Staphylococcus infection is a Staphylococcus haemolyticus (S. haemolyticus) infection. In another embodiment, the Staphylococcus infection is a Staphylococcus epidermis (S. epidermis) infection. In a further embodiment, the Staphylococcus infection is an S. epidermidis coagulase-negative staphylococci (CoNS) infection. A Staphylococcus infection, e.g., an S. aureus infection, is treated in one embodiment, in a patient that has been diagnosed with mechanical ventilation-associated pneumonia.

In one embodiment of the methods, the Gram-positive cocci infection, e.g., a Streptococccus infection, an Enterococcus infection, or a Staphylococcus infection, is a penicillin resistant, methicillin resistant, or a vancomycin resistant bacterial infection. In a further embodiment, the resistant bacterial infection is a methicillin-resistant Staphylococcus infection, e.g., methicillin-resistant S. aureus (MRSA) or a methicillin-resistant Staphylococcus epidermidis (MRSE) infection. In another embodiment, the resistant bacterial infection is an oxacillin-resistant Staphylococcus (e.g., S. aureus) infection, a vancomycin-resistant Enterococcus infection or a penicillin-resistant Streptococcus (e.g., S. pneumoniae ) infection. In yet another embodiment, the Gram-positive cocci infection is an infection of vancomycin-resistant enterococci (VRE), vancomycin resistant Enterococcus faecium, which is also resistant to teicoplanin (VRE Fm Van A), vancomycin resistant Enterococcus faecium sensitive to teicoplanin (VRE Fm Van B), vancomycin resistant Enterococcus faecalis also resistant to teicoplanin (VRE Fs Van A), vancomycin resistant Enterococcus faecalis sensitive to teicoplanin (VRE Fs Van B), or penicillin-resistant Streptococcus pneumoniae (PRSP).

In one embodiment of the methods, the bacterial infection is a Bacillusinfection. In a further embodiment, the Bacillus infection is a Bacillus biofilm infection. Bacteria of the genus Bacillus are aerobic, endospore-forming, Gram-positive rods, and infections due to such bacteria are treatable via the methods provided herein. Bacillus species can be found in soil, air, and water where they are involved in a range of chemical transformations. Examples of pathogenic Bacillus species whose infection is treatable with the methods provided herein, include, but are not limited to, B. anthracis, B. cereus and B. coagulans. Several other Bacillus species, e.g., B. subtilis and B. licheniformis, as well as B. cereus, are associated periodically with bacteremia/septicemia, endocarditis, meningitis, and infections of wounds, the ears, eyes, respiratory tract, urinary tract, and gastrointestinal tract, and are therefore treatable with the methods provided herein.

In one embodiment of the methods, a Bacillus anthracis (B. anthracis) infection is treated by the methods disclosed herein. Bacillus anthracis, the infection of which causes anthrax, is acquired via direct contact with infected herbivores or indirectly via their products. The clinical forms of anthrax include cutaneous anthrax, from handling infected material, intestinal anthrax, from eating infected meat, and pulmonary anthrax from inhaling spore-laden dust. The route of administration will vary depending on how the patient acquires the B. anthracis infection. For example, in the case of pulmonary anthrax, the patient, in one embodiment, is treated via a dry powder inhaler, nebulizer or metered dose inhaler.

In one embodiment of the methods, the bacterial infection is a Francisella tularensis (F. tularensis) infection. In a further embodiment, the F. tularensis infection is an F. tularensis biofilm infection. Francisella tularensis is a pathogenic species of Gram-negative, rod-shaped coccobacillus, an aerobe bacterium. It is non-spore forming, non-motile and the causative agent of tularemia, the pneumonic form of which is often lethal without treatment. It is a fastidious, facultative intracellular bacterium which requires cysteine for growth.

In one embodiment of the methods, the bacterial infection is a Burkholderia infection, which is a Gram-negative infection. In a further embodiment, the Burkholderia infection is a Burkholderia biofilm infection. In some embodiments, the Burkholderia infection is a Burkholderia pseudomallei (B. pseudomallei), B. dolosa, B. fungorum, B. gladioli, B. multivorans, B. vietnamiensis, B. ambifaria, B. andropogonis, B. anthina, B. brasilensis, B. calcdonica, B. caribensis, B. caryophylli infection, or a combination of the above infections. Burkholderia is a genus of Proteobacteria whose pathogenic members include among other the Burkholderia cepacia complex which attacks humans; Burkholderia pseudomallei, causative agent of melioidosis; and Burkholderia cepacia, an important pathogen of pulmonary infections in people with cystic fibrosis. The Burkholderia (previously part of Pseudomonas) genus name refers to a group of virtually ubiquitous Gram-negative, obligately aerobic, rod-shaped bacteria that are motile by means of single or multiple polar flagella, with the exception of Burkholderia mallei which is nonmotile.

In one embodiment of the methods, the bacterial infection is a Yersinia pestis (Y. pestis) infection. In a further embodiment, the Y. pestis infection is a Y. pestis biofilm infection. Yersinia pestis (formerly Pasteurella pestis) is a Gram-negative, rod-shaped coccobacillus, non-mobile with no spores. It is a facultative anaerobic organism that can infect humans via the oriental rat flea. It causes the disease plague, which takes three main forms: pneumonic, septicemic, and bubonic plagues.

In one embodiment of the methods, the bacterial infection is a Clostridium infection. In a further embodiment, the Clostridium infection is a Clostridium biofilm infection. Clostridia are spore-forming, Gram-positive anaerobes, and infections due to such bacteria are treatable via the methods provided herein. In one embodiment, the bacterial infection is a Clostridium difficile (C. difficile) infection. In one embodiment, the bacterial infection is a Clostridium tetani (C. tetani) infection, the etiological agent of tetanus. In another embodiment, the bacterial infection is a Clostridium botidinum (C. botidinum) infection, the etiological agent of botulism. In yet another embodiment, the bacterial infection is a C. perfringens infection, one of the etiological agents of gas gangrene. In one embodiment, the bacterial infection is a C. sordellii infection.

In one embodiment of the methods, the bacterial infection is a Corybacterium infection. In a further embodiment, the Corybacterium infection is a Corybacterium biofilm infection. Corynebacteria are small, generally non-motile, Gram-positive, non sporalating, pleomorphic bacilli and infections due to these bacteria are treatable via the methods provided herein. Corybacterium diphtheria is the etiological agent of diphtheria, an upper respiratory disease mainly affecting children, and is treatable via the methods provided herein. Examples of other Corynebacteria species treatable with the methods provided herein include Corynebacterium diphtheria, Corynebacterium pseudotuberculosis, Corynebacterium tenuis, Corynebacterium striatum, and Corynebacterium minutissimum.

In one embodiment of the methods, the bacterial infection is a Nocardia infection. In a further embodiment, the Nocardia infection is a Nocardia biofilm infection. The bacteria of the genus Nocardia are Gram-positive, partially acid-fast rods, which grow slowly in branching chains resembling fungal hyphae. Exemplary Nocardial infections treatable with the methods provided herein include N. aerocolonigenes, N. africana, N. argentinensis, N. asteroides, N. blackwellu, N. brasiliensis, N. brevicalena, N. cornea, N. caviae, N. cerradoensis, N. corallina, N. cyriacigeorgica, N. dassonvillei, N. elegans, N. farcinica, N. nigiitansis, N. nova, N. opaca, N. otitidis-cavarium, N. paucivorans, N. pseudobrasiliensis, N. rubra, N. transvelencesis, N. uniformis, N. vaccinii, and N. veterana infections, and a combination thereof. In one embodiment, the bacterial infection is one selected from the group consisting of an N. asteroides, N. brasiliensis, N. caviae infection, and a combination thereof.

In one embodiment of the methods, the bacterial infection is a Listeria infection. In a further embodiment, the Listeria infection is a Listeria biofilm infection. Listeria are non-spore-forming, nonbranching Gram-positive rods that occur individually or form short chains. Non-limiting examples of Listeria infections treatable with the methods provided herein include L. grayi, L. innocua, L. ivanovii, L. monocytogenes, L. seeligeri, L. murrayi, and L. welshimeri infections, and a combination thereof. In one embodiment, the bacterial infection is a Listeria monocytogenes (L. monocytogenes) infection. In another embodiment, the bacterial infection is an L. monocytogenes infection.

The bacterial infection treatable by the methods provided herein may be present as planktonic free-floating bacteria, a biofilm, or a combination thereof. In one embodiment, the bacterial infection is a planktonic bacterial infection. In another embodiment, the bacterial infection is a bacterial biofilm infection.

In one embodiment of the methods, the bacterial infection is acquired in a healthcare setting, e.g., acquired at a hospital, a nursing home, rehabilitation facility, outpatient clinic, etc. In another embodiment, the bacterial infection is community associated or acquired. In a further embodiment, the bacterial infection is a skin infection. In another embodiment, the bacterial infection is a respiratory tract infection, e.g., pneumonia. In one embodiment, the bacterial infection treated in a pneumonia patient is an S. pneumoniae infection. In another embodiment, the bacterial infection treated in a pneumonia patient is Mycoplasma pneumonia or a Legionella species. In another embodiment, the bacterial infection in a pneumonia patient is penicillin resistant, e.g., penicillin-resistant S. pneumoniae. In another embodiment, the pneumonia is due to S. aureus, e.g., MRSA.

Respiratory bacterial infections and in particular pulmonary bacterial infections are quite problematic for cystic fibrosis (CF) patients. In fact, such infections are the main cause of pulmonary deterioration in this population of patients. The lungs of CF patients are colonized and infected by bacteria from an early age. These bacteria thrive in the altered mucus, which collects in the small airways of the lungs. The formation of biofilms makes infections of this origin difficult to treat. Consequently, more robust treatments options are needed. Thus, in one embodiment, the methods disclosed herein are useful in treating a patient with cystic fibrosis having a bacterial infection. In a further embodiment, the bacterial infection is a pulmonary bacterial infection. In a further embodiment, the pulmonary bacterial infection is a pulmonary MRSA infection. In a further embodiment, the pulmonary infection is comprised of a biofilm. In a further embodiment, the pharmaceutical composition administered according to the disclosed methods comprises RV94, RV40, or oritavancin.

The methods disclosed herein are also useful in treating a patient with osteomyelitis having a bacterial infection of the bone. In a further embodiment, the patient with osteomyelitis has a Staphylococcus aureus infection of the bone. In a further embodiment, the Staphylococcus aureus infection is an MRSA infection. In a further embodiment, the

MRSA infection is an MRSA biofilm infection. In a further embodiment, the pharmaceutical composition administered according to the disclosed methods comprises RV94, RV40, or oritavancin.

### EXAMPLES

The present invention is further illustrated by reference to the following Examples. However, it should be noted that these Examples, like the embodiments described above, are illustrative and are not to be construed as restricting the scope of the invention in any way.

### Example 1—Development of Alkylamine Modified poly(L-glutamic acid) Polymer-Based Tunable Drug Delivery Platform

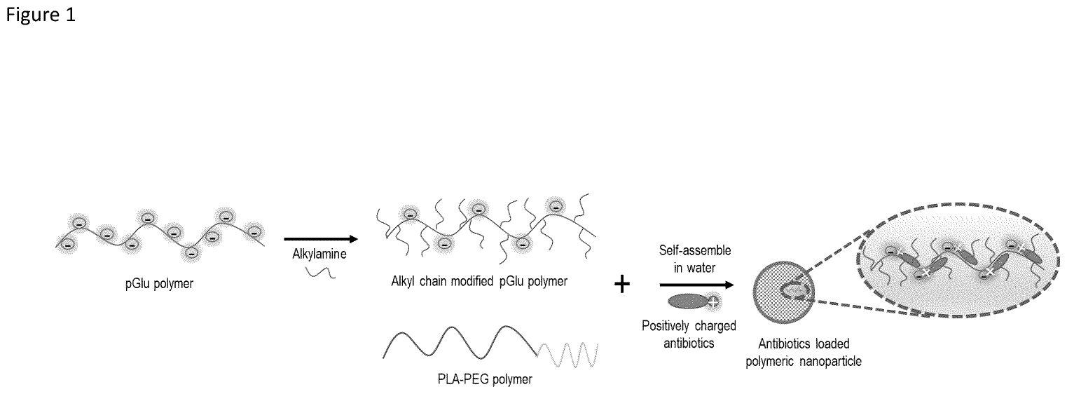

Targeted delivery of antibiotics with polymeric nanoparticles (PNPs) may provide benefits such as a high local drug concentration and sustained drug release at the site of infection and decreased systemic toxicities. We focused our efforts on targeted delivery of vancomycin lipophilic derivatives. Since lipoglycopeptides are typically positively charged at neutral pH and slightly soluble in water, they may not be encapsulated efficiently into nanoparticles with core materials that are hydrophobic and charge neutral polymers, such as polylactic acid (PLA). To overcome this challenge, we describe in this example the development of alkylamine modified poly(L-glutamic acid) (pGlu) polymers with a tunable negative charge/hydrophobicity ratio as a delivery platform for vancomycin and its lipophilic derivatives. In those modified polymers, the carboxylic acid groups are replaced by alkylamines in varying degrees. As a result, the negative charge density-to-hydrophobicity ratio of the modified polymers can be tuned to efficiently load and encapsulate vancomycin and its derivative lipoglycopeptides in the presence of PLA-PEG (FIG. 1).

### 1. Synthesis and Characterization of pGlu(0.6C₁₀), an Alkylamine Modified pGlu with Approximately 30% of the Carboxylic Acid Groups of pGlu Replaced by Decylamine (C10A)

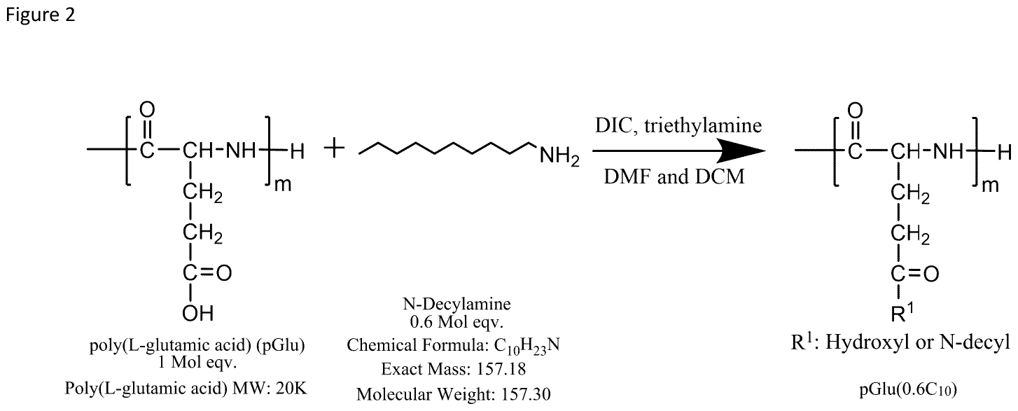

pGlu(0.6C₁₀) was synthesized by a coupling reaction between poly-L-glutamic acid (20K) and N-decylamine (abbreviated as C10A) with a COOH:C10A ratio at 1:0.6 in the presence of the coupling agent N,N′-diisopropylcarbodiimide (DIC) (FIG. 2). Specifically, a 20 ml vial was charged with 267 mg pGlu and 5 ml dimethylformamide (DMF). The mixture was stirred at 380 rpm for 3 min at 80° C., followed by cooling to room temperature after all of the polymer was dissolved. Then 2 ml dichloromethane (DCM) was added, followed by the addition of 285 mg DIC and 193.6 mg N-decylamine. After overnight stirring, 40.75 mg DIC and 36.3 mg triethylamine were added in the solution and the reaction mixture was let sit for 0.5 h. The mixture was then separated into two 50 ml falcon tubes (polypropylene) and 35 ml tert-butyl methyl ether was introduced as the antisolvent to each tube. The dissolved polymer was precipitated by adding 200 μl ammonium hydroxide. The crude product was centrifuged for 10 min at the maximum speed at −9° C. The supernatant was decanted, and the polymer in the two tubes was dissolved by adding 1 ml of acetonitrile (ACN), 1 ml of acetone and 1 ml of 1-propanol to each tube, followed by vortexing and sonication. After the dissolved polymer was combined, it was subjected to further purification by adding 35 ml tert-butyl methyl ether followed by centrifugation. This step was performed to remove any unreacted amine and DIC from the insoluble material. The polymer was dissolved in 1 ml of ACN, 1 ml of acetone, 1 ml of 1-propanol and 1 ml of deionized water. After vortexing and sonication, the polymer was decanted into 50 ml 1:50 acetic acid (v/v) in water solution while stirring. This step was performed to remove any unreacted amine and DIC from the insoluble material. Using funnel filtration, the purified polymer was collected in a funnel, followed by lyophilization to dryness in a lyophilizer to afford pGlu(0.6C₁₀) as off-white crystal solids, with an overall yield of 65%.

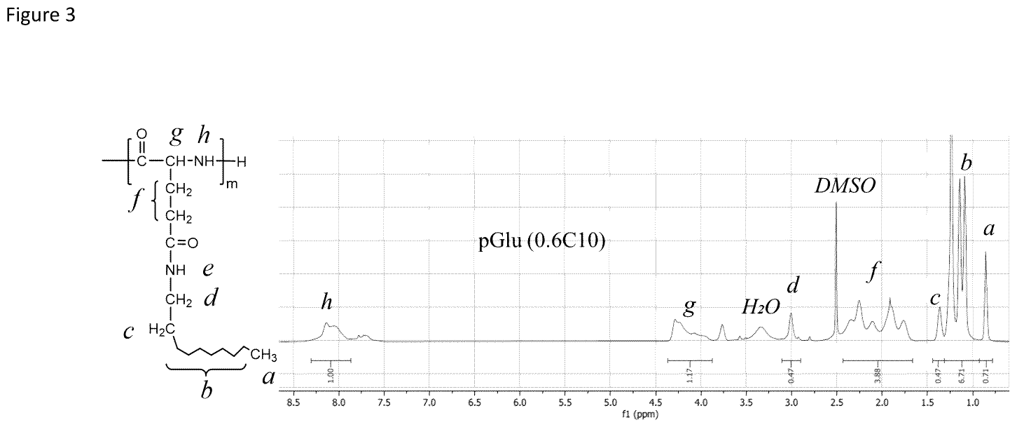

In the synthesized pGlu(0.6C₁₀), about 30% of the carboxylic acid groups of pGlu were replaced by C10A, as determined by ¹H NMR. FIG. 3 shows the NMR data in which we were able to identify and assign each of the peaks. Protons on peaks of f, g and h are from the backbone of the product polymer pGlu(0.6C₁₀); protons on peaks of a, b, c, d and e are from the alkyl chain of the product polymer pGlu(0.6C₁₀). To calculate the replacement rate of glutamic acid by C10A, the integral value of the methyl protons (a) on the alkyl chain of pGlu(0.6C₁₀) was compared with that of the secondary amine protons (h) on the backbone of pGlu(0.6C₁₀). The data also confirms that pGlu(0.6C10) had been successfully synthesized.

### 2. Fabrication and Characterization of Drug Loaded PNPs Comprising pGlu(0.6C₁₀)

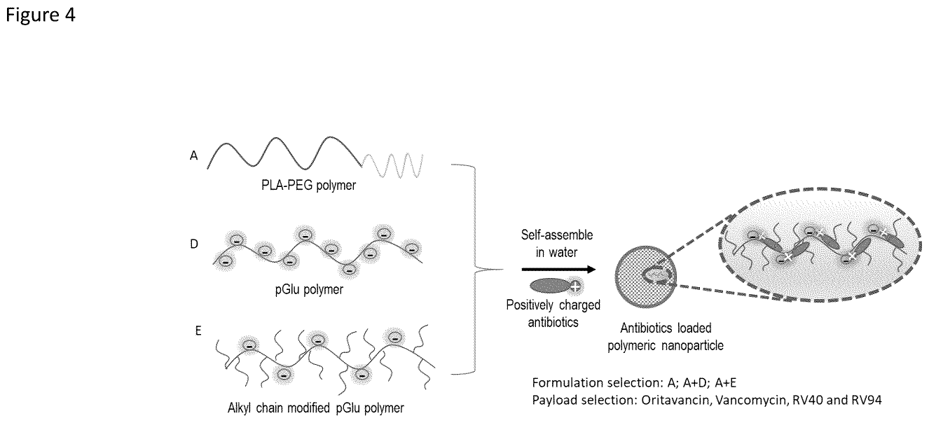

To assess the ability of pGlu(0.6C₁₀) to load positively charged antibiotics in the form of PNPs, we selected vancomycin, oritavancin, RV40 and RV94 as the drug payload. Vancomycin was chosen as a non-lipoglycopeptide control and the other three payloads were lipoglycopeptides. The polymers we used for loading and encapsulating the antibiotics were PLA(10K)-PEG(2K) (A) obtained from Nanosoft Polymers (Winston-Salem, NC, USA; SKU: 2693), pGlu (D) also obtained from Nanosoft Polymers (SKU: 10069), and pGlu(0.6C₁₀) (E) (FIG. 4). To demonstrate that both the negative charge and hydrophobicity from the alkyl chain of pGlu(0.6C₁₀) contributed to efficient loading and encapsulation of the drugs, we prepared drug loaded nanoparticles containing A/drug, (A+D)/drug and (A+E)/drug and compared their encapsulation efficiencies (EE) (Table 2).

TABLE 2
Comparison of drug loading and encapsulation efficiencies of various drug loaded PNPs

| | | | | | | Measured by HPLC | | | | |
|---|---|---|---|---|---|---|---|---|---|---|
| | | Polymer | | | | Drug | | Drug | Result | |
| | | PLA- | | | | (mg) | Drug | (mg) | | Encapsulation |
| | | PEG | pGlu | pGlu(0.6C₁₀) | Drug | before | (mg) | after | Drug | efficiency |
| | | (mg) | (mg) | (mg) | (mg) | wash | filtrate | wash | loading | (EE) |
| A | Ori | 10 | N/A | N/A | 2.5 Ori | 2.38 | 1.98 | 0.23 | 2.3% | 9.8% |
| A + D | | 5 | 5 | N/A | 2.5 Ori | 2.43 | 1.88 | 0.96 | 8.7% | 39.3% |
| A + E | | 5 | N/A | 5 | 2.5 Ori | 2.31 | 0.34 | 1.40 | 12.3% | 60.6% |
| N/A | | N/A | N/A | N/A | 2.5 Ori | 2.16 | 2.43 | 0.00 | 0.0% | 0.0% |
| A | Vac | 10 | N/A | N/A | 2.5 Vac | 1.01 | 1.00 | 0.00 | 0.0% | 0.2% |
| A + D | | 5 | 5 | N/A | 2.5 Vac | 1.16 | 0.77 | 0.00 | 0.0% | 0.1% |
| A + E | | 5 | N/A | 5 | 2.5 Vac | 1.08 | 0.75 | 0.01 | 0.1% | 0.5% |
| N/A | | N/A | N/A | N/A | 2.5 Vac | 1.10 | 0.87 | 0.00 | 0.0% | 0.0% |
| A | RV40 | 10 | N/A | N/A | 2.5 RV40 | 2.72 | 2.79 | 0.19 | 1.9% | 7.0% |
| A + D | | 5 | 5 | N/A | 2.5 RV40 | 2.15 | 2.00 | 0.32 | 3.1% | 15.0% |
| A + E | | 5 | N/A | 5 | 2.5 RV40 | 1.75 | 0.33 | 1.76 | 15.0% | 100.7% |
| N/A | | N/A | N/A | N/A | 2.5 RV40 | 2.49 | 3.19 | 0.00 | 0.0% | 0.0% |
| A | RV94 | 10 | N/A | N/A | 2.5 RV94 | 2.81 | 2.38 | 0.29 | 2.8% | 10.4% |
| A + D | | 5 | 5 | N/A | 2.5 RV94 | 2.84 | 2.26 | 0.37 | 3.5% | 12.9% |
| A + E | | 5 | N/A | 5 | 2.5 RV94 | 2.47 | 0.73 | 2.65 | 20.9% | 107.2% |
| N/A | | N/A | N/A | N/A | 2.5 RV94 | 3.06 | 2.22 | 0.13 | 1.3% | 4.2% |

We prepared the drug loaded PNPs by using 10 mg of polymers and 2.5 mg of drug (FIG. 4). Specifically, polymers (10 mg) and drug (2.5 mg) were dissolved in 1.2 ml organic solvent mixture (DMSO: acetonitrile=0.5:0.7). The solution was injected into 10 ml deionized water stirred with a magnetic stirring bar. 1.1 ml 10×PBS solution was added to suspend nanoparticles in 1×PBS solution. The nanoparticles were washed using Amicon filter (100k cut-off) twice and resuspended in PBS. Drug loading in nanoparticle formulation was determined by HPLC with a WATERS™ T3 column. Acetonitrile/water was used as the mobile phase, and the wavelength of the UV detector was set at 281 nm.

$$Drug loading is equal to mass of drug ( mg ) after wash / ( mass of drug ( mg ) after wash + mass of polymer input ( i . e . , 10 mg ) ) ⋆ 100 \% .$$

EE is equal to mass of drug (mg) after wash/mass of drug input (i.e., 2.5 mg) *100%. With EE calculated as such, an EE of over 100% may be obtained on certain occasions due to standard error within the analytical measurement and represents complete encapsulation (i.e., 100% EE).

The size of the nanoparticles was measured by dynamic light scattering (Mobius, Wyatt Technology).

As shown in Table 2, PNPs containing PLA-PEG (A) as the only polymer and loaded with vancomycin, oritavancin, RV40, or RV94 as the drug displayed EE of 0.2%, 9.8%, 7.0% and 10.4% respectively.

Also as shown in Table 2, PNPs containing a polymer combination of PLAPEG (A) and pGlu (D) and loaded with vancomycin, oritavancin, RV40, or RV94 showed EE of 0.1%, 39.3%, 15.0% and 12.9% respectively. The increase in lipoglycopeptide loading as compared to that using the PLA-PEG (A)-only nanoparticles indicated that negative charge contributes to the improved encapsulation efficiency.

Further, the encapsulation efficiency of vancomycin, oritavancin, RV40 and RV94 using PNPs containing a polymer combination of PLA-PEG (A) and pGlu(0.6C₁₀) (E) reached 0.5%, 60.6%, 100.7%, and 107.2%, respectively, which were higher than the corresponding encapsulation efficiencies using the PNPs containing PLA-PEG (A) as the only polymer, and the corresponding encapsulation efficiencies using the PNPs containing a polymer combination of PLA-PEG (A) and pGlu (D). These data indicate that both the negative charge and hydrophobicity from the alkyl modification in pGlu(0.6C₁₀) (E) promote encapsulation of the drugs. Formulations containing only the drug payload with no polymer (N/A) exhibited no or minimal drug encapsulation and no nanoparticle formation.

### Example 2—Preparation of Stable and High Drug Loading RV40-PNP Formulation Using a Scalable Continuous Flow Nanoprecipitation Manufacture Process

pGlu(0.6C₁₀)-containing PNP formulations with even greater drug loading were explored by adjusting the ratio of each component. RV40 was selected as the drug payload. The polymers used for RV40 encapsulation were PLA(10K)-PEG(2K) (Nanosoft Polymers; SKU: 2693), PLA(10K)-PEG(5K)-biotin (Nanosoft Polymers, SKU: 24000), PLA(16K)-Cy5, and pGlu(0.6C₁₀). PLA(10K)-PEG(5K)-biotin was a derivative of PLA(10K)-PEG(5K) where biotin was covalently attached to the end of the PEG block of PLA(10K)-PEG(5K).

PLA(16K)-Cy5 was a derivative of PLA(16K) where the fluorescent label Cy5 was grafted at the branch of PLA(16K). PLA(16K)-Cy5 was synthesized by a coupling reaction between PLA-COOH(16K) (Durect) and Cyanine5 amine (abbreviated as Cy5 amine) (Lumiprobe, CAS number: 1807529-70-9) with a COOH:Cy5 ratio at 1:0.2 in the presence of the coupling agent N,N′-diisopropylcarbodiimide (DIC) and the Hünig's base N,N-Diisopropylethylamin (DIEA). Specifically, a 20 ml vial was filled with 4 ml dichloromethane (DCM) and 1 ml dimethylformamide (DMF). 3 g PLA-COOH and 25 mg Cy5 amine were dissolved in DCM/DMF. 15 ul DIEA was added to neutralize the HCl, followed by adding 15 ul DIC. The mixture was stirred overnight at room temperature. The product was precipitated by pouring the reaction mixture into cold diethyl ether and collecting the precipitate by centrifugation. The product was redissolved in DCM and precipitated again with cold diethyl ether. Then the product was dried under vacuum for one day. In this step, about 20% of the carboxylic acid groups of PLA-COOH were replaced by Cy5. To block the free COOH of PLA, a similar procedure was performed with ethanolamine. Specifically, a 20 ml vial was filled with 4 ml dichloromethane (DCM) and 1.5 ml dimethylformamide (DMF). 3 g PLA-Cy5 was dissolved in DCM/DMF. 28.3 ul (COOH: amine ratio at 1:2.5) ethanolamine (Sigma) and 75 ul DIC were added. The mixture was stirred overnight at room temperature. The product was precipitated by pouring the reaction mixture into cold diethyl ether and collecting the precipitate by centrifugation. The product was redissolved in DCM and precipitated again with cold diethyl ether. Then the final product of PLA(16K)-Cy5 was dried under vacuum for one day, with an overall yield of 90%.

pGlu(0.6C₁₀) was prepared as described in Example 1, with the replacement rate of glutamic acid by C10A at about 22%. The mass and mole ratio for each component are shown in Table 3. Ammonium hydroxide was selected as the base and the mole ratio of glutamic unit: modified glutamide unit: RV40: base was 0.78:0.22:0.11:0.67, which facilitated a pH neutral PNP suspension.

TABLE 3
Components in RV40-PNP preparation

| | PLA- | PLA-PEG- | | | RV40 | |
|---|---|---|---|---|---|---|
| Component | PEG | Biotin | pGlu(0.6C10) | PLA-Cy5 | acetate | NH₄OH |
| Mass ratio | 10 | 1 | 42 | 2 | 50 | N/A |
| Mole ratio | N/A | N/A | 0.78 | N/A | 0.11 | 0.67 |
| to Glu unit | | | | | | |

The drug loaded PNPs were prepared with 2.475 g of polymers and 2.25 g RV40 using a scalable continuous flow nanoprecipitation manufacture process. Specifically, the excipients and API (i.e., RV40) were dissolved in a 1:1 DMSO: ACN. The PNP suspension was produced by infusing 6% organic phase into aqueous phase through continuous flow nanoprecipitation process. Tangential flow filtration (TFF) system was used to purify and concentrate PNPs. The nanoparticle size was determined by dynamic light scattering. The drug concentration was determined by HPLC. The formulation concentration was determined by mass measurement after removing suspension media by evaporation using a SpeedVac. The drug loading was determined to be 54.1% (Table 4), which was markedly higher than that seen in the study of Example 1. The RV40-PNP formulation demonstrated physical (size) and drug retention stability. After freezing at −20° C. and thawing and a second wash, the size and drug concentration were nominally the same as in the released batch (Table 4).

TABLE 4
RV40-PNP characterization

| | | | | | Size stability (nm) (PdI) | | | |
|---|---|---|---|---|---|---|---|---|
| | | | | | Drug | | | |
| | | | | | concentration | | | |
| Drug retention | | | | | after second | | | |
| Drug | PNP | | | | wash in | After | | After |
| Concentration | Concentration | Drug/ | Drug | | freeze & thaw | first | Freeze | second |
| (mg/ml) | (mg/ml) | polymer | loading | EE | (mg/ml) | wash | & thaw | wash |
| 5.64 | 10.43 | 1.18 | 54.10% | 37.40% | 5.46 | 73 | 63.5 | 51.4 |
| | | | | | | (0.264) | (0.333) | (0.281) |

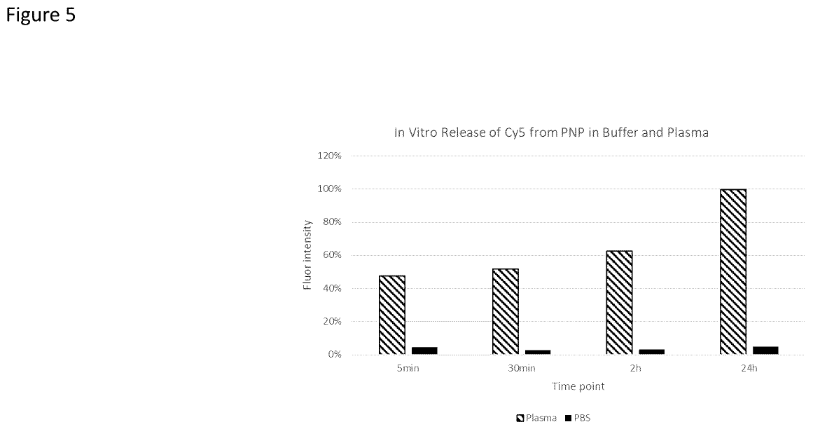

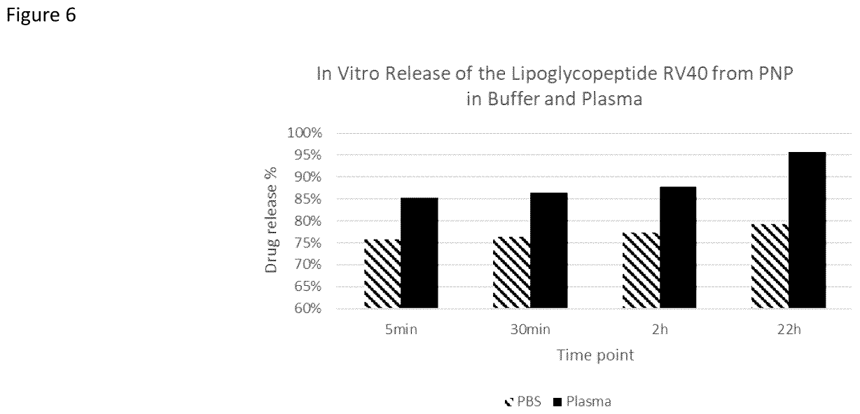

To determine RV40-PNP in vitro release kinetics, a customized magnetic avidin beads method was applied, where magnetic beads decorated with streptavidin rapidly and strongly bound biotin modified PNPs through an interaction between biotin on PLA(10K)-PEG(5K)-biotin and streptavidin. After applying a magnet, nanoparticles were efficiently separated from supernatant. The supernatant was collected at various time points and the in vitro release kinetics determined. Additionally, since an inert hydrophobic fluorophore was loaded inside of nanoparticles, the separation efficiency and PNP stability was orthogonally tracked by measurement of fluorescent intensity in the release medium. FIGS. 5 and 6 show the RV40 PNP stability and nanoparticle release kinetics in PBS and plasma. As shown in FIG. 5, in PBS, binding of the beads to PNPs efficiently separated the nanoparticles from the supernatant with no obvious leakage of fluorophore. By contrast, PNPs incubated in plasma were less stable as indicated by greater hydrophobic fluorophore leak in the supernatant, presumably due to the plasma protein binding on the PNP surface. As shown in FIG. 6, the RV40-PNP formulation showed a burst release of RV40 up to 75-85% in 5 min, followed by a slow release in both PBS and the plasma.

### Example 3—Development of Alkylamine Modified poly(L-glutamic acid)-block-poly(ethylene glycol) Polymer-Based Tunable Drug Delivery Platform

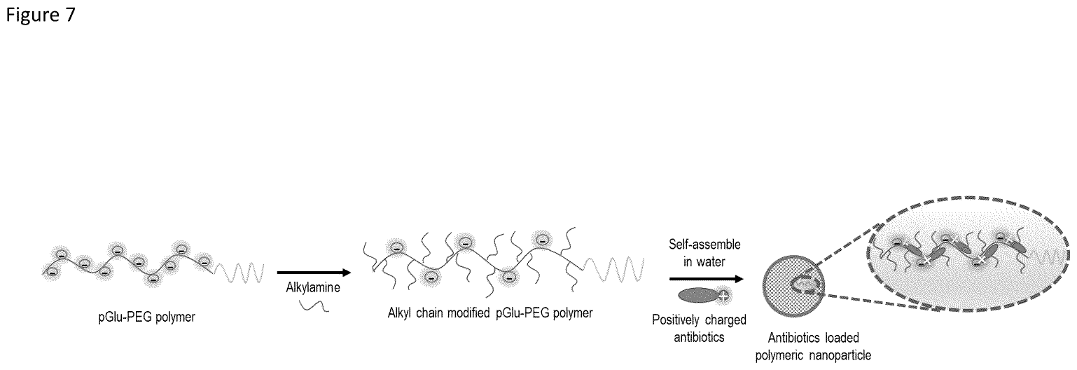

In this example, we describe the development of alkylamine modified poly(L-glutamic acid)-block-poly(ethylene glycol) polymers with a tunable negative charge-to-hydrophobicity ratio as another delivery platform for vancomycin and its lipophilic derivatives. The modified polymers are similar to those described in Example 1 in that the carboxylic acid groups can be replaced by alkylamines in varying degrees. As a result, the negative charge density-to-hydrophobicity ratio of the modified polymers likewise can be tuned to efficiently load and encapsulate vancomycin and its derivative lipoglycopeptides (FIG. 7).

### 1. Synthesis and Characterization of pGlu(0.6C₁₀)-PEG, an Alkylamine Modified poly(L-glutamic acid)-block-poly(ethylene glycol) Polymer with about 36% of the Carboxylic Acid Groups of pGlu replaced by Decyl Amine (C10A)

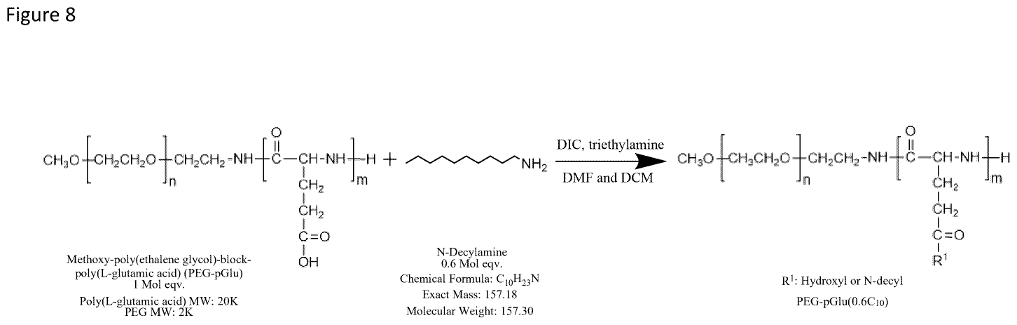

pGlu(0.6C₁₀)-PEG was synthesized by a coupling reaction between pGlu(20K)-PEG(2K) and decyl amine (abbreviated as C10A) with a COOH:C10A ratio at 1:0.6 in the presence of the coupling agent N,N′-diisopropylcarbodiimide (DIC) (FIG. 8). Specifically, a 20 ml vial was charged with 267 mg methoxy-poly(ethylene glycol)-block-poly(L-glutamic acid) (MPEG-pGlu) and 5 ml DMF. The mixture was stirred at 380 rpm for 3 min at room temperature. After all the polymer is dissolved, 2 ml DCM was added, followed by the addition of 285 mg DIC and 176 mg N-decylamine. After overnight stirring, 40.75 mg DIC and 36.3 mg triethylamine were added in the solution and the reaction mixture was let sit for 30 min. The mixture was then separated into two 50 ml falcon tubes (polypropylene) and 35 ml tert-butyl methyl ether was introduced as the antisolvent to each tube. The dissolved polymer was precipitated by adding 200 μl ammonium hydroxide. The crude product was centrifuged for 10 min at the maximum speed at −9° C. The supernatant was decanted, and the polymer in the two tubes was dissolved by adding 1 ml of ACN, 1 ml of acetone and 1 ml of 1-propanol to each tube, followed by vortexing and sonication. After the dissolved polymer was combined, it was subjected to further purification by adding 35 ml tert-butyl methyl ether followed by centrifugation. This step was performed to remove any unreacted amine and DIC from the insoluble material. The polymer was dissolved in 1 ml of ACN, 1 ml of acetone, 1 ml of 1-propanol and 1 ml of deionized water. After vortexing and sonication, polymer was decanted into 50 ml 1:50 acetic acid (v/v) in water solution while stirring. This step was performed to remove any unreacted amine and DIC from the insoluble material. Using funnel filtration, the purified polymer was collected in a funnel, followed by lyophilization to dryness in a lyophilizer to afford pGlu (0.6C₁₀)-PEG as off-white crystal solids with an overall yield of 60%.

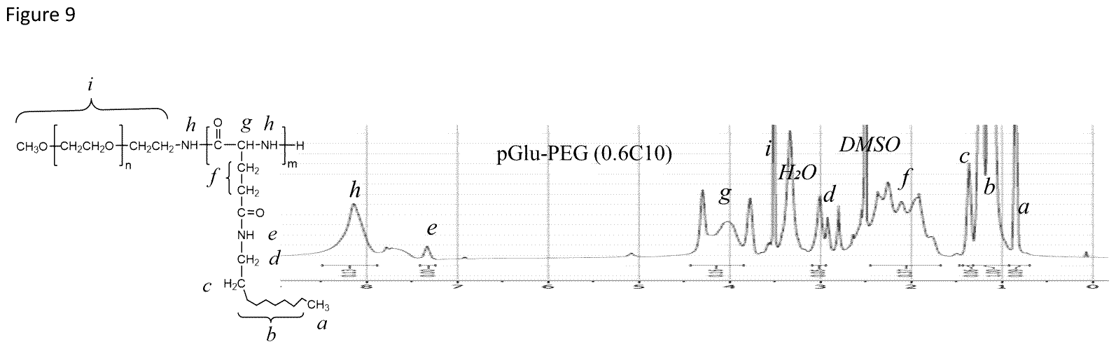

In the synthesized pGlu (0.6C₁₀)-PEG, approximately 36% of the carboxylic acid groups of pGlu were replaced by C10A, as determined by ¹H NMR. FIG. 9 shows the NMR data in which we were able to identify and assign each of the peaks. Protons on peaks of f, g and h are from the backbone of the product polymer pGlu (0.6C₁₀)-PEG; protons on peaks of a, b, c, d and e are from the alkyl chain of the product polymer pGlu (0.6C₁₀)-PEG. To calculate the replacement rate of glutamic acid by C10A, the integral value of the methyl protons (a) on the alkyl chain of pGlu(0.6C₁₀)-PEG was compared with that of the secondary amine protons (h) on the backbone of pGlu(0.6C₁₀)-PEG. The data also confirms that pGlu(0.6C₁₀)-PEG had been successfully synthesized.

### 2. Fabrication and Characterization of Drug Loaded PNPs Comprising pGlu(0.6C₁₀)-PEG

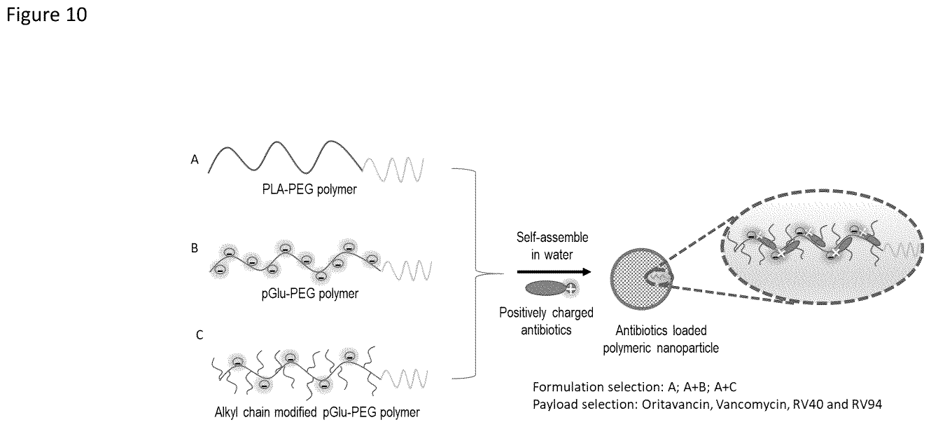

To assess the ability of pGlu(0.6C₁₀)-PEG to load positively charged antibiotics in the form of PNPs, we selected vancomycin and RV94 as the antibiotic drug payload in a first study (study 1) (Table 5), and additionally included oritavancin and RV40 as the antibiotic drug payload in a second study (study 2) (Table 6). The polymers we used for loading and encapsulating the antibiotics were PLA-PEG (A) (Nanosoft Polymers, SKU: 2693), pGlu-PEG (B) (Nanosoft Polymers, SKU: 2917), and pGlu(0.6C₁₀)-PEG (C) (FIG. 10). To demonstrate that both the negative charge and hydrophobicity from the alkyl chain of pGlu(0.6C₁₀)-PEG contributed to efficient encapsulation, we prepared drug loaded PNPs containing A/drug, (A+B)/drug and (A+C)/drug and compared their encapsulation efficiencies (EE) (Tables 5 and 6).

TABLE 5
PNP loading and encapsulation of vancomycin and RV94 in study 1

| | | | | | | Measured | | | |
|---|---|---|---|---|---|---|---|---|---|
| | | Formulation | | | | by HPLC | | | |
| | | Polymer | | | | Drug | Drug | Result | |
| | | PLA- | pGlu- | pGlu(0.6C₁₀)- | | (mg) | (mg) | | Encapsulation |
| | | PEG | PEG | PEG | Drug | before | after | Drug | efficiency |
| | | (mg) | (mg) | (mg) | (mg) | wash | wash | loading | (EE) |
| A | Vac | 20 | N/A | N/A | 5 Vac | 3.86 | 0.00 | 0.0% | 0.0% |
| A + B | | 10 | 10 | N/A | 5 Vac | 4.94 | 0.00 | 0.0% | 0.0% |
| A + C | | 10 | N/A | 10 | 5 Vac | 2.94 | 0.55 | 2.7% | 18.6% |
| A | RV94 | 20 | N/A | N/A | 5 RV94 | 4.50 | 0.18 | 0.9% | 4.0% |
| A + B | | 10 | 10 | N/A | 5 RV94 | 4.50 | 3.15 | 13.6% | 70.1% |
| A + C | | 10 | N/A | 10 | 5 RV94 | 4.54 | 4.01 | 16.7% | 88.4% |

TABLE 6
PNP loading and encapsulation of vancomycin, oritavancin, RV40 and RV94 in study 2

| | | | | | | Measured | | | |
|---|---|---|---|---|---|---|---|---|---|
| | | Formulation | | | | by HPLC | | | |
| | | Polymer | | | | Drug | Drug | Result | |
| | | PLA- | pGlu- | pGlu(0.6C₁₀)- | | (mg) | (mg) | | Encapsulation |
| | | PEG | PEG | PEG | Drug | before | after | Drug | efficiency |
| | | (mg) | (mg) | (mg) | (mg) | wash | wash | loading | (EE) |
| A + B | Ori | 10 | 10 | N/A | 5 Ori | 5.8 | 2.4 | 10.6% | 41.0% |
| A + C | | 10 | N/A | 10 | 5 Ori | 4.6 | 3.1 | 13.4% | 68.1% |
| A + B | Vac | 10 | 10 | N/A | 5 Vac | 5.0 | 0.008 | 0.0% | 0.2% |
| A + C | | 10 | N/A | 10 | 5 Vac | 5.0 | 0.5 | 2.4% | 9.8% |
| A + B | RV40 | 10 | 10 | N/A | 5 RV40 | 4.3 | 1.4 | 6.7% | 33.2% |
| A + C | | 10 | N/A | 10 | 5 RV40 | 3.0 | 1.4 | 6.4% | 45.5% |
| A + B | RV94 | 10 | 10 | N/A | 5 RV94 | 5.1 | 3.0 | 13.2% | 59.8% |
| A + C | | 10 | N/A | 10 | 5 RV94 | 4.6 | 4.3 | 17.5% | 92.7% |

We prepared the drug loaded PNPs by using 20 mg of polymers and 5 mg of drug (FIG. 10). Specifically, polymers (20 mg) and drug (5 mg) were dissolve in 1 ml organic solvent mixture (DMSO: acetonitrile=1:1). The solution was injected into 20 ml deionized water stirred with a magnetic stirring bar. The nanoparticles were washed using Amicon filter (100k cut-off) twice and resuspended in deionized water. Drug loading in nanoparticle formulation was determined by HPLC with a WATERS™ T3 column. Acetonitrile/water was used as the mobile phase, and the wavelength of the UV detector was set at 281 nm.

Drug loading is equal to mass of drug (mg) after wash/(mass of drug (mg) after wash+mass of polymer input (i.e., 20 mg))*100%.

EE is equal to mass of drug (mg) after wash/mass of drug input (i.e., 5 mg)*100%.

The size of the nanoparticles was measured by dynamic light scattering (Mobius, Wyatt Technology).

As shown in Table 5, PNPs containing PLA-PEG (A) as the only polymer and loaded with vancomycin or RV94 showed an EE of 0.0% and 4.0%, respectively. By comparison, PNPs containing a polymer combination of PLA-PEG (A) and pGlu-PEG (B) and loaded with vancomycin or RV94 showed an EE of 0.0% and 70.1%, respectively. The increase in RV94 encapsulation indicates that the negative charge of pGlu-PEG (B) contributes to the improved encapsulation efficiency. That the polymer combination of PLA-PEG (A) and pGlu-PEG (B) resulted in no improvement over PLA-PEG (A) alone for the encapsulation of vancomycin, which is more hydrophilic than RV94, indicates that negative charge alone is not sufficient. As a further comparison, PNPs containing a polymer combination of PLA-PEG (A) and pGlu(0.6C₁₀)-PEG (C) exhibited even better encapsulation efficiencies for both vancomycin and RV94 as compared to PNPs containing PLA-PEG (A) as the only polymer and PNPs containing a polymer combination of PLA-PEG (A) and pGlu-PEG (B), indicating that both the negative charge and hydrophobicity from the alkyl modification in pGlu(0.6C₁₀)-PEG promote encapsulation of the drugs.

Similar results were obtained in study 2, in which PNP formulations containing either a polymer combination of PLA-PEG (A) and pGlu-PEG (B), or a polymer combination of PLA-PEG (A) and pGlu(0.6C₁₀)-PEG (C), and one antibiotic drug payload selected from vancomycin, oritavancin, RV40, or RV94 were compared (Table 6). The PNP formulations containing a polymer combination of PLA-PEG (A) and pGlu(0.6C₁₀)-PEG (C) consistently exhibited an increased EE for each of the antibiotics as compared to the PNP formulations containing a polymer combination of PLA-PEG (A) and pGlu-PEG (B). These data confirm that both the negative charge and hydrophobicity from the alkyl modification in pGlu(0.6C₁₀)-PEG contribute to enhanced encapsulation of the drugs.

### Example 4—Pharmacokinetic (PK) Study of Oritavancin-Loaded PNPs in a Rat Osteomyelitis Model

In this example, pharmacokinetic profiles of oritavancin-loaded PNPs containing a polymer combination of PLA(10K)-PEG(2K) and pGlu(0.6C₁₀)-PEG, oritavancin-loaded PNPs containing PLA(10K)-PEG(2K) as the only polymer, and free oritavancin in a rat osteomyelitis model were determined and compared.

### Methods

### 1. Preparation of Oritavancin-Loaded PNPs

Oritavancin-loaded PNPs containing a polymer combination of PLA(10K)-PEG(2K) and pGlu(0.6C₁₀)-PEG (ORI-hybrid PNP) and oritavancin-loaded PNPs containing PLA(10K)-PEG(2K) as the only polymer (ORI-PLA PNP) were prepared as described in Example 3. The compositions of ORI-hybrid PNP and ORI-PLA PNP, as well as the composition of free oritavancin (ORI), are detailed in Table 7 below.

TABLE 7
Compositions of ORI-hybrid PNP, ORI-PLA PNP, and ORI

| | Composition | | | Oritavancin | |
|---|---|---|---|---|---|
| | PLA(10K)- | pGlu(0.6C₁₀)- | | Concentration | PNP Size |
| | PEG(2K) | PEG | Oritavancin | (mg/ml) | (nm) |
| ORI-hybrid PNP | 33.3% | 33.3% | 33.3% | 3 | 127.8 |
| ORI-PLA PNP | 60.0% | N/A | 40.0% | 3 | 141.9 |
| ORI | N/A | N/A | 100% | 3 | N/A |

### 2. Generation of Rat Model of Bacterial Osteomyelitis of the Tibia

A rat model of bacterial osteomyelitis of the tibia was generated via intraosseous injection of MRSA ATCC 43300 at a target challenge dose of 7 log10 CFU of the bacteria per rat. Briefly, rats were anaesthetized by isoflurane and the infection was initiated via injection of the bacteria through the upper condyle of the right tibia into the cancellous bone marrow of a rat. The inoculation was made on the anterior surface of the tibia. The area was located by running a ½ inch 27-gauge needle against the skin, along the anterior crest of the tibia. The needle slipped over the tip of the tuberosity, which was located between the medial and lateral condyles. A rough depression could be felt with the tip of the needle. The patella tendon (silver in color) connected at this point. Once identified, the needle was pushed into the metaphysis using slight pressure and rotating motion. The needle was removed and replaced with a new ½ inch 27 gauge needle, and 30 μL of bacteria was inoculated into the bone marrow cavity. The needle was removed and the animal was allowed to recover from anesthesia.

### 3. Administration of Oritavancin-Loaded PNPs or Free Oritavancin and Collection of Plasma and Tibia Tissues

Three weeks (21 days) after the initiation of the bacterial infection, the osteomyelitis rats were divided into three treatment groups, with five rats in each group. Group 1 rats were administered via intravenous injection 6 mg/kg of ORI-hybrid PNP. Group 2 rats were administered via intravenous injection 6 mg/kg of ORI-PLA PNP. Group 3 rats were administered via intravenous injection 6 mg/kg of ORI. Plasma was collected 30 min, 2 h, 4 h, 6 h, and 24 h post-intravenous injection. The rats were sacrificed 24 h post-intravenous injection and both the uninfected and the infected tibias of each rat were collected and homogenized. The concentrations of oritavancin in the plasma and tibia samples were quantified using LC-MS/MS.

### Results

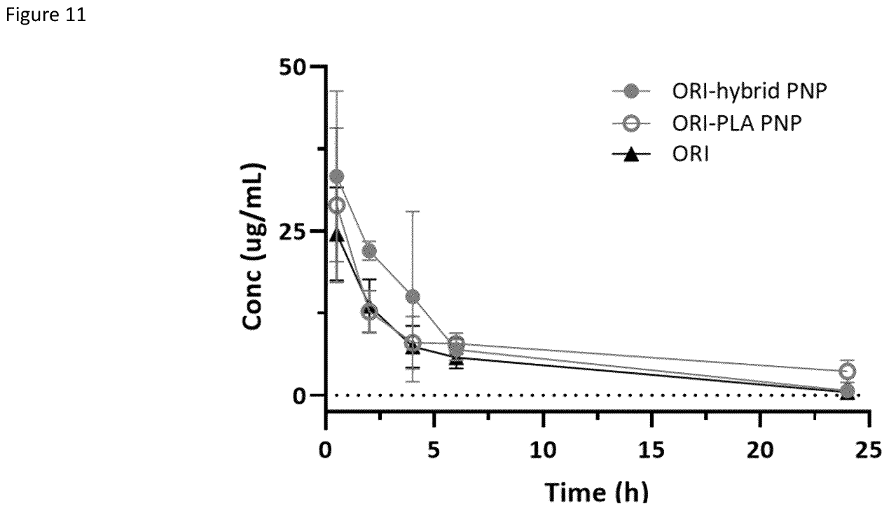

FIG. 11 shows the plasma PK results from the three treatment groups of rats. Group 1 rats receiving ORI-hybrid PNP exhibited the highest plasma concentrations of oritavancin up to 4 hours post-intravenous injection of ORI-hybrid PNP, whereas Groups 2 and 3 rats receiving ORI-PLA PNP and ORI, respectively, displayed comparable and lower plasma concentrations of oritavancin during the same time period, indicating that ORI-hybrid PNP is better than ORI-PLA PNP in drug retention in vivo.

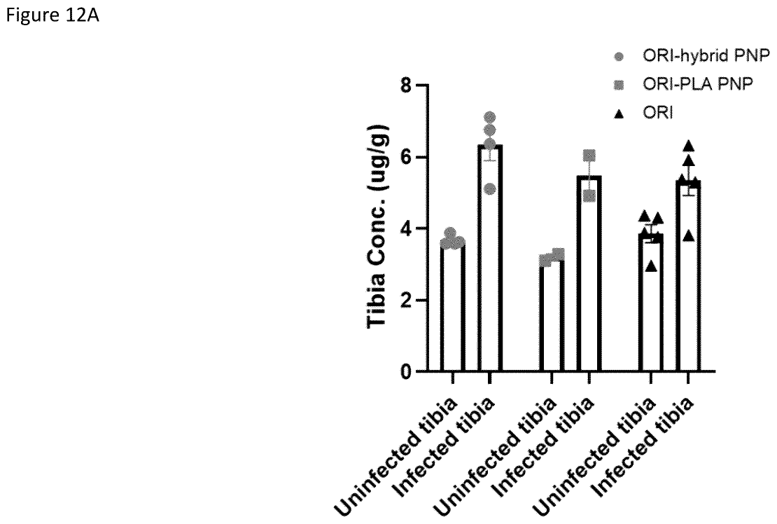

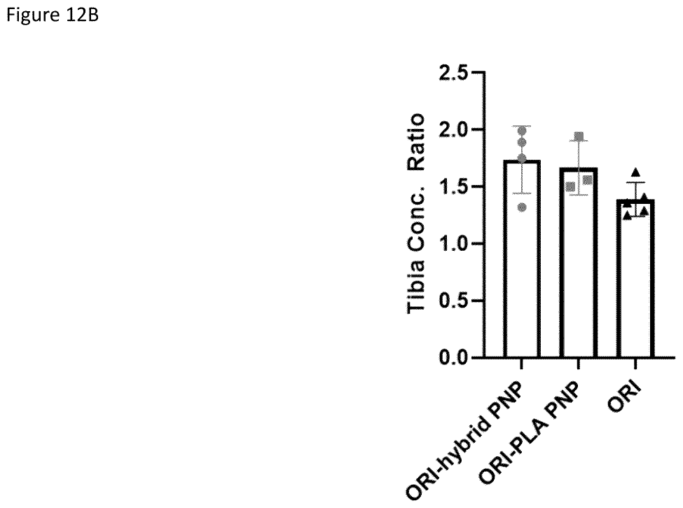

FIG. 12A shows the mean concentrations of oritavancin in the uninfected and infected tibias of the rats in each treatment group. FIG. 12B shows the ratio of the mean oritavancin concentration in the infected tabia to the mean oritavancin concentration in the uninfected tabia of each treatment group. These data indicate preferential accumulation of oritavancin in the infected tibia compared to the uninfected tibia 24 h post-intravenous injection of ORI-hybrid PNP, ORI-PLA PNP, or ORI. However, the ratio of the mean oritavancin concentration in the infected tibia to that in the uninfected tibia was higher than in rats injected with ORI-hybrid PNP or ORI-PLA PNP than in rats injected with ORI (free oritavancin), indicating better targeted delivery of oritavancin to the infected tibia via intravenous administration of ORI-hybrid PNP or ORI-PLA PNP to the osteomyelitis rats.

### Example 5—Development and Evaluation of Targeting Moiety-Conjugated PNPs

This example describes further development of PNP formulations with a targeting moiety for enhanced targeted delivery of an antibiotic drug to the site of MRSA biofilm infection. By in vitro screening for efficient binding to biofilm matrix, an anti-polysaccharide intercellular adhesin (PIA) antibody purchased from Creative Biolabs (Shirley, NY, USA; Clone #SAR279356; Cat #TAB-799CL) was selected as the targeting moiety attached to the PNPs, specifically to PLA-PEG.

PLA-PEG is a biodegradable PEG-based amphiphilic block copolymer capable of forming nanoparticles by self-assembly in water. The targeting moiety, i.e., the anti-PIA antibody, was attached to PLA-PEG via a reactive group at the end of its PEG block after nanoparticle assembly via three methods: maleimide chemistry (using maleimide-terminated PLA-PEG), click chemistry and avidin-biotin method. Table 8 is a summary comparing the characteristics of the anti-PIA antibody/PLA-PEG conjugates prepared by the three methods.

TABLE 8
Characteristics of anti-PIA antibody-conjugated PLA-PEG nanoparticles prepared
by maleimide chemistry, click chemistry, or avidin/biotin method

| | | | | Antibody | |
|---|---|---|---|---|---|
| | | | Purification | (Ab):Nanoparticle | In vitro |
| | Conjugation | | after | (NP) Ratio | biofilm |
| | yield | Reaction time | conjugation | (NP about 150 nm) | binding % |
| Maleimide | 5-10% | 2-3 hrs | Required | About 30 | 3% of total |
| chemistry | | | (drug loss | | NP |
| | | | observed) | | |
| Click | 50% | 2-3 hrs | Required | 50-100 | 26% of total |
| chemistry | | | (drug loss | | NP |
| | | | observed) | | |
| Avidin/biotin | About 100% | Instantly | Not required | >100 | 45% of total |
| method | | | | | NP |

The anti-PIA antibody density on the PLA-PEG nanoparticles, expressed as the antibody (Ab): nanoparticle (NP) ratio in Table 8, was quantified by three methods: amino acid analysis with HPLC, a BCA assay, and an HRP ELISA method. With the method of amino acid analysis with HPLC, anti-PIA antibody-coated nanoparticles were treated with 6 N HCl under high temperature and pressure conditions for 16 h to hydrolyze the anti-PIA antibody into amino acid monomers. Afterwards a borate buffer and AQC derivatization reagent were added to the hydrolysis products, and the reaction mixture was subjected to shaking at 55° C. for 10 min. Thereafter the sample was analyzed by HPLC. Glycine could be separated from other amino acids for quantification and the concentration of protein was calculated from the glycine peak. Based on protein concentration and the number density of nanoparticles measured by a Malvern NanoSight, the surface density of the anti-PIA antibody on the PLA-PEG nanoparticles was determined.

With the BCA assay method, a BCA reagent was applied to the filtrate of anti-PIA antibody-coated nanoparticles to detect free anti-PIA antibody. The principle of this method is that proteins, including antibodies, can reduce Cu⁺² to Cu⁺¹ in an alkaline solution, resulting in a purple color formation by bicinchoninic acid. The reduction of copper is mainly caused by four amino acid residues, including cysteine or cystine, tyrosine, and tryptophan that are present in protein molecules. The anti-PIA antibody-coated nanoparticles were filtrated by Amicon Filter (1000 KDa cut-off). The filtrate was collected and analyzed by standard BCA assay. Non-antibody modified nanoparticles were analyzed in the same way as background control. The stock anti-PIA antibody was diluted into standard series and analyzed in the same way as standard control. The concentration and the number density of the free anti-PIA antibody in nanoparticle solution was calculated via the anti-PIA antibody standard. The number density of conjugated anti-PIA antibody was calculated by subtracting the free anti-PIA antibody from the loaded anti-PIA antibody. The number density of nanoparticles was measured by NanoSight, based on which the surface density of the anti-PIA antibody was determined.

With the HRP ELISA method, an HRP-conjugated secondary antibody was applied to detect the anti-PIA antibody on the nanoparticle surface. Specifically, anti-PIA antibody-coated nanoparticles were incubated with an excess amount of a monoclonal HRP secondary antibody at room temperature for 15 min, followed by two washes with PBST (PBS with 0.05% Tween20). Washed nanoparticles were collected by centrifugation, and treated with a Quata Blu solution for 15 min, generating chemiluminescence in the presence of HRP. Thereafter a Quata Blu stop solution was added to stop the reaction. The fluorescence signal was read at Ex/Em 325/420 nm. An HRP antibody standard solution was prepared and analyzed in the same way. The anti-PIA concentration and the number density were calculated. The number density of nanoparticles was measured by NanoSight, based on which the surface density of the anti-PIA antibody was determined.

The in vitro biofilm binding data shown in Table 8 were obtained by using the method as follows. Anti-PIA antibody-conjugated PLA-PEG nanoparticles admixed with PLA-Cy5 were diluted to around 0.1 mg/ml, and 200 ul of the diluted nanoparticles were transferred into a column wells of a 96-well fixed biofilm plate. PLA-PEG nanoparticles without the anti-PIA antibody but likewise admixed with PLA-Cy5 were processed in parallel as a comparison. Additionally, 200 ul of PBS was transferred into a column wells as a background control. The wells were covered with an adhesive plate cover and the contents of the wells were incubated at room temperature for 10 min. Thereafter fluorescence at Ex/Em 650/680 nm was read as “after incubation” fluorescence intensity. The contents of each well were decanted and each well was washed three times with 200 uL of PBST (PBS with 0.05% Tween20), and fluorescence at Ex/Em 650/680 nm was read as “after washing” fluorescence intensity. Data were calculated using the following equation:

In vitro biofilm binding %=fluorescent intensity of “after washing”/fluorescent intensity of “after incubation”

To evaluate PNP formulations with or without the anti-PIA antibody targeting moiety in vitro and in vivo, the following four RV40-loaded PNP (RV40-PNP) formulations were made: (1) Hybrid-PNP-antiPIA comprising PLA(10K)-PEG(2K) (A), PLA(10K)-PEG(5K)-biotin (A′) and pGlu(0.6C₁₀)-PEG (C), with the anti-PIA antibody attached to A′ by the biotin-avidin method; (2) Hybrid-PNP comprising PLA(10K)-PEG(2K) (A) and pGlu(0.6C₁₀)-PEG (C); (3) PLA-PNP-antiPIA comprising PLA(10K)-PEG(2K) (A) and PLA(10K)-PEG(5K)-biotin (A′), with the anti-PIA antibody attached to A′ by the biotin-avidin method; and (4) PLA-PNP comprising PLA(10K)-PEG(2K) (A). Additionally, PLA-Cy5 and PLA-Cy7.5 were added in each of the PNP formulations to facilitate the quantification by fluorescence and in vivo whole-body imaging, respectively. PLA-Cy5 and PLA-Cy7.5 were derivatives of PLA where the fluorescent label Cy5 and Cy7.5 were grafted at the branch of PLA, respectively. PLA-Cy5 was prepared as described in Example 2, and PLA-Cy7.5 was prepared using the same process with Cy7.5 in place of Cy5. The compositions and characteristics of the four RV40-PNP formulations are summarized in Table 9.

The biological targeting performance of the RV40-PNP formulations was evaluated by in vitro biofilm binding, as well as by in vivo whole-body imaging of the rat osteomyelitis model. The in vitro biofilm binding assay was carried out in a manner similar to that for anti-PIA antibody conjugated or non-anti-PIA antibody conjugated PLA-PEG nanoparticles described above, with each formulation sample containing additional components, e.g., PLA-Cy7.5, RV40, and/or pGlu(0.6C₁₀)-PEG, as indicated in Table 9.

For the in vivo whole-body imaging study, rats with bacterial osteomyelitis of the tibia were generated via intraosseous injection of MRSA ATCC 43300 as described in Example 4. Three weeks (21 days) after the initiation of the bacterial infection, (on Day 22) the rats were administered one of the four RV40-loaded PNP (RV40-PNP) formulations indicated in Table 9 via intravenous injection in the lateral tail vein at 5.2 mg/kg body weight. The rats were imaged immediately post-intravenous injection, or 3 h, 6 h, and 24 h post-intravenous injection of the formulations using the FX-Pro in vivo imaging system (bright field and fluorescent imaging at Ex750 nm/Em830 nm for Cy7.5). The rats were sacrificed at 24 h post-intravenous injection and both tibias were collected for ex-vivo imaging.

As shown in Table 9, after in vitro incubation with a biofilm plate, Hybrid-PNP-antiPIA showed 1-3 fold higher binding compared to Hybrid-PNP, indicating that the antiPIA targeting moiety enhanced the binding of PNPs to the biofilm. Additionally, both PLA-PNP-antiPIA and PLA-PNP had lower binding compared to Hybrid-PNP-antiPIA and Hybrid-PNP.

TABLE 9
Compositions and characteristics of the RV40-PNP formulations
with or without the anti-PIA antibody as the targeting moiety

| | PLA(10K)- | PLA(10K)- | | | | Avidin | Concen- | | |
|---|---|---|---|---|---|---|---|---|---|
| | PEG(2K) | PEG(5K)- | pGlu(0.6C₁₀)- | PLA- | PLA- | antiPIA/ | tration | | Biofilm |
| | (A) | biotin (A′) | PEG (C) | Cy7.5 | Cy5 | PNP ratio | (mg/ml) | Size | binding |
| Hybrid- | 44% | 1% | 45% | 8% | 2% | 50 | 2.6 | 95 | 17.6% |
| PNP- | | | | | | | | | |
| antiPIA | | | | | | | | | |
| Hybrid- | 45% | N/A | 45% | 8% | 2% | N/A | 2.6 | 108 | 7.5% |
| PNP | | | | | | | | | |
| PLA-PNP- | 89% | 1% | N/A | 8% | 2% | 50 | 2.6 | 131 | 6.5% |
| antiPIA | | | | | | | | | |
| PLA-PNP | 90% | N/A | N/A | 8% | 2% | N/A | 2.6 | 93 | 3.0% |

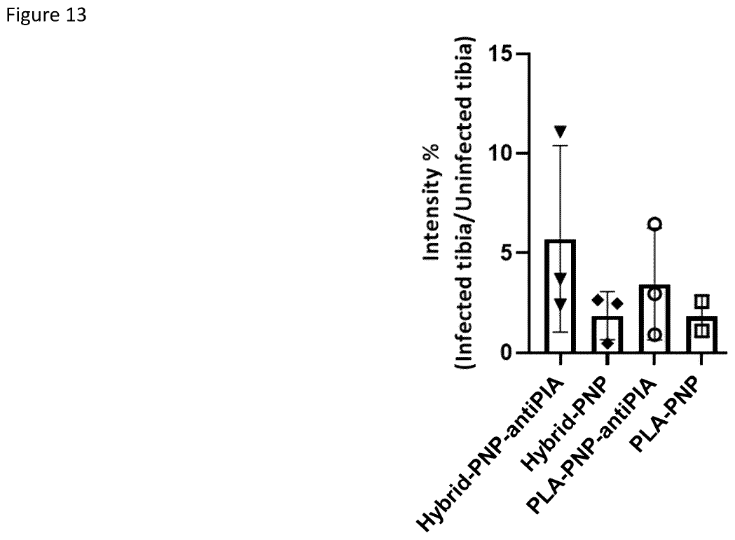

Further, as shown in FIG. 13, the in vivo whole-body imaging demonstrated that Hybrid PNP-antiPIA formulation had the greatest biofilm targeting specificity; among the four formulations, it exhibited the highest ratio of the fluorescence intensity in the infected tibia to that in the uninfected tibia, i.e., the best targeted delivery to the infected tibia in the rat osteomyelitis model. Similar observations were also obtained with the analogous formulations where the anti-PIA antibody was attached to PLA-PEG via the different methods of maleimide chemistry and azide click chemistry. Taken together, both in vitro and in vivo data indicate that the Hybrid-PNP-antiPIA formulations advantageously target biofilm infections with superior specificity.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A block, alternate, or random copolymer comprising x units of the formula 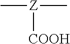
and y units of the formula 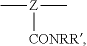
wherein Z is an organic moiety, R and R′ are each independently selected from the group consisting of hydrogen and C₁-C₁₈ alkyl, each of x and y is an integer of 1 or greater, the sum of x and y is an integer from about 40 to about 714, and y is from about 10% to about 90% of the sum of x and y.

2. The copolymer of claim 1, which is a random copolymer.

3. The copolymer of claim 1 or 2, which does not include a polyethylene glycol (PEG) moiety.

4. The copolymer of any one of claims 1-3, which is of Formula (I):
P—R² (I),
wherein R² is hydrogen or C₁-C₆ alkyl, P is a random copolymer moiety comprising x units of the formula 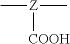
and y units of the formula

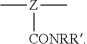

5. The copolymer of claim 1, which is of Formula (II):
PEG-L-P′ (II),
wherein L is a bond or a linker covalently connecting PEG and P′, PEG is a polyethylene glycol moiety with a molecular weight of from about 500 g/mol to about 20,000 g/mol, and P′ comprises a block, alternate, or random copolymer comprising x units of the formula 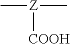
and y units of the formula

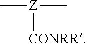

6. The copolymer of claim 5, wherein P′ comprises a random copolymer comprising x units of the formula 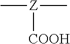
and y units of the formula

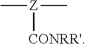

7. The copolymer of claim 5 or 6, wherein the PEG is a polyethylene glycol moiety with a molecular weight of about 2000 g/mol.

8. The copolymer of any one of claims 5-7, wherein L is a linker represented by —(CH₂)ₙ—NH—, and n is an integer from 1 to 6.

9. The copolymer of claim 8, wherein L is —CH₂—CH₂—NH—.

10. The copolymer of any one of claims 1-9, wherein one of R and R′ is linear C₁-C₁₈ alkyl and the other hydrogen.

11. The copolymer of any one of claims 1-10, wherein one of R and R′ is linear C₁₀ alkyl and the other hydrogen.

12. The copolymer of any one of claims 1-11, wherein.

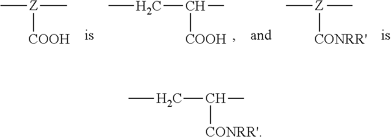

13. The copolymer of any one of claims 1-11, wherein

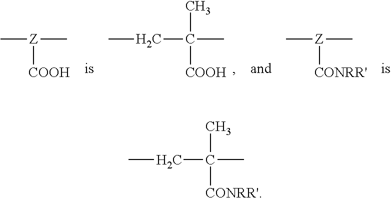

14. The copolymer of any one of claims 1-11, wherein

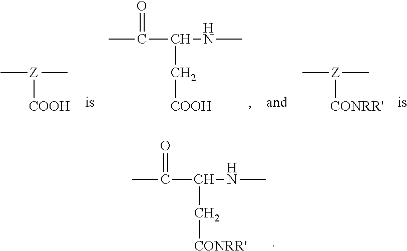

15. The copolymer of any one of claims 1-11, wherein

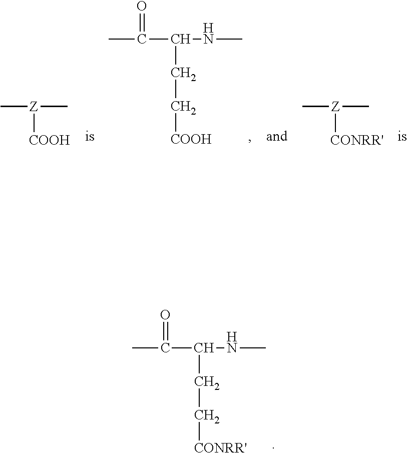

16. The copolymer of claim 4, wherein P is a random copolymer moiety comprising x units of the formula 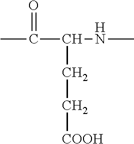
and y units of the formula 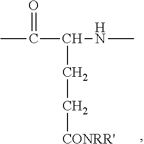
and one of R and R′ is linear C₁₀ alkyl and the other hydrogen.

17. The copolymer of claim 16, wherein R² is hydrogen.

18. The copolymer of claim 17, wherein the sum of x and y is about 155, and y is about 30% of the sum of x and y.

19. The copolymer of claim 5 or 6, wherein L is —CH₂—CH₂—NH—, P′ is P″—R², and the copolymer is of Formula (IIa): 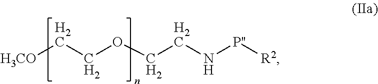
wherein n is an integer from about 22 to about 227, R² is hydrogen or C₁-C₆ alkyl, P″ is a random copolymer moiety comprising x units of the formula 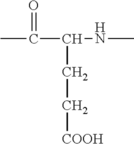
and y units of the formula 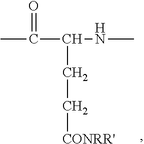
one of R and R′ is linear C₁₀ alkyl and the other hydrogen, and the ratio of the sum of x and y to n is from about 0.7 to about 7.

20. The copolymer of claim 19, wherein R² is hydrogen.

21. The copolymer of claim 20, wherein n is about 45, the sum of x and y is about 155, and y is about 36% of the sum of x and y.

22. A method of preparing the copolymer of claim 17 or 18, the method comprising reacting a poly (L-glutamic acid) of Formula (III): 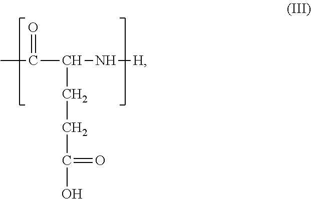
with a compound of Formula (IV): 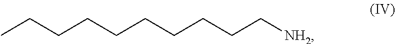
in the presence of a coupling agent selected from the group consisting of N,N′-diisopropylcarbodiimide (DIC), dicyclohexylcarbodiimide (DCC), 1-ethyl-3-(3-dimethylaminopropyl)carbodiimide (EDC), (1-[bis(dimethylamino)methylene]-1H-1,2,3-triazolo[4,5-b]pyridinium 3-oxide hexafluorophosphate (HATU), 3-[bis(dimethylamino)methyliumyl]-3H-benzotriazol-1-oxide hexafluorophosphate (HBTU), O-(1H-6-chlorobenzotriazole-1-yl)-1,1,3,3-tetramethyluronium hexafluorophosphate (HCTU), benzotriazole-1-yl-oxy-tris-(dimethylamino)-phosphonium hexafluorophosphate (BOP), 1-[(1-(cyano-2-ethoxy-2-oxoethylideneaminooxy)dimethylaminomorpholino)] uronium hexafluorophosphate (COMU), 3-(diethoxy-phosphoryloxy)-1,2,3-benzo[d]triazin-4(3H)-one (DEPBT), (7-azabenzotriazol-1-yloxy)trispyrrolidinophosphonium hexafluorophosphate (PyAOP), benzotriazole-1-yl-oxy-tris-pyrrolidino-phosphonium hexafluorophosphate (PyBOP), 1-cyano-2-ethoxy-2-oxoethylideneaminooxy-tris-pyrrolidino-phosphonium hexafluorophosphate (PyOxim), N,N,N′,N′-tetramethyl-O-(N-succinimidyl)uronium tetrafluoroborate, O-[N-succinimidyl)-1,1,3,3-tetramethyluronium tetrafluoroborate (TSTU), and 6-chloro-benzotriazole-1-yloxy-tris-pyrrolidinophosphonium hexafluorophosphate (PyClock),
wherein m is equal to the sum of x and y, and the ratio of the product of the number of moles of the poly (L-glutamic acid) of Formula (III) multiplied by m to the number of moles of the compound of Formula (IV) is from about 1:0.1 to about 1:0.9.

23. The method of claim 22, wherein the coupling agent is DIC.

24. The method of claim 22 or 23, wherein the ratio of the product of the number of moles of the poly (L-glutamic acid) of Formula (III) multiplied by m to the number of moles of the compound of Formula (IV) is about 1:0.6.

25. A method of preparing the copolymer of claim 20 or 21, the method comprising reacting a methoxy-poly (ethylene glycol)-block-poly (L-glutamic acid) of Formula (V): 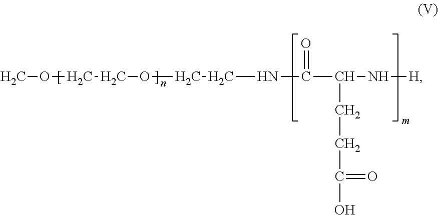
with a compound of Formula (IV): 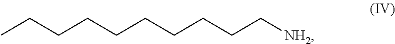
in the presence of a coupling agent selected from the group consisting of N,N′-diisopropylcarbodiimide (DIC), dicyclohexylcarbodiimide (DCC), 1-ethyl-3-(3-dimethylaminopropyl)carbodiimide (EDC), (1-[bis (dimethylamino) methylene]-1H-1,2,3-triazolo[4,5-b]pyridinium 3-oxide hexafluorophosphate (HATU), 3-[bis(dimethylamino)methyliumyl]-3H-benzotriazol-1-oxide hexafluorophosphate (HBTU), O-(1H-6-chlorobenzotriazole-1-yl)-1,1,3,3-tetramethyluronium hexafluorophosphate (HCTU), benzotriazole-1-yl-oxy-tris-(dimethylamino)-phosphonium hexafluorophosphate (BOP), 1-[(1-(cyano-2-ethoxy-2-oxoethylideneaminooxy) dimethylaminomorpholino)] uronium hexafluorophosphate (COMU), 3-(diethoxy-phosphoryloxy)-1,2,3-benzo[d]triazin-4(3H)-one (DEPBT), (7-azabenzotriazol-1-yloxy)trispyrrolidinophosphonium hexafluorophosphate (PyAOP), benzotriazole-1-yl-oxy-tris-pyrrolidino-phosphonium hexafluorophosphate (PyBOP), 1-cyano-2-ethoxy-2-oxoethylideneaminooxy-tris-pyrrolidino-phosphonium hexafluorophosphate (PyOxim), N,N,N′,N′-tetramethyl-O-(N-succinimidyl)uronium tetrafluoroborate, O-[N-succinimidyl)-1,1,3,3-tetramethyluronium tetrafluoroborate (TSTU), and 6-chloro-benzotriazole-1-yloxy-tris-pyrrolidinophosphonium hexafluorophosphate (PyClock),
wherein m is equal to the sum of x and y, and the ratio of the product of the number of moles of the compound of Formula (V) multiplied by m to the number of moles of the compound of Formula (IV) is from about 1:0.1 to about 1:0.9.

26. The method of claim 25, wherein the coupling agent is DIC.

27. The method of claim 25 or 26, wherein the ratio of the product of the number of moles of the compound of Formula (V) multiplied by m to the number of moles of the compound of Formula (IV) is about 1:0.6.

28. A pharmaceutical composition comprising nanoparticles comprising a glycopeptide antibiotic complexed with one or more copolymers of any one of claims 1-21 and a charge neutral polymer selected from the group consisting of polylactic acid, polylactic acid-PEG, poly (lactic-co-glycolic acid), a polyester-PEG, and a combination thereof, wherein the glycopeptide antibiotic is positively charged at a pH of from about 6 to about 8.

29. The pharmaceutical composition of claim 28, wherein the polyester-PEG is a polylactone family polymer-PEG selected from the group consisting of polylactide-PEG, polyglycolide-PEG, poly (lactic-co-glycolic acid)-PEG, polypropiolactone-PEG, polybutyrolactone-PEG, polyvalerolactone-PEG, polycaprolactone-PEG, polyheptalactone-PEG, polyoctalactone-PEG, polynonalactone-PEG, polydecalactine-PEG, polyundecalactine-PEG, and polydodecalactine-PEG.

30. The pharmaceutical composition of claim 28, wherein the one or more copolymers does not include a PEG moiety, and the charge neutral polymer is polylactic acid-PEG.

31. The pharmaceutical composition of claim 30, wherein the one or more copolymers which does not include a PEG moiety is a copolymer of Formula (I).

32. The pharmaceutical composition of claim 31, wherein the copolymer of Formula (I) is a copolymer of any one of claims 16-18.

33. The pharmaceutical composition of claim 28, wherein the one or more copolymers is a copolymer of Formula (II) or (IIa), and the charge neutral polymer is polylactic acid-PEG.

34. The pharmaceutical composition of claim 33, wherein the one or more copolymers is a copolymer of any one of claims 19-21.

35. The pharmaceutical composition of claim 28, wherein the glycopeptide antibiotic is complexed with at least two copolymers and the charge neutral polymer is polylactic acid-PEG, wherein one of the at least two copolymers does not include a PEG moiety and the other of the at least two copolymers is a copolymer of Formula (II) or (IIa).

36. The pharmaceutical composition of claim 35, wherein the one of the at least two copolymers which does not include a PEG moiety is a copolymer of Formula (I).

37. The pharmaceutical composition of claim 36, wherein the copolymer of Formula (I) is a copolymer of any one of claims 16-18.

38. The pharmaceutical composition of any one of claims 35-37, wherein the other of the at least two copolymers is a copolymer of any one of claims 19-21.

39. The pharmaceutical composition of claim 28, wherein the one or more copolymers does not include a PEG moiety, and the charge neutral polymer is a combination of polylactic acid-PEG and polylactic acid.

40. The pharmaceutical composition of claim 39, wherein the one or more copolymers which does not include a PEG moiety is a copolymer of Formula (I).

41. The pharmaceutical composition of claim 40, wherein the copolymer of Formula (I) is a copolymer of any one of claims 16-18.

42. The pharmaceutical composition of claim 28, wherein the one or more copolymers is a copolymer of Formula (II) or (IIa), and the charge neutral polymer is a combination of polylactic acid-PEG and polylactic acid.

43. The pharmaceutical composition of claim 42, wherein the one or more copolymers is a copolymer of any one of claims 19-21.

44. The pharmaceutical composition of claim 28, wherein the glycopeptide antibiotic is complexed with at least two copolymers and the charge neutral polymer is a combination of polylactic acid-PEG and polylactic acid, wherein one of the at least two copolymers does not include a PEG moiety and the other of the at least two copolymers is a copolymer of Formula (II) or (IIa).

45. The pharmaceutical composition of claim 44, wherein the one of the at least two copolymers which does not include a PEG moiety is a copolymer of Formula (I).

46. The pharmaceutical composition of claim 45, wherein the copolymer of Formula (I) is a copolymer of any one of claims 16-18.

47. The pharmaceutical composition of any one of claims 44-46, wherein the other of the at least two copolymers is a copolymer of any one of claims 19-21.

48. The pharmaceutical composition of claim 28, wherein the one or more copolymers is a copolymer of Formula (II) or (IIa), and the charge neutral polymer is polylactic acid.

49. The pharmaceutical composition of claim 48, wherein the one or more copolymers is a copolymer of any one of claims 19-21.

50. The pharmaceutical composition of any one of claims 28-49, wherein the nanoparticles have an average diameter of from about 60 nm to about 260 nm.

51. The pharmaceutical composition of any one of claims 28-50, wherein the glycopeptide antibiotic is selected from the group consisting of A477, A35512, A40926, A41030, A42867, A47934, A80407, A82846, A83850, A84575, AB-65, actaplanin, actinoidin, ardacin, avoparcin, azureomycin, chloroeremomycin, chloroorienticin, chloropolysporin, dalbavancin, decaplanin, N-demethylvancomycin, eremomycin, galacardin, helvecardin, izupeptin, kibdelin, LL-AM374, mannopeptin, MM45289, MM47761, MM47766, MM55266, MM55270, OA-7653, orienticin, oritavancin, parvodicin, ristocetin, ristomycin, synmonicin, teicoplanin, telavancin, UK-68597, UK-69542, UK-72051, vancomycin, a pharmaceutically acceptable salt thereof, and a combination of the foregoing.

52. The pharmaceutical composition of claim 51, wherein the glycopeptide antibiotic is selected from the group consisting of oritavancin, telavancin. and vancomycin.

53. The pharmaceutical composition of claim 52, wherein the glycopeptide antibiotic is vancomycin.

54. The pharmaceutical composition of claim 52, wherein the glycopeptide antibiotic is oritavancin.

55. The pharmaceutical composition of any one of claims 28-50, wherein the glycopeptide antibiotic is RV62, RV94, RV40, or any one of the compounds in Table 1.

56. The pharmaceutical composition of claim 55, wherein the glycopeptide antibiotic is RV94.

57. The pharmaceutical composition of claim 55, wherein the glycopeptide antibiotic is RV40.

58. The pharmaceutical composition of any one of claims 28-57, wherein a targeting moiety is attached to the nanoparticles for targeting the nanoparticles to a bacterial biofilm or the surface of a bacterium.

59. The pharmaceutical composition of claim 58, wherein the targeting moiety is an antibody or a peptide capable of binding to a bacterial biofilm or the surface of a bacterium.

60. The pharmaceutical composition of claim 59, wherein the targeting moiety is an anti-polysaccharide intercellular adhesin (PIA) antibody.

61. The pharmaceutical composition of claim 59, wherein the targeting moiety is an anti-wall teichoic acid (WTA) antibody.

62. The pharmaceutical composition of claim 59, wherein the targeting moiety is a peptide capable of binding to poly-N-acetyl glucosamine (PNAG) or deacetylated PNAG (dPNAG).

63. The pharmaceutical composition of any one of claims 58-62, wherein the targeting moiety is attached to the charge neutral polymer of the nanoparticles.

64. The pharmaceutical composition of claim 63, wherein the charge neutral polymer is polylactic acid-PEG, a polyester-PEG, or a combination thereof, and the targeting moiety is attached to the PEG moiety of the charge neutral polymer.

65. The pharmaceutical composition of claim 64, wherein the charge neutral polymer is polylactic acid-PEG.

66. The pharmaceutical composition of any one of claims 58-65, wherein the targeting moiety is attached to the nanoparticles covalently.

67. The pharmaceutical composition of any one of claims 58-65, wherein the targeting moiety is attached to the nanoparticles via non-covalent interaction.

68. The pharmaceutical composition of claim 67, wherein the non-covalent interaction is an interaction between biotin and avidin, or between biotin and streptavidin.

69. A method of preparing the pharmaceutical composition of any one of claims 28-68, the method comprising:
(a) dissolving (i) the charge neutral polymer, (ii) the glycopeptide antibiotic, and (iii) the one or more copolymers of any one of claims 1-21 in an organic solvent to form an organic solution, and
(b) injecting the organic solution into water to form the nanoparticles comprising the charge neutral polymer and the glycopeptide antibiotic complexed with the one or more copolymers.

70. The method of claim 69, wherein the organic solvent comprises one selected from the group consisting of dimethyl sulfoxide (DMSO), dimethylformamide (DMF), acetonitrile, acetone, benzyl alcohol, dichloromethane (DCM), ethylacetate, tetrahydrofuran (THF), chloroform, and a combination thereof.

71. The method of claim 69 or 70, wherein the organic solvent comprises one selected from the group consisting of DMSO, acetonitrile, and a combination of DMSO and acetonitrile.

72. The method of any one of claims 69-71, further comprising attaching a targeting moiety to the nanoparticles.

73. The method of claim 72, wherein the charge neutral polymer of the nanoparticles is polylactic acid-PEG, a polyester-PEG, or a combination thereof, and the attaching the targeting moiety to the nanoparticles comprises adding to the end of the PEG moiety of the neutral polymer a functional group configured for attaching the targeting moiety to the charge neutral polymer.

74. A method for treating a bacterial infection in a patient in need thereof, comprising administering an effective amount of the pharmaceutical composition of any one of claims 28-68 to the patient.

75. The method of claim 74, wherein an effective amount of the pharmaceutical composition of any one of claims 55-68 is administered to the patient.

76. The method of claim 74 or 75, wherein the bacterial infection is a pulmonary bacterial infection.

77. The method of claim 76, wherein the administering comprises administering to the lungs of the patient.

78. The method of claim 76 or 77, wherein the administering is carried out via a nebulizer.

79. The method of claim 76 or 77, wherein the administering is carried out via a metered dose inhaler.

80. The method of claim 76 or 77, wherein the administering is carried out via a dry powder inhaler.

81. The method of any one of claims 74-76, wherein the administering comprises intravenous administration.

82. The method of any one of claims 74-76, wherein the administering comprises subcutaneous administration.

83. The method of any one of claims 74-82, wherein the administering is carried out once daily.

84. The method of any one of claims 74-82, wherein the administering is carried out twice daily.

85. The method of any one of claims 74-82, wherein the administering is carried out three or more times daily.

86. The method of any one of claims 74-85, wherein the bacterial infection is a Gram-positive bacterial infection.

87. The method of claim 86, wherein the Gram-positive bacterial infection is a Gram-positive cocci infection.

88. The method of claim 87, wherein the Gram-positive cocci infection is a Streptococccus, Enterococcus, Staphylococcus infection, or a combination thereof.

89. The method of claim 88, wherein the Gram-positive cocci infection is a Streptococcus infection.

90. The method of claim 89, wherein the Streptococcus infection is an S. agalactiae, S. anginosus, S. bovis, S. dysgalactiae, S. mitis, S. mutans, S. pneumoniae, S. pyogenes, S. sanguinis, or S. suis infection.

91. The method of claim 90, wherein the Streptococcus infection is an S. mutansinfection.

92. The method of claim 90, wherein the Streptococcus infection is an S. pneumoniae infection.

93. The method of claim 90, wherein the Streptococcus infection is an S. dysgalactiae infection.

94. The method of claim 90, wherein the Streptococcus infection is an S. pyogenes infection.

95. The method of claim 88, wherein the Gram-positive cocci infection is an Enterococcus infection.

96. The method of claim 95, wherein the Enterococcus infection is a vancomycin resistant Enterococcus infection (VRE).

97. The method of claim 95, wherein the Enterococcus infection is a vancomycin sensitive Enterococcus infection (VSE).

98. The method of claim 95, wherein the Enterococcus infection is an Enterococcus faecalis (E. faecalis) infection.

99. The method of claim 98, wherein the E. faecalis infection is a vancomycin-sensitive E. faecalis infection.

100. The method of claim 98, wherein the E. faecalis infection is a vancomycin-resistant E. faecalis infection.

101. The method of claim 98, wherein the E. faecalis infection is an ampicillin-resistant E. faecalis infection.

102. The method of claim 95, wherein the Enterococcus infection is an Enterococcus faecium (E. faecium) infection.

103. The method of claim 102, wherein the E. faecium infection is a vancomycin-resistant E. faecium infection.

104. The method of claim 102, wherein the E. faecium infection is a vancomycin-sensitive E. faecium infection.

105. The method of claim 102, wherein the E. faecium infection is an ampicillin-resistant E. faecium infection.

106. The method of claim 88, wherein the Gram-positive cocci infection is a Staphylococcus infection.

107. The method of claim 106, wherein the Staphylococcus infection is a Staphylococcus aureus (S. aureus) infection.

108. The method of claim 107, wherein the S. aureus infection is a methicillin-resistant S. aureus (MRSA) infection.

109. The method of claim 107, wherein the S. aureus infection is a methicillin-sensitive S. aureus (MSSA) infection.

110. The method of claim 107, wherein the S. aureus infection is a vancomycin-intermediate S. aureus (VISA) infection.

111. The method of claim 107, wherein the S. aureus infection is a vancomycin-resistant S. aureus (VRSA) infection.

112. The method of claim 106, wherein the Staphylococcus infection is a Staphylococcus haemolyticus (S. haemolyticus) infection.

113. The method of claim 106, wherein the Staphylococcus infection is a Staphylococcus epidermis (S. epidermis) infection.

114. The method of any one of claims 106, 112, and 113, wherein the Staphylococcus infection is penicillin resistant.

115. The method of any one of claims 106, 112, and 113, wherein the Staphylococcus infection is methicillin resistant.

116. The method of any one of claims 106, 112, and 113, wherein the Staphylococcus infection is vancomycin resistant.

117. The method of any one of claims 74-86, wherein the bacterial infection is a Bacillus anthracis (B. anthracis) infection.

118. The method of any one of claims 74-85, wherein the bacterial infection is a Francisella tularensis (F. tularensis) infection.

119. The method of any one of claims 74-85, wherein the bacterial infection is a Burkholderia infection.

120. The method of claim 119, wherein the Burkholderia infection is a Burkholderia pseudomallei (B. pseudomallei), B. dolosa, B. fungorum, B. gladioli, B. multivorans, B. vietnamiensis, B. ambifaria, B. andropogonis, B. anthina, B. brasilensis, B. calcdonica, B. caribensis, or B. caryophylli infection, or a combination thereof.

121. The method of any one of claims 74-85, wherein the bacterial infection is a Yersinia pestis (Y. pestis) infection.

122. The method of any one of claims 74-86, wherein the bacterial infection is a Clostridium difficile (C. difficile) infection.

123. The method of any one of claims 74-122, wherein the bacterial infection is a planktonic bacterial infection.

124. The method of any one of claims 74-123, wherein the patient is a cystic fibrosis patient.

125. The method of any one of claim 74-123, wherein the patient is an osteomyelitis patient.

126. The method of any one of claims 74-125, wherein the bacterial infection is acquired in a healthcare setting.

127. The method of any one of claims 74-126, wherein the bacterial infection is community associated.

128. The method of any one of claims 74-127, wherein the bacterial infection comprises a bacterial biofilm infection.
